(19) United States
(12) Patent Application Publication
(10) Pub. No.: US 2013/0096088 A1
(43) Pub. Date: Apr. 18, 2013

(54) Inhibitors of Protein Tyrosine Kinase Activity

(71) Applicant: METHYLGENE INC., Montreal (CA)

(72) Inventors: Stéphane Raeppel, St. Lazare (CA); Masashi Kishida, Osaka (JP); Yohei Yuki, Osaka (JP); Stephen William Claridge, Montreal (CA); Franck Raeppel, Montreal (CA); Arkadii Vaisburg, Kirkland (CA)

(73) Assignee: METHYLGENE INC., Montreal (CA)

(21) Appl. No.: 13/629,050

(22) Filed: Sep. 27, 2012

Related U.S. Application Data
(60) Provisional application No. 61/541,344, filed on Sep. 30, 2011.

(51) Int. Cl. C07D 495/04; C07F 9/6561

(52) U.S. Cl. CPC C07D 495/04; C07F 9/6561
USPC 514/81; 544/337; 544/362; 514/253.04; 540/467; 514/301; 546/114; 514/210.18

(57) ABSTRACT

The present invention provides new compounds and methods for treating a disease responsive to inhibition of kinase activity, for example a disease responsive to inhibition of protein tyrosine kinase activity, for example a disease responsive to inhibition of protein tyrosine kinase activity of growth factor receptors, for example a disease responsive to inhibition of receptor type tyrosine kinase signaling, or for example, a disease responsive to inhibition of VEGF receptor signaling.

### FIELD OF THE INVENTION

This invention relates to compounds that inhibit protein tyrosine kinase activity. In particular the invention relates to compounds that inhibit the protein tyrosine kinase activity of growth factor receptors, resulting in the inhibition of receptor signaling, for example, the inhibition of VEGF receptor signaling and HGF receptor signaling. More particularly, the invention relates to compounds, compositions and methods for the inhibition of VEGF receptor signaling.

### SUMMARY OF THE RELATED ART

Tyrosine kinases may be classified as growth factor receptor (e.g. EGFR, PDGFR, FGFR and erbB2) or non-receptor (e.g. c-src and bcr-abl) kinases. The receptor type tyrosine kinases make up about 20 different subfamilies. The non-receptor type tyrosine kinases make up numerous subfamilies. These tyrosine kinases have diverse biological activity. Receptor tyrosine kinases are large enzymes that span the cell membrane and possess an extracellular binding domain for growth factors, a transmembrane domain, and an intracellular portion that functions as a kinase to phosphorylate a specific tyrosine residue in proteins and hence to influence cell proliferation. Aberrant or inappropriate protein kinase activity can contribute to the rise of disease states associated with such aberrant kinase activity.

Angiogenesis is an important component of certain normal physiological processes such as embryogenesis and wound healing, but aberrant angiogenesis contributes to some pathological disorders and in particular to tumor growth. VEGF-A (vascular endothelial growth factor A) is a key factor promoting neovascularization (angiogenesis) of tumors. VEGF induces endothelial cell proliferation and migration by signaling through two high affinity receptors, the fms-like tyrosine kinase receptor, Flt-1, and the kinase insert domain-containing receptor, KDR. These signaling responses are critically dependent upon receptor dimerization and activation of intrinsic receptor tyrosine kinase (RTK) activity. The binding of VEGF as a disulfide-linked homodimer stimulates receptor dimerization and activation of the RTK domain. The kinase autophosphorylates cytoplasmic receptor tyrosine residues, which then serve as binding sites for molecules involved in the propagation of a signaling cascade. Although multiple pathways are likely to be elucidated for both receptors, KDR signaling is most extensively studied, with a mitogenic response suggested to involve ERK-1 and ERK-2 mitogen-activated protein kinases.

Disruption of VEGF receptor signaling is a highly attractive therapeutic target in cancer, as angiogenesis is a prerequisite for all solid tumor growth, and that the mature endothelium remains relatively quiescent (with the exception of the female reproductive system and wound healing). A number of experimental approaches to inhibiting VEGF signaling have been examined, including use of neutralizing antibodies, receptor antagonists, soluble receptors, antisense constructs and dominant-negative strategies.

Despite the attractiveness of anti-angiogenic therapy by VEGF inhibition alone, several issues may limit this approach. VEGF expression levels can themselves be elevated by numerous diverse stimuli and perhaps most importantly, the hypoxic state of tumors resulting from VEGFr inhibition, can lead to the induction of factors that themselves promote tumor invasion and metastasis thus, potentially undermining the impact of VEGF inhibitors as cancer therapeutics.

The HGF (hepatocyte growth factor) and the HGF receptor, c-met, are implicated in the ability of tumor cells to undermine the activity of VEGF inhibition. HGF derived from either stromal fibroblasts surrounding tumor cells or expressed from the tumor itself has been suggested to play a critical role in tumor angiogenesis, invasion and metastasis. For example, invasive growth of certain cancer cells is drastically enhanced by tumor-stromal interactions involving the HGF/c-Met (HGF receptor) pathway. HGF, which was originally identified as a potent mitogen for hepatocytes is primarily secreted from stromal cells, and the secreted HGF can promote motility and invasion of various cancer cells that express c-Met in a paracrine manner. Binding of HGF to c-Met leads to receptor phosphorylation and activation of Ras/mitogen-activated protein kinase (MAPK) signaling pathway, thereby enhancing malignant behaviors of cancer cells. Moreover, stimulation of the HGF/c-met pathway itself can lead to the induction of VEGF expression, itself contributing directly to angiogenic activity.

Thus, anti-tumor anti-angiogenic strategies or approaches that target VEGF/VEGFr signaling or HGF/c-met signaling may represent improved cancer therapeutics.

Tyrosine kinases also contribute to the pathology of ophthalmic diseases, disorders and conditions, such as age-related macular degeneration (AMD) and diabetic retinopathy (DR). Blindness from such diseases has been linked to anomalies in retinal neovascularization. The formation of new blood vessels is regulated by growth factors such as VEGF and HGF that activate receptor tyrosine kinases resulting in the initiation of signaling pathways leading to plasma leakage into the macula, causing vision loss. Kinases are thus attractive targets for the treatment of eye diseases involving neovascularization.

Thus, there is a need to develop a strategy for controlling neovascularization of the eye and to develop a strategy for the treatment of ocular diseases.

Here we describe small molecules that are potent inhibitors of protein tyrosine kinase activity.

### BRIEF SUMMARY OF THE INVENTION

The present invention provides new compounds and methods for treating a disease responsive to inhibition of kinase activity, for example a disease responsive to inhibition of protein tyrosine kinase activity, for example a disease responsive to inhibition of protein tyrosine kinase activity of growth factor receptors, for example a disease responsive to inhibition of receptor type tyrosine kinase signaling, or for example, a disease responsive to inhibition of VEGF receptor signaling. In some embodiments the disease is a cell proliferative disease. In other embodiments, the disease is an ophthalmic disease. The compounds of the invention are inhibitors of kinase activity, such as protein tyrosine kinase activity, for example protein tyrosine kinase activity of growth factor receptors, or for example receptor type tyrosine kinase signaling.

In a first aspect, the invention provides compounds of Formula (I) that are useful as kinase inhibitors:

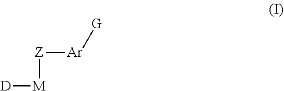

and N-oxides, hydrates, solvates, tautomers, pharmaceutically acceptable salts, prodrugs and complexes thereof, and racemic and scalemic mixtures, diastereomers and enantiomers thereof, wherein D, M, Z, Ar and G are as defined herein. Because compounds of the present invention are useful as kinase inhibitors they are, therefore, useful research tools for the study of the role of kinases in both normal and disease states. In some embodiments, the invention provides compounds that are useful as inhibitors of VEGF receptor signaling and, therefore, are useful research tools for the study of the role of VEGF in both normal and disease states.

In a second aspect, the invention provides compositions comprising a compound according to the present invention and a pharmaceutically acceptable carrier, excipient or diluent. For example, the invention provides compositions comprising a compound that is an inhibitor of VEGF receptor signaling, or a pharmaceutically acceptable salt thereof, and a pharmaceutically acceptable carrier, excipient, or diluent.

In a third aspect, the invention provides a method of inhibiting kinase activity, for example protein tyrosine kinase, for example tyrosine kinase activity of a growth factor receptor, the method comprising contacting the kinase with a compound according to the present invention, or with a composition according to the present invention. In some embodiments of this aspect, the invention provides a method of inhibiting receptor type tyrosine kinase signaling, for example inhibiting VEGF receptor signaling. Inhibition can be in a cell or a multicellular organism. If in a cell, the method according to this aspect of the invention comprises contacting the cell with a compound according to the present invention, or with a composition according to the present invention. If in a multicellular organism, the method according to this aspect of the invention comprises administering to the organism a compound according to the present invention, or a composition according to the present invention. In some embodiments the organism is a mammal, for example a primate, for example a human.

In a fourth aspect, the invention provides a method of inhibiting angiogenesis, the method comprising administering to a patient in need thereof a therapeutically effective amount of a compound according to the present invention, or a therapeutically effective amount of a composition according to the present invention. In some embodiments of this aspect, the angiogenesis to be inhibited is involved in tumor growth. In some other embodiments the angiogenesis to be inhibited is retinal angiogenesis. In some embodiments of this aspect, the patient is a mammal, for example a primate, for example a human.

In a fifth aspect, the invention provides a method of treating a disease responsive to inhibition of kinase activity, for example a disease responsive to inhibition of protein tyrosine kinase activity, for example a disease responsive to inhibition of protein tyrosine kinase activity of growth factor receptors. In some embodiments of this aspect, the invention provides a method of treating a disease responsive to inhibition of receptor type tyrosine kinase signaling, for example a disease responsive to inhibition of VEGF receptor signaling, the method comprising administering to an organism in need thereof a therapeutically effective amount of a compound according to the present invention, or a composition according to the present invention. In some embodiments of this aspect, the organism is a mammal, for example a primate, for example a human.

In a sixth aspect, the invention provides a method of treating a cell proliferative disease, the method comprising administering to a patient in need thereof a therapeutically effective amount of a compound according to the present invention, or a therapeutically effective amount of a composition according to the present invention. In some embodiments of this aspect, the cell proliferative disease is cancer. In some embodiments, the patient is a mammal, for example a primate, for example a human.

In a seventh aspect, the invention provides a method of treating an ophthalmic disease, disorder or condition, the method comprising administering to a patient in need thereof a therapeutically effective amount of a compound according to the present invention, or a therapeutically effective amount of a composition according to the present invention. In some embodiments of this aspect, the disease is caused by choroidal angiogenesis. In some embodiments of this aspect, the patient is a mammal, for example a primate, for example a human.

In an eighth aspect, the invention provides for the use of a compound according to the present invention for or in the manufacture of a medicament to inhibit kinase activity, for example to inhibit protein tyrosine kinase activity, for example to inhibit protein tyrosine kinase activity of growth factor receptors. In some embodiments of this aspect, the invention provides for the use of a compound according to the present invention for or in the manufacture of a medicament to inhibit receptor type tyrosine kinase signaling, for example to inhibit VEGF receptor signaling. In some embodiments of this aspect, the invention provides for the use of a compound according to the present invention for or in the manufacture of a medicament to treat a disease responsive to inhibition of kinase activity. In some embodiments of this aspect, the disease is responsive to inhibition of protein tyrosine kinase activity, for example inhibition of protein tyrosine kinase activity of growth factor receptors. In some embodiments of this aspect, the disease is responsive to inhibition of receptor type tyrosine kinase signaling, for example VEGF receptor signaling. In some embodiments of this aspect, the disease is a cell proliferative disease, for example cancer. In some embodiments of this aspect, the disease is an ophthalmic disease, disorder or condition. In some embodiments of this aspect, the ophthalmic disease, disorder or condition is caused by choroidal angiogenesis. In some embodiments of this aspect, the disease is age-related macular degeneration, diabetic retinopathy or retinal edema.

In a ninth aspect, the invention provides for the use of a compound according to the present invention, or a composition thereof, to inhibit kinase activity, for example to inhibit receptor type tyrosine kinase activity, for example to inhibit protein tyrosine kinase activity of growth factor receptors. In some embodiments of this aspect, the invention provides for the use of a compound according to the present invention, or a composition thereof, to inhibit receptor type tyrosine kinase signaling, for example to inhibit VEGF receptor signaling.

In a tenth aspect, the invention provides for the use of a compound according to the present invention, or a composition thereof, to treat a disease responsive to inhibition of kinase activity, for example a disease responsive to inhibition of protein tyrosine kinase activity, for example a disease responsive to inhibition or protein tyrosine kinase activity of growth factor receptors. In some embodiments of this aspect, the invention provides for the use of a compound according to the present invention, or a composition thereof, to treat a disease responsive to inhibition of receptor type tyrosine kinase signaling, for example a disease responsive to inhibition of VEGF receptor signaling. In some embodiments of this aspect, the disease is a cell proliferative disease, for example cancer. In some embodiments of this aspect, the disease is an ophthalmic disease, disorder or condition. In some embodiments of this aspect, the ophthalmic disease, disorder or condition is caused by choroidal angiogenesis.

The foregoing merely summarizes some aspects of the invention and is not intended to be limiting in nature. These aspects and other aspects and embodiments are described more fully below.

### DETAILED DESCRIPTION

The invention provides compounds, compositions and methods for inhibiting kinase activity, for example protein tyrosine kinase activity, for example receptor protein kinase activity, for example the VEGF receptor KDR. The invention also provides compounds, compositions and methods for inhibiting angiogenesis, treating a disease responsive to inhibition of kinase activity, treating cell proliferative diseases and conditions and treating ophthalmic diseases, disorders and conditions. The patent and scientific literature referred to herein reflects knowledge that is available to those with skill in the art. The issued patents, published patent applications, and references that are cited herein are hereby incorporated by reference to the same extent as if each was specifically and individually indicated to be incorporated by reference. In the case of inconsistencies, the present disclosure will prevail. For purposes of the present invention, the following abbreviations will be used (unless expressly stated otherwise)

| | |
|---|---|
| Ac | acetyl |
| AcOEt | ethyl acetate |
| AcOH | acetic acid |
| aq | aqueous |
| bd | broad doublet (NMR) |
| Bn | benzyl |
| Boc | tert-butoxycarbonyl |
| br s | broad singlet (NMR) |
| CV | column volume |
| d | doublet (NMR) |
| dd | doublet of doublets (NMR) |
| DCC | dicyclohexyl carbodiimide |
| DCM | dichloromethane |
| DEAD | diethyl diazenedicarboxylate |
| DIPEA | diisopropyl ethylamine |
| DMAP | N,N-dimethylamino pyridine |
| DMF | N,N-dimethylformamide |
| DMSO | dimethylsulfoxide |
| DMSO-d₆ | dimethylsulfoxide-d₆ |
| EDC | 1-(3-dimethylaminopropyl)- |
| | 3-ethyl-carbodiimide |
| Et | ethyl |
| EDCI | 1-(3-dimethylaminopropy1)- |
| | 3-ethyl-carbodiimide |
| Et₃N | triethylamine |
| EtOH | ethanol |
| EtOAc | ethyl acetate |
| Et₂O | diethyl ether |
| equiv | equivalent |
| g | gram (grams) |
| h | hour (hours) |
| HOBT | 1-hydroxybenzotriazole |
| m | multiplet (NMR) |
| mL | milliliter |
| μL | microliter |
| Me | methyl |
| MeOH | methanol |
| MeOH-d₄ | methanol-d₄ |
| mg | milligram (milligrams) |
| min | minute (minutes) |
| MS | mass-spectroscopy |
| m/z | mass-to-charge ratio |
| NMP | N-methyl-2-pyrrolidone |
| NMR | nuclear magnetic resonance |
| | spectroscopy |
| PEG | polyethylene glycol |
| Ph | phenyl |
| Ppm | parts per million (NMR) |
| rt | room temperature |
| s | singlet (NMR) |
| t | triplet (NMR) |
| TFA | trifluoroacetic acid |
| THF | tetrahydrofuran |

For purposes of the present invention, the following definitions will be used (unless expressly stated otherwise):

For simplicity, chemical moieties are defined and referred to throughout primarily as univalent chemical moieties (e.g., alkyl, aryl, etc.). Nevertheless, such terms are also used to convey corresponding multivalent moieties under the appropriate structural circumstances clear to those skilled in the art. For example, while an “alkyl” moiety generally refers to a monovalent radical (e.g. CH₃—CH₂—), in certain circumstances a bivalent linking moiety can be “alkyl,” in which case those skilled in the art will understand the alkyl to be a divalent radical (e.g., —CH₂—CH₂—), which is equivalent to the term “alkylene.” Similarly, in circumstances in which a divalent moiety is required and is stated as being “aryl,” those skilled in the art will understand that the term “aryl” refers to the corresponding divalent moiety, arylene. All atoms are understood to have their normal number of valences for bond formation (i.e., 4 for carbon, 3 for nitrogen, 2 for oxygen, and 2, 4, or 6 for sulfur, depending on the oxidation state of the S). On occasion a moiety may be defined, for example, as (A)ₐ-B—, wherein a is 0 or 1. In such instances, when a is 0 the moiety is B— and when a is 1 the moiety is A-B—.

For simplicity, reference to a “Cₙ-Cₘ”heterocyclyl or “Cₙ-Cₘ”heteroaryl means a heterocyclyl or heteroaryl having from “n” to “m” annular atoms, where “n” and “m” are integers. Thus, for example, a C₅-C₆heterocyclyl is a 5- or 6-membered ring having at least one heteroatom, and includes pyrrolidinyl (C₅) and piperazinyl and piperidinyl (C₆); C₆heteroaryl includes, for example, pyridyl and pyrimidyl.

The term “hydrocarbyl” refers to a straight, branched, or cyclic alkyl, alkenyl, or alkynyl, each as defined herein. A “C₀” hydrocarbyl is used to refer to a covalent bond. Thus, “C₀-C₃ hydrocarbyl” includes a covalent bond, methyl, ethyl, ethenyl, ethynyl, propyl, propenyl, propynyl, and cyclopropyl.

The term “alkyl” is intended to mean a straight chain or branched aliphatic group having from 1 to 12 carbon atoms, alternatively 1-8 carbon atoms, and alternatively 1-6 carbon atoms. In some embodiments, the alkyl group has 1-4 carbon atoms. In some embodiments, the alkyl groups have from 2 to 12 carbon atoms, alternatively 2-8 carbon atoms and alternatively 2-6 carbon atoms. In some embodiments, the alkyl group has 2-4 carbon atoms. Examples of alkyl groups include, without limitation, methyl, ethyl, propyl, isopropyl, butyl, isobutyl, sec-butyl, tert-butyl, pentyl, hexyl and the like. A “C₀” alkyl (as in “C₀-C₃alkyl”) is a covalent bond.

The term “alkenyl” is intended to mean an unsaturated straight chain or branched aliphatic group with one or more carbon-carbon double bonds, having from 2 to 12 carbon atoms, alternatively 2-8 carbon atoms, and alternatively 2-6 carbon atoms. In some embodiments, the alkenyl group has 2-4 carbon atoms. Examples alkenyl groups include, without limitation, ethenyl, propenyl, butenyl, pentenyl, and hexenyl.

The term “alkynyl” is intended to mean an unsaturated straight chain or branched aliphatic group with one or more carbon-carbon triple bonds, having from 2 to 12 carbon atoms, alternatively 2-8 carbon atoms, and alternatively 2-6 carbon atoms. In some embodiments, the alkynyl group has 2-4 carbon atoms. Examples of alkynyl groups include, without limitation, ethynyl, propynyl, butynyl, pentynyl, and hexynyl.

The terms “alkylene,” “alkenylene,” or “alkynylene” as used herein are intended to mean an alkyl, alkenyl, or alkynyl group, respectively, as defined hereinabove, that is positioned between and serves to connect two other chemical groups. Examples of alkylene groups include, without limitation, methylene, ethylene, propylene, and butylene. Examples of alkenylene groups include, without limitation, ethenylene, propenylene, and butenylene. Examples of alkynylene groups include, without limitation, ethynylene, propynylene, and butynylene.

The term “carbocycle” as employed herein is intended to mean a cycloalkyl or aryl moiety.

The term “cycloalkyl” is intended to mean a saturated, partially unsaturated or unsaturated mono-, bi-, tri- or poly-cyclic hydrocarbon group having about 3 to 15 carbons, alternatively having 3 to 12 carbons, alternatively 3 to 8 carbons, alternatively 3 to 6 carbons, and alternatively 5 or 6 carbons. In some embodiments, the cycloalkyl group is fused to an aryl, heteroaryl or heterocyclic group. Examples of cycloalkyl groups include, without limitation, cyclopenten-2-enone, cyclopenten-2-enol, cyclohex-2-enone, cyclohex-2-enol, cyclopropyl, cyclobutyl, cyclobutenyl, cyclopentyl, cyclopentenyl, cyclohexyl, cyclohexenyl, cycloheptyl, cyclooctyl, etc.

The term “heteroalkyl” is intended to mean a saturated, partially unsaturated or unsaturated, straight chain or branched aliphatic group, wherein one or more carbon atoms in the group are independently replaced by a heteroatom selected from the group consisting of O, S, and N.

The term “aryl” is intended to mean a mono-, bi-, tri- or polycyclic aromatic moiety, comprising one to three aromatic rings. In some embodiments the aryl is a C₆-C₁₄aromatic moiety, alternatively the aryl group is a C₆-C₁₀aryl group, alternatively a C₆ aryl group. Examples of aryl groups include, without limitation, phenyl, naphthyl, anthracenyl, and fluorenyl.

The terms “aralkyl” or “arylalkyl” are intended to mean a group comprising an aryl group covalently linked to an alkyl group. If an aralkyl group is described as “optionally substituted”, it is intended that either or both of the aryl and alkyl moieties may independently be optionally substituted or unsubstituted. In some embodiments, the aralkyl group is (C₁-C₆)alk(C₆-C₁₀)aryl, including, without limitation, benzyl, phenethyl, and naphthylmethyl. For simplicity, when written as “arylalkyl” this term, and terms related thereto, is intended to indicate the order of groups in a compound as “aryl-alkyl”. Similarly, “alkyl-aryl” is intended to indicate the order of the groups in a compound as “alkyl-aryl”.

The terms “heterocyclyl”, “heterocyclic” or “heterocycle” are intended to mean a group which is a mono-, bi-, or polycyclic structure having from about 3 to about 14 atoms, alternatively 3 to 8 atoms, alternatively 4 to 7 atoms, alternatively 5 or 6 atoms wherein one or more atoms, for example 1 or 2 atoms, are independently selected from the group consisting of N, O, and S, the remaining ring-constituting atoms being carbon atoms. The ring structure may be saturated, unsaturated or partially unsaturated. In some embodiments, the heterocyclic group is non-aromatic, in which case the group is also known as a heterocycloalkyl. In some embodiments the heterocyclyl is a spiro-heterocyclyl, such as 2,7-diazaspiro[4.4]nonane, 2,8-diazaspiro[5.5]undecane, 2,8-diazaspiro[4.5]decane, 2,7-diazaspiro[3.5]nonane, 2,6-diazaspiro[3.4]octane, 2-oxa-7-azaspiro[4.4]nonane, 2-oxa-8-azaspiro[5.5]undecane, 8-oxa-2-azaspiro[4.5]decane, 7-oxa-2-azaspiro[3.5]nonane, 6-oxa-2-azaspiro[3.4]octane, 1-oxa-7-azaspiro[4.4]nonane, 2-oxa-8-azaspiro[5.5]undecane, 2-oxa-8-azaspiro[4.5]decane, 2-oxa-7-azaspiro[3.5]nonane and 2-oxa-6-azaspiro[3.4]octane. In a bicyclic or polycyclic structure, one or more rings may be aromatic; for example, one ring of a bicyclic heterocycle or one or two rings of a tricyclic heterocycle may be aromatic, as in indan and 9,10-dihydro anthracene. Examples of heterocyclic groups include, without limitation, epoxy, aziridinyl, tetrahydrofuranyl, pyrrolidinyl, piperidinyl, piperazinyl, thiazolidinyl, oxazolidinyl, oxazolidinonyl, morpholino, thienyl, pyridyl, 1,2,3-triazolyl, imidazolyl, isoxazolyl, pyrazolyl, piperazino, piperidyl, piperidino, morpholinyl, homopiperazinyl, homopiperazino, thiomorpholinyl, thiomorpholino, tetrahydropyrrolyl, and azepanyl. In some embodiments, the heterocyclic group is fused to an aryl, heteroaryl, or cycloalkyl group. Examples of such fused heterocycles include, without limitation, tetrahydroquinoline and dihydrobenzofuran. Specifically excluded from the scope of this term are compounds where an annular O or S atom is adjacent to another O or S atom.

In some embodiments, the heterocyclic group is a heteroaryl group. As used herein, the term “heteroaryl” is intended to mean a mono-, bi-, tri- or polycyclic group having 3 to 24 ring atoms, alternatively 5, 6, 9, or 10 ring atoms; having for example 6, 10, or 14 pi electrons shared in a cyclic array; and having, in addition to carbon atoms, between one or more heteroatoms, preferably one to eight heteroatoms, independently selected from the group consisting of N, O, and S. For example, a heteroaryl group includes, without limitation, pyrimidinyl, pyridinyl, benzimidazolyl, thienyl, benzothiazolyl, benzofuranyl and indolinyl. Other examples of heteroaryl groups include, without limitation, thienyl, benzothienyl, furyl, benzofuryl, dibenzofuryl, pyrrolyl, imidazolyl, pyrazolyl, pyridyl, pyrazinyl, pyrimidinyl, indolyl, quinolyl, isoquinolyl, quinoxalinyl, tetrazolyl, oxazolyl, thiazolyl, and isoxazolyl.

The terms “arylene,” “heteroarylene,” or “heterocyclylene” are intended to mean an aryl, heteroaryl, or heterocyclyl group, respectively, as defined hereinabove, that is positioned between and serves to connect two other chemical groups.

Examples of heterocyclyls and heteroaryls include, but are not limited to, azepinyl, azetidinyl, acridinyl, azocinyl, benzidolyl, benzimidazolyl, benzofuranyl, benzofurazanyl, benzofuryl, benzothiofuranyl, benzothiophenyl, benzoxazolyl, benzothiazolyl, benzothienyl, benztriazolyl, benztetrazolyl, benzisoxazolyl, benzisothiazolyl, benzimidazolinyl, benzoxazolyl, benzoxadiazolyl, benzopyranyl, carbazolyl, 4aH-carbazolyl, carbolinyl, chromanyl, chromenyl, cinnolinyl, coumarinyl, decahydroquinolinyl, 1,3-dioxolane, 2H,6H-1,5,2-dithiazinyl, dihydrofuro[2,3-b]tetrahydrofuran, dihydroisoindolyl, dihydroquinazolinyl (such as 3,4-dihydro-4-oxo-quinazolinyl), furanyl, furopyridinyl (such as furo[2,3-c]pyridinyl, furo[3,2-b]pyridinyl or furo[2,3-b]pyridinyl), furyl, furazanyl, hexahydrodiazepinyl, imidazolidinyl, imidazolinyl, imidazolyl, indazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolidinyl, isothiazolyl, isoxazolinyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, oxetanyl, 2-oxoazepinyl, 2-oxopiperazinyl, 2-oxopiperidinyl, 2-oxopyrrolodinyl, pyrimidinyl, phenanthridinyl, phenanthrolinyl, phenazinyl, phenothiazinyl, phenoxathiinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolopyridyl, 2H-pyrrolyl, pyrrolyl, quinazolinyl, quinolinyl, 4H-quinolizinyl, quinoxalinyl, quinuclidinyl, tetrahydro-1,1-dioxothienyl, tetrahydrofuranyl, tetrahydrofuryl, tetrahydroisoquinolinyl, tetrahydroquinolinyl, tetrahydropyranyl, tetrazolyl, thiazolidinyl, 6H-1,2,5-thiadiazinyl, thiadiazolyl (e.g., 1,2,3-thiadiazolyl, 1,2,4-thiadiazolyl, 1,2,5-thiadiazolyl, 1,3,4-thiadiazolyl), thiamorpholinyl, thiamorpholinyl sulfoxide, thiamorpholuiyl sulfone, thianthrenyl, thiazolyl, thienyl, thienothiazolyl, thienooxazolyl, thienoimidazolyl, thiophenyl, triazinyl, triazinylazepinyl, triazolyl (e.g., 1,2,3-triazolyl, 1,2,4-triazolyl, 1,2,5-triazolyl, 1,3,4-triazolyl), and xanthenyl.

The term “azolyl” as employed herein is intended to mean a five-membered saturated or unsaturated heterocyclic group containing two or more hetero-atoms, as ring atoms, selected from the group consisting of nitrogen, sulfur and oxygen, wherein at least one of the hetero-atoms is a nitrogen atom. Examples of azolyl groups include, but are not limited to, optionally substituted imidazolyl, oxazolyl, thiazolyl, pyrazolyl, isoxazolyl, isothiazolyl, 1,3,4-thiadiazolyl, 1,2,4-thiadiazolyl, 1,2,4-oxadiazolyl, and 1,3,4-oxadiazolyl.

As employed herein, and unless stated otherwise, when a moiety (e.g., alkyl, heteroalkyl, cycloalkyl, aryl, heteroaryl, heterocyclyl, etc.) is described as “optionally substituted” it is meant that the group optionally has from one to four, alternatively from one to three, alternatively one or two, independently selected non-hydrogen substituents. Suitable substituents include, without limitation, halogen, hydroxy, oxo (e.g., an annular —CH— substituted with oxo is —C(O)—) nitro, halohydrocarbyl, hydrocarbyl, alkyl, cycloalkyl, heterocyclyl, aryl, heteroaryl, aralkyl, alkoxy, aryloxy, amino, acylamino, alkylcarbamoyl, arylcarbamoyl, aminoalkyl, acyl, carboxy, hydroxyalkyl, alkanesulfonyl, arenesulfonyl, alkanesulfonamido, arenesulfonamido, aralkylsulfonamido, alkylcarbonyl, acyloxy, cyano, and ureido groups.

Examples of substituents, which are themselves not further substituted (unless expressly stated otherwise) are:
(a) halogen, cyano, oxo, carboxy, formyl, nitro, amino, amidino, guanidino,
(b) C₁-C₅alkyl or alkenyl or arylalkyl imino, carbamoyl, azido, carboxamido, mercapto, hydroxy, hydroxyalkyl, alkylaryl, arylalkyl, C₁-C₈alkyl, C₂-C₈alkenyl, C₁-C₈alkoxy, C₁-C₈alkyamino, C₁-C₈alkoxycarbonyl, aryloxycarbonyl, C₂-C₈acyl, C₂-C₈acylamino, C₁-C₈alkylthio, arylalkylthio, arylthio, C₁-C₈alkylsulfinyl, arylalkylsulfinyl, arylsulfinyl, C₁-C₈alkylsulfonyl, arylalkylsulfonyl, arylsulfonyl, C₀-C₆N-alkyl carbamoyl, C₂-C₁₅N,N-dialkylcarbamoyl, C₃-C₇ cycloalkyl, aroyl, aryloxy, arylalkyl ether, aryl, aryl fused to a cycloalkyl or heterocycle or another aryl ring, C₃-C₇heterocycle, C₅-C₁₅heteroaryl or any of these rings fused or spiro-fused to a cycloalkyl, heterocyclyl, or aryl, wherein each of the foregoing is further optionally substituted with one more moieties listed in (a), above; and
(c) —(CR³²R³³)ₛ—NR³⁰R³¹,
   wherein s is from 0 (in which case the nitrogen is directly bonded to the moiety that is substituted) to 6,
   R³² and R³³ are each independently hydrogen, halogen, hydroxyl or C₁-C₄alkyl, and
   R³⁰ and R³¹ are each independently hydrogen, cyano, oxo, hydroxyl, C₁-C₈alkyl, C₁-C₈heteroalkyl, C₂-C₈alkenyl, carboxamido, C₁-C₃alkyl-carboxamido, carboxamido-C₁-C₃ alkyl, amidino, C₂-C₈hydroxyalkyl, C₁-C₃alkylaryl, aryl-C₁-C₃alkyl, C₁-C₃alkylheteroaryl, heteroaryl-C₁-C₃alkyl, C₁-C₃alkylheterocyclyl, heterocyclyl-C₁-C₃alkyl C₁-C₃alkylcycloalkyl, cycloalkyl-C₁-C₃alkyl, C₂-C₈alkoxy, C₂-C₈alkoxy-C₁-C₄alkyl, C₁-C₈alkoxycarbonyl, aryloxycarbonyl, aryl-C₁-C₃alkoxycarbonyl, heteroaryloxycarbonyl, heteroaryl-C₁-C₃alkoxycarbonyl, C₁-C₈acyl, C₀-C₈alkyl-carbonyl, aryl-C₀-C₈alkyl-carbonyl, heteroaryl-C₀-C₈alkyl-carbonyl, cycloalkyl-C₀-C₈alkyl-carbonyl, C₀-C₈alkyl-NH-carbonyl, aryl-C₀-C₈alkyl-NH-carbonyl, heteroaryl-C₀-C₈alkyl-NH-carbonyl, cycloalkyl-C₀-C₈alkyl-NH-carbonyl, C₀-C₈alkyl-O-carbonyl, aryl-C₀-C₈alkyl-O-carbonyl, heteroaryl-C₀-C₈alkyl-O-carbonyl, cycloalkyl-C₀-C₈alkyl-O-Carbonyl, C₁-C₈alkylsulfonyl, arylalkylsulfonyl, arylsulfonyl, heteroarylalkylsulfonyl, heteroarylsulfonyl, C₁-C₈alkyl-NH-sulfonyl, arylalkyl-NH-sulfonyl, aryl-NH-sulfonyl, heteroarylalkyl-NH-sulfonyl, heteroaryl-NH-sulfonyl aroyl, aryl, cycloalkyl, heterocyclyl, heteroaryl, aryl-C₁-C₃alkyl-, cycloalkyl-C₁-C₃alkyl-, heterocyclyl-C₁-C₃alkyl-, heteroaryl-C₁-C₃alkyl-, or protecting group, wherein each of the foregoing is further optionally substituted with one more moieties listed in (a), above; or
   R³⁰ and R³¹ taken together with the N to which they are attached form a heterocyclyl or heteroaryl, each of which is optionally substituted with from 1 to 3 substituents selected from the group consisting of (a) above, a protecting group, and (X³⁰—Y³¹—), wherein said heterocyclyl may also be bridged (forming a bicyclic moiety with a methylene, ethylene or propylene bridge); wherein
   X³⁰ is selected from the group consisting of C₁-C₈alkyl, C₂-C₈alkenyl-, C₂-C₈alkynyl-, —C₀-C₃alkyl-C₂-C₈alkenyl-C₀-C₃alkyl, C₀-C₃alkyl-C₂-C₈alkynyl-C₀-C₃alkyl, C₀-C₃alkyl-O—C₀-C₃alkyl-, HO—C₀-C₃alkyl-, C₀-C₄alkyl-N(R³⁰)—C₀-C₃alkyl-, N(R³⁰)(R³¹)—C₀-C₃alkyl-, N(R³⁰)(R³¹)—C₀-C₃alkenyl-, N(R³⁰)(R³¹)—C₀-C₃alkynyl-, (N(R³⁰)(R³¹))₂—C═N—, C₀-C₃alkyl-S(O)₀₋₂—C₀-C₃alkyl-, CF₃—C₀-C₃alkyl-, C₁-C₈heteroalkyl, aryl, cycloalkyl, heterocyclyl, heteroaryl, aryl-C₁-C₃alkyl-, cycloalkyl-C₁-C₃alkyl-, heterocyclyl-C₁-C₃alkyl-, heteroaryl-C₁-C₃alkyl-, N(R³⁰)(R³¹)-heterocyclyl-C₁-C₃alkyl-, wherein the aryl, cycloalkyl, heteroaryl and heterocyclyl are optionally substituted with from 1 to 3 substituents from (a); and
   Y³¹ is selected from the group consisting of a direct bond, —O—, —N(R³⁰)—, —C(O)—, —O—C(O)—, —C(O)—O—, —N(R³⁰)—C(O)—, —C(O)—N(R³⁰)—, —N(R³⁰)—C(S)—, —C(S)—N(R³⁰)—, —N(R³⁰)—C(O)—N(R³¹)—, —N(R³⁰)—C(NR³⁰)—N(R³¹)—, —N(R³⁰)—C(NR³¹)—, —C(NR³¹)—N(R³⁰)—, —N(R³⁰)—C(S)—N(R³¹)—, —N(R³⁰)—C(O)—O—, —O—C(O)—N(R³¹)—, —N(R³⁰)—C(S)—O—, —O—C(S)—N(R³¹)—, —S(O)₀₋₂—, —SO₂N(R³¹)—, —N(R³¹)—SO₂— and —N(R³⁰)—SO₂N(R³¹)—.

A moiety that is substituted is one in which one or more (for example one to four, alternatively from one to three and alternatively one or two), hydrogens have been independently replaced with another chemical substituent. As a non-limiting example, substituted phenyls include 2-fluorophenyl, 3,4-dichlorophenyl, 3-chloro-4-fluoro-phenyl, 2-fluoro-3-propylphenyl. As another non-limiting example, substituted n-octyls include 2,4-dimethyl-5-ethyl-octyl and 3-cyclopentyl-octyl. Included within this definition are methylenes (—CH₂—) substituted with oxygen to form carbonyl (—CO—).

When there are two optional substituents bonded to adjacent atoms of a ring structure, such as for example a phenyl, thiophenyl, or pyridinyl, the substituents, together with the atoms to which they are bonded, optionally form a 5- or 6-membered cycloalkyl or heterocycle having 1, 2, or 3 annular heteroatoms.

In some embodiments, a hydrocarbyl, heteroalkyl, heterocyclic and/or aryl group is unsubstituted.

In some embodiments, a hydrocarbyl, heteroalkyl, heterocyclic and/or aryl group is substituted with from 1 to 3 independently selected substituents.

Examples of substituents on alkyl groups include, but are not limited to, hydroxyl, halogen (e.g., a single halogen substituent or multiple halogen substituents; in the latter case, groups such as CF₃ or an alkyl group bearing Cl₃), oxo, cyano, nitro, alkyl, cycloalkyl, alkenyl, cycloalkenyl, alkynyl, heterocycle, aryl, —OR^{a}, —SR^{a}, —S(═O)R^{e}, —S(═O)₂R^{e}, —P(═O)₂R^{e}, —S(═O)₂OR^{e}, —P(═O)₂OR^{e}, —NR^{b}R^{c}, —NR^{b}S(═O)₂R^{e}, —NR^{b}P(═O)₂R^{e}, —S(═O)₂NR^{b}R^{c}, —P(═O)₂NR^{b}R^{c}, —C(═O)OR^{e}, —C(═O)R^{a}, —C(═O)NR^{b}R^{c}, —OC(═O)R^{a}, —OC(═O)NR^{b}R^{c}, —NR^{b}C(═O)OR^{e}, —NR^{d}C(═O)NR^{b}R^{c}, —NR^{d}S(═O)₂NR^{b}R^{c}, —NR^{d}P(═O)₂NR^{b}R^{c}, —NR^{b}C(═O)R^{a} or —NR^{b}P(═O)₂R^{e}, wherein R^{a} is hydrogen, alkyl, cycloalkyl, alkenyl, cycloalkenyl, alkynyl, heterocycle or aryl; R^{b}, R^{c} and R^{d} are independently hydrogen, alkyl, cycloalkyl, heterocycle or aryl, or said R^{b} and R^{c} together with the N to which they are bonded optionally form a heterocycle; and R^{e} is alkyl, cycloalkyl, alkenyl, cycloalkenyl, alkynyl, heterocycle or aryl. In the aforementioned exemplary substituents, groups such as alkyl, cycloalkyl, alkenyl, alkynyl, cycloalkenyl, heterocycle and aryl can themselves be optionally substituted.

Examples of substituents on alkenyl and alkynyl groups include, but are not limited to, alkyl or substituted alkyl, as well as those groups recited as examples of alkyl substituents.

Examples of substituents on cycloalkyl groups include, but are not limited to, nitro, cyano, alkyl or substituted alkyl, as well as those groups recited above as examples of alkyl substituents. Other examples of substituents include, but are not limited to, spiro-attached or fused cyclic substituents, for example, spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

Examples of substituents on cycloalkenyl groups include, but are not limited to, nitro, cyano, alkyl or substituted alkyl, as well as those groups recited as examples of alkyl substituents. Other examples of substituents include, but are not limited to, spiro-attached or fused cyclic substituents, for examples spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

Examples of substituents on aryl groups include, but are not limited to, nitro, cycloalkyl or substituted cycloalkyl, cycloalkenyl or substituted cycloalkenyl, cyano, alkyl or substituted alkyl, as well as those groups recited above as examples of alkyl substituents. Other examples of substituents include, but are not limited to, fused cyclic groups, such as fused cycloalkyl, fused cycloalkenyl, fused heterocycle, or fused aryl, where the aforementioned cycloalkyl, cycloalkenyl, heterocycle and aryl substituents can themselves be optionally substituted. Still other examples of substituents on aryl groups (phenyl, as a non-limiting example) include, but are not limited to, haloalkyl and those groups recited as examples of alkyl substituents.

Examples of substituents on heterocyclic groups include, but are not limited to, cycloalkyl, substituted cycloalkyl, cycloalkenyl, substituted cycloalkenyl, nitro, oxo (i.e., ═O), cyano, alkyl, substituted alkyl, as well as those groups recited as examples of alkyl substituents. Other examples of substituents on heterocyclic groups include, but are not limited to, spiro-attached or fused cyclic substituents at any available point or points of attachment, for example spiro-attached cycloalkyl, spiro-attached cycloalkenyl, spiro-attached heterocycle (excluding heteroaryl), fused cycloalkyl, fused cycloalkenyl, fused heterocycle and fused aryl, where the aforementioned cycloalkyl, cycloalkenyl, heterocycle and aryl substituents can themselves be optionally substituted.

In some embodiments, a heterocyclic group is substituted on carbon, nitrogen and/or sulfur at one or more positions. Examples of substituents on nitrogen include, but are not limited to alkyl, aryl, aralkyl, alkylcarbonyl, alkylsulfonyl, arylcarbonyl, arylsulfonyl, alkoxycarbonyl, or aralkoxycarbonyl. Examples of substituents on sulfur include, but are not limited to, oxo and C₁₋₆alkyl. In some embodiments, nitrogen and sulfur heteroatoms may independently be optionally oxidized and nitrogen heteroatoms may independently be optionally quaternized.

In some embodiments, substituents on ring groups, such as aryl, heteroaryl, cycloalkyl and heterocyclyl, include halogen, alkoxy and/or alkyl.

In some embodiments, substituents on alkyl groups include halogen and/or hydroxy.

A “halohydrocarbyl” as employed herein is a hydrocarbyl moiety, in which from one to all hydrogens have been replaced with halogen.

The term “halogen” or “halo” as employed herein refers to chlorine, bromine, fluorine, or iodine. As herein employed, the term “acyl” refers to an alkylcarbonyl or arylcarbonyl substituent. The term “acylamino” refers to an amide group attached at the nitrogen atom (i.e., R—CO—NH—). The term “carbamoyl” refers to an amide group attached at the carbonyl carbon atom (i.e., NH₂—CO—). The nitrogen atom of an acylamino or carbamoyl substituent is additionally optionally substituted. The term “sulfonamido” refers to a sulfonamide substituent attached by either the sulfur or the nitrogen atom. The term “amino” is meant to include NH₂, alkylamino, dialkylamino (wherein each alkyl may be the same or different), arylamino, and cyclic amino groups. The term “ureido” as employed herein refers to a substituted or unsubstituted urea moiety.

The term “radical” as used herein means a chemical moiety comprising one or more unpaired electrons.

Where optional substituents are chosen from “one or more” groups it is to be understood that this definition includes all substituents being chosen from within one of the specified groups or from within the combination of all of the specified groups.

In addition, substituents on cyclic moieties (i.e., cycloalkyl, heterocyclyl, aryl, heteroaryl) include 5- to 6-membered mono- and 9- to 14-membered bi-cyclic moieties fused to the parent cyclic moiety to form a bi- or tri-cyclic fused ring system. Substituents on cyclic moieties also include 5- to 6-membered mono- and 9- to 14-membered bi-cyclic moieties attached to the parent cyclic moiety by a covalent bond to form a bi- or tri-cyclic bi-ring system. For example, an optionally substituted phenyl includes, but is not limited to, the following:

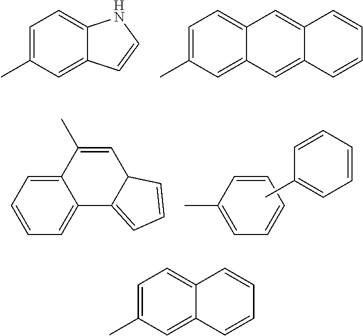

An “unsubstituted” moiety (e.g., unsubstituted cycloalkyl, unsubstituted heteroaryl, etc.) means a moiety as defined above that does not have any optional substituents.

A saturated, partially unsaturated or unsaturated three- to eight-membered carbocyclic ring is for example a four- to seven-membered, alternatively a five- or six-membered, saturated or unsaturated carbocyclic ring. Examples of saturated or unsaturated three- to eight-membered carbocyclic rings include phenyl, cyclopropyl, cyclobutyl, cyclopentyl, cyclohexyl, and cycloheptyl.

A saturated or unsaturated carbocyclic and heterocyclic group may condense with another saturated or heterocyclic group to form a bicyclic group, for example a saturated or unsaturated nine- to twelve-membered bicyclic carbocyclic or heterocyclic group. Bicyclic groups include naphthyl, quinolyl, 1,2,3,4-tetrahydroquinolyl, 1,4-benzoxanyl, indanyl, indolyl, and 1,2,3,4-tetrahydronaphthyl.

When a carbocyclic or heterocyclic group is substituted by two C₁-C₆alkyl groups, the two alkyl groups may combine together to form an alkylene chain, for example a C₁-C₃alkylene chain. Carbocyclic or heterocyclic groups having this crosslinked structure include bicyclo[2.2.2]octanyl and norbornanyl.

The terms “kinase inhibitor” and “inhibitor of kinase activity”, and the like, are used to identify a compound which is capable of interacting with a kinase and inhibiting its enzymatic activity.

The term “inhibiting kinase enzymatic activity” and the like is used to mean reducing the ability of a kinase to transfer a phosphate group from a donor molecule, such as adenosine tri-phosphate (ATP), to a specific target molecule (substrate). For example, the inhibition of kinase activity may be at least about 10%. In some embodiments of the invention, such reduction of kinase activity is at least about 25%, alternatively at least about 50%, alternatively at least about 75%, and alternatively at least about 90%. In other embodiments, kinase activity is reduced by at least 95% and alternatively by at least 99%. The IC₅₀ value is the concentration of kinase inhibitor which reduces the activity of a kinase to 50% of the uninhibited enzyme.

The terms “inhibitor of VEGF receptor signaling” is used to identify a compound having a structure as defined herein, which is capable of interacting with a VEGF receptor and inhibiting the activity of the VEGF receptor. In some embodiments, such reduction of activity is at least about 50%, alternatively at least about 75%, and alternatively at least about 90%. In some embodiments, activity is reduced by at least 95% and alternatively by at least 99%.

The term “inhibiting effective amount” is meant to denote a dosage sufficient to cause inhibition of kinase activity. The amount of a compound of the invention which constitutes an “inhibiting effective amount” will vary depending on the compound, the kinase, and the like. The inhibiting effective amount can be determined routinely by one of ordinary skill in the art. The kinase may be in a cell, which in turn may be in a multicellular organism. The multicellular organism may be, for example, a plant, a fungus or an animal, for example a mammal and for example a human. The fungus may be infecting a plant or a mammal, for example a human, and could therefore be located in and/or on the plant or mammal.

In an exemplary embodiment, such inhibition is specific, i.e., the kinase inhibitor reduces the ability of a kinase to transfer a phosphate group from a donor molecule, such as ATP, to a specific target molecule (substrate) at a concentration that is lower than the concentration of the inhibitor that is required to produce another, unrelated biological effect. For example, the concentration of the inhibitor required for kinase inhibitory activity is at least 2-fold lower, alternatively at least 5-fold lower, alternatively at least 10-fold lower, and alternatively at least 20-fold lower than the concentration required to produce an unrelated biological effect.

Thus, the invention provides a method for inhibiting kinase enzymatic activity, comprising contacting the kinase with an inhibiting effective amount of a compound or composition according to the invention. In some embodiments, the kinase is in an organism. Thus, the invention provides a method for inhibiting kinase enzymatic activity in an organism, comprising administering to the organism an inhibiting effective amount of a compound or composition according to the invention. In some embodiments, the organism is a mammal, for example a domesticated mammal. In some embodiments, the organism is a human.

The term “therapeutically effective amount” as employed herein is an amount of a compound of the invention, that when administered to a patient, elicits the desired therapeutic effect. The therapeutic effect is dependent upon the disease being treated and the results desired. As such, the therapeutic effect can be treatment of a disease-state. Further, the therapeutic effect can be inhibition of kinase activity. The amount of a compound of the invention which constitutes a “therapeutically effective amount” will vary depending on the compound, the disease state and its severity, the age of the patient to be treated, and the like. The therapeutically effective amount can be determined routinely by one of ordinary skill in the art.

In some embodiments, the therapeutic effect is inhibition of angiogenesis. The phrase “inhibition of angiogenesis” is used to denote an ability of a compound according to the present invention to retard the growth of blood vessels, such as blood vessels contacted with the inhibitor as compared to blood vessels not contacted. In some embodiments, angiogenesis is tumor angiogenesis. The phrase “tumor angiogenesis” is intended to mean the proliferation of blood vessels that penetrate into or otherwise contact a cancerous growth, such as a tumor. In some embodiments, angiogenesis is abnormal blood vessel formation in the eye.

In an exemplary embodiment, angiogenesis is retarded by at least 25% as compared to angiogenesis of non-contacted blood vessels, alternatively at least 50%, alternatively at least 75%, alternatively at least 90%, alternatively at least 95%, and alternatively, at least 99%. Alternatively, angiogenesis is inhibited by 100% (i.e., the blood vessels do not increase in size or number). In some embodiments, the phrase “inhibition of angiogenesis” includes regression in the number or size of blood vessels, as compared to non-contacted blood vessels. Thus, a compound according to the invention that inhibits angiogenesis may induce blood vessel growth retardation, blood vessel growth arrest, or induce regression of blood vessel growth.

Thus, the invention provides a method for inhibiting angiogenesis in an animal, comprising administering to an animal in need of such treatment a therapeutically effective amount of a compound or composition of the invention. In some embodiments, the animal is a mammal, for example a domesticated mammal. In some embodiments, the animal is a human.

In some embodiments, the therapeutic effect is treatment of an ophthalmic disease, disorder or condition. The phrase “treatment of an ophthalmic disease, disorder or condition” is intended to mean the ability of a compound according to the present invention to treat (a) a disease disorder or condition caused by choroidal angiogenesis, including, without limitation, age-related macular degeneration, or (b) diabetic retinopathy or retinal edema. In some embodiments the phrase “treatment of an ophthalmic disease, disorder or condition” is intended to mean the ability of a compound according to the present invention to treat an exudative and/or inflammatory ophthalmic disease, disorder or condition, a disorder related to impaired retinal vessel permeability and/or integrity, a disorder related to retinal microvessel rupture leading to focal hemorrhage, a disease of the back of the eye, a retinal disease, or a disease of the front of the eye, or other ophthalmic disease, disorder or condition.

In some embodiments, the ophthalmic disease, disorder or condition includes but is not limited to Age Related Macular Degeneration (ARMD), exudative macular degeneration (also known as “wet” or neovascular age-related macular degeneration (wet-AMD), macular oedema, aged disciform macular degeneration, cystoid macular oedema, palpebral oedema, retinal oedema, diabetic retinopathy, Acute Macular Neuroretinopathy, Central Serous Chorioretinopathy, chorioretinopathy, Choroidal Neovascularization, neovascular maculopathy, neovascular glaucoma, obstructive arterial and venous retinopathies (e.g. Retinal Venous Occlusion or Retinal Arterial Occlusion), Central Retinal Vein Occlusion, Disseminated Intravascular Coagulopathy, Branch Retinal Vein Occlusion, Hypertensive Fundus Changes, Ocular Ischemic Syndrome, Retinal Arterial Microaneurysms, Coat's Disease, Parafoveal Telangiectasis, Hemi-Retinal Vein Occlusion, Papillophlebitis, Central Retinal Artery Occlusion, Branch Retinal Artery Occlusion, Carotid Artery Disease (CAD), Frosted Branch Angitis, Sickle Cell Retinopathy and other Hemoglobinopathies, Angioid Streaks, macular oedema occurring as a result of aetiologies such as disease (e.g. Diabetic Macular Oedema), eye injury or eye surgery, retinal ischemia or degeneration produced for example by injury, trauma or tumours, uveitis, iritis, retinal vasculitis, endophthalmitis, panophthalmitis, metastatic ophthalmia, choroiditis, retinal pigment epithelitis, conjunctivitis, cyclitis, scleritis, episcleritis, optic neuritis, retrobulbar optic neuritis, keratitis, blepharitis, exudative retinal detachment, corneal ulcer, conjunctival ulcer, chronic nummular keratitis, Thygeson keratitis, progressive Mooren's ulcer, an ocular inflammatory disease caused by bacterial or viral infection or by an ophthalmic operation, an ocular inflammatory disease caused by a physical injury to the eye, and a symptom caused by an ocular inflammatory disease including itching, flare, oedema and ulcer, erythema, erythema exsudativum multiforme, erythema nodosum, erythema annulare, scleroedema, dermatitis, angioneurotic oedema, laryngeal oedema, glottic oedema, subglottic laryngitis, bronchitis, rhinitis, pharyngitis, sinusitis, laryngitis or otitis media.

In some embodiments, the ophthalmic disease, disorder or condition is (a) a disease disorder or condition caused by choroidal angiogenesis, including, without limitation, age-related macular degeneration, or (b) diabetic retinopathy or retinal edema.

In some embodiments, the ophthalmic disease, disorder or condition includes but is not limited to age-related macular degeneration, diabetic retinopathy, retinal edema, retinal vein occlusion, neovascular glaucoma, retinopathy of prematurity, pigmentary retinal degeneration, uveitis, corneal neovascularization or proliferative vitreoretinopathy.

In some embodiments, the ophthalmic disease, disorder or condition is age-related macular degeneration, diabetic retinopathy or retinal edema.

Thus, the invention provides a method for treating an ophthalmic disease, disorder or condition in an animal, comprising administering to an animal in need of such treatment a therapeutically effective amount of a compound or composition of the invention. In some embodiments, the animal is a mammal, for example a domesticated mammal. In some embodiments, the animal is a human.

In some embodiments, the therapeutic effect is inhibition of retinal neovascularization. The phrase “inhibition of retinal neovascularization” is intended to mean the ability of a compound according to the present invention to retard the growth of blood vessels in the eye, for example new blood vessels originating from retinal veins, for example, to retard the growth of new blood vessels originating from retinal veins and extending along the inner (vitreal) surface of the retina.

In an exemplary embodiment, retinal neovascularization is retarded by at least 25% as compared to retinal neovascularization of non-contacted blood vessels, alternatively at least 50%, alternatively at least 75%, alternatively at least 90%, alternatively at least 95%, and alternatively, at least 99%. Alternatively, retinal neovascularization is inhibited by 100% (i.e., the blood vessels do not increase in size or number). In some embodiments, the phrase “inhibition of retinal neovascularization” includes regression in the number or size of blood vessels, as compared to non-contacted blood vessels. Thus, a compound according to the invention that inhibits retinal neovascularization may induce blood vessel growth retardation, blood vessel growth arrest, or induce regression of blood vessel growth.

Thus, the invention provides a method for inhibiting retinal neovascularization in an animal, comprising administering to an animal in need of such treatment a therapeutically effective amount of a compound or composition of the invention. In some embodiments, the animal is a mammal, for example a domesticated mammal. In some embodiments, the animal is a human.

In some embodiments, the therapeutic effect is inhibition of cell proliferation. The phrase “inhibition of cell proliferation” is used to denote an ability of a compound according to the present invention to retard the growth of cells contacted with the inhibitor as compared to cells not contacted. An assessment of cell proliferation can be made by counting contacted and non-contacted cells using a Coulter Cell Counter (Coulter, Miami, Fla.) or a hemacytometer. Where the cells are in a solid growth (e.g., a solid tumor or organ), such an assessment of cell

In an exemplary embodiment, growth of cells contacted with the inhibitor is retarded by at least 25% as compared to growth of non-contacted cells, alternatively at least 50%, alternatively at least 75%, alternatively at least 90%, alternatively at least 95%, and alternatively, at least 99%. Alternatively, cell proliferation is inhibited by 100% (i.e., the contacted cells do not increase in number). In some embodiments, the phrase “inhibition of cell proliferation” includes a reduction in the number or size of contacted cells, as compared to non-contacted cells. Thus, a compound according to the invention that inhibits cell proliferation in a contacted cell may induce the contacted cell to undergo growth retardation, to undergo growth arrest, to undergo programmed cell death (i.e., to apoptose), or to undergo necrotic cell death.

In some embodiments, the contacted cell is a neoplastic cell. The term “neoplastic cell” is used to denote a cell that shows aberrant cell growth. In some embodiments, the aberrant cell growth of a neoplastic cell is increased cell growth. A neoplastic cell may be a hyperplastic cell, a cell that shows a lack of contact inhibition of growth in vitro, a benign tumor cell that is incapable of metastasis in vivo, or a cancer cell that is capable of metastasis in vivo and that may recur after attempted removal. The term “tumorigenesis” is used to denote the induction of cell proliferation that leads to the development of a neoplastic growth.

In some embodiments, the contacted cell is in an animal. Thus, the invention provides a method for treating a cell proliferative disease or condition in an animal, comprising administering to an animal in need of such treatment a therapeutically effective amount of a compound or composition of the invention. In some embodiments, the animal is a mammal, for example a domesticated mammal. In some embodiments, the animal is a human.

The term “cell proliferative disease or condition” is meant to refer to any condition characterized by aberrant cell growth, such as abnormally increased cellular proliferation. Examples of such cell proliferative diseases or conditions amenable to inhibition and treatment include, but are not limited to, cancer. Examples of particular types of cancer include, but are not limited to, breast cancer, lung cancer, colon cancer, rectal cancer, bladder cancer, prostate cancer, leukemia and renal cancer. In some embodiments, the invention provides a method for inhibiting neoplastic cell proliferation in an animal comprising administering to an animal having at least one neoplastic cell present in its body a therapeutically effective amount of a compound of the invention or a composition thereof.

The term “patient” as employed herein for the purposes of the present invention includes humans and other animals, for example mammals, and other organisms. Thus the compounds, compositions and methods of the present invention are applicable to both human therapy and veterinary applications. In some embodiments the patient is a mammal, for example a human.

The terms “treating”, “treatment”, or the like, as used herein cover the treatment of a disease-state in an organism, and includes at least one of: (i) preventing the disease-state from occurring, in particular, when such animal is predisposed to the disease-state but has not yet been diagnosed as having it; (ii) inhibiting the disease-state, i.e., partially or completely arresting its development; (iii) relieving the disease-state, i.e., causing regression of symptoms of the disease-state, or ameliorating a symptom of the disease; and (iv) reversal or regression of the disease-state, such as eliminating or curing of the disease. In some embodiments of the present invention the organism is an animal, for example a mammal, for example a primate, for example a human. As is known in the art, adjustments for systemic versus localized delivery, age, body weight, general health, sex, diet, time of administration, drug interaction, the severity of the condition, etc., may be necessary, and will be ascertainable with routine experimentation by one of ordinary skill in the art. In some embodiments, the terms “treating”, “treatment”, or the like, as used herein cover the treatment of a disease-state in an organism and includes at least one of (ii), (iii) and (iv) above.

Administration for non-ophthalmic diseases, disorders or conditions may be by any route, including, without limitation, parenteral, oral, sublingual, transdermal, topical, intranasal, intratracheal, or intrarectal. In some embodiments, compounds of the invention are administered intravenously in a hospital setting. In some embodiments, administration may be by the oral route.

Examples of routes of administration for ophthalmic diseases, disorders and conditions include but are not limited to, systemic, periocular, retrobulbar, intracanalicular, intravitral injection, topical (for example, eye drops), subconjunctival injection, subtenon, transcleral, intracameral, subretinal, electroporation, and sustained-release implant. Other routes of administration, other injection sites or other forms of administration for ophthalmic situations will be known or contemplated by one skilled in the art and are intended to be within the scope of the present invention.

In some embodiments of the present invention, routes of administration for ophthalmic diseases, disorders and conditions include topical, subconjunctival injection, intravitreal injection, or other ocular routes, systemically, or other methods known to one skilled in the art to a patient following ocular surgery.

In some other embodiments of the present invention, routes of administration for ophthalmic diseases, disorders and conditions include topical, intravitreal, transcleral, periocular, conjunctival, subtenon, intracameral, subretinal, subconjunctival, retrobulbar, or intracanalicular.

In some embodiments of the present invention, routes of administration for ophthalmic diseases, disorders and conditions include topical administration (for example, eye drops), systemic administration (for example, oral or intravenous), subconjunctival injection, periocular injection, intravitreal injection, and surgical implant for local delivery.

In some embodiments of the present invention, routes of administration for ophthalmic diseases, disorders and conditions include intravitreal injection, periocular injection, and sustained-release implant for local delivery.

In some embodiments of the present invention, an intraocular injection may be into the vitreous (intravitreal), under the conjunctiva (subconjunctival), behind the eye (retrobulbar), into the sclera, under the Capsule of Tenon (sub-Tenon), or may be in a depot form.

In some embodiments of the present invention, administration is local, including without limitation, topical, intravitreal, periorbital, intraocular, and other local administration to the eye, the ocular and/or periocular tissues and spaces, including without limitation, via a delivery device.

The compounds of the present invention form salts which are also within the scope of this invention.

The term “salt(s)”, as employed herein, denotes acidic and/or basic salts formed with inorganic and/or organic acids and bases. In addition, when a compound of the present invention contains both a basic moiety, such as but not limited to a pyridine or imidazole, and an acidic moiety such as but not limited to a carboxylic acid, zwitterions (“inner salts”) may be formed and are included within the term “salt(s)” as used herein. Pharmaceutically acceptable (i.e., non-toxic (exhibiting minimal or no undesired toxicological effects), physiologically acceptable) salts are preferred, although other salts are also useful, e.g., in isolation or purification steps which may be employed during preparation. Salts of the compounds of the invention may be formed, for example, by reacting a compound of the present invention with an amount of acid or base, such as an equivalent amount, in a medium such as one in which the salts precipitates or in an aqueous medium followed by lyophilization.

The compounds of the present invention which contain a basic moiety, such as but not limited to an amine or a pyridine or imidazole ring, may form salts with a variety of organic and inorganic acids. Examples of acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, hydroxyethanesulfanotes (e.g., 2-hydroxyethanesulfonates), lactates, maleates, methanesulfonates, naphthalenesulfonates (e.g., 2-naphthalenesulfonates), nicotinates, nitrates, oxalates, pectinates, persulfates, phenylpropionates (e.g., 3-phenylpropionates), phosphates, picrates, pivalates, propionates, salicylates, succinates, sulfates (such as those formed with sulfuric acid), sulfonates, tartrates, thiocyanates, toluenesulfonates such as tosylates, undecanoates, and the like.

The compounds of the present invention which contain an acidic moiety, such as but not limited to a carboxylic acid, may form salts with a variety of organic and inorganic bases. Examples of basic salts include ammonium salts, alkali metal salts such as sodium, lithium and potassium salts, alkaline earth metal salts such as calcium and magnesium salts, salts with organic bases (for example, organic amines) such as benzathines, dicyclohexylamines, hydrabamines (formed with N,N-bis(dehydroabietyl)ethylenediamine), N-methyl-D-glucamines, N-methyl-D-glycamides, t-butyl amines, and salts with amino acids such as arginine, lysine and the like. Basic nitrogen-containing groups may be quaternized with agents such as lower alkyl halides (e.g. methyl, ethyl, propyl and butyl chlorides, bromides and iodides), dialkyl sulfates (e.g. dimethyl, diethyl, dibuty and diamyl sulfates), long chain halides (e.g. decyl, lauryl, myristyl and stearyl chlorides, bromides and iodides), aralkyl halides (e.g. benzyl and phenethyl bromides), and others.

As used herein, the term “pharmaceutically acceptable salts” is intended to mean salts that retain the desired biological activity of the above-identified compounds and exhibit minimal or no undesired toxicological effects. Examples of such salts include, but are not limited to, salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, palmoic acid, alginic acid, polyglutamic acid, naphthalenesulfonic acid, naphthalenedisulfonic acid, methanesulfonic acid, p-toluenesulfonic acid and polygalacturonic acid. Other salts include pharmaceutically acceptable quaternary salts known by those skilled in the art, which specifically include the quaternary ammonium salt of the formula—NR+Z—, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

Another aspect of the invention provides compositions comprising a compound according to the present invention. For example, in some embodiments of the invention, a composition comprises a compound, or an N-oxide, hydrate, solvate, pharmaceutically acceptable salt, complex or prodrug of a compound according to the present invention present in at least about 30% enantiomeric or diastereomeric excess. In some embodiments of the invention, the compound, N-oxide, hydrate, solvate, pharmaceutically acceptable salt, complex or prodrug is present in at least about 50%, at least about 80%, or even at least about 90% enantiomeric or diastereomeric excess. In some embodiments of the invention, the compound, N-oxide, hydrate, solvate, pharmaceutically acceptable salt, complex or prodrug is present in at least about 95%, alternatively at least about 98% and alternatively at least about 99% enantiomeric or diastereomeric excess. In other embodiments of the invention, a compound, N-oxide, hydrate, solvate, pharmaceutically acceptable salt, complex or prodrug is present as a substantially racemic mixture.

Some compounds of the invention may have chiral centers and/or geometric isomeric centers (E- and Z-isomers), and it is to be understood that the invention encompasses all such optical, enantiomeric, diastereoisomeric and geometric isomers. The invention also comprises all tautomeric forms of the compounds disclosed herein. Where compounds of the invention include chiral centers, the invention encompasses the enantiomerically and/or diasteromerically pure isomers of such compounds, the enantiomerically and/or diastereomerically enriched mixtures of such compounds, and the racemic and scalemic mixtures of such compounds. For example, a composition may include a mixture of enantiomers or diastereomers of a compound of Formula (I) in at least about 30% diastereomeric or enantiomeric excess. In some embodiments of the invention, the compound is present in at least about 50% enantiomeric or diastereomeric excess, in at least about 80% enantiomeric or diastereomeric excess, or even in at least about 90% enantiomeric or diastereomeric excess. In some embodiments of the invention, the compound is present in at least about 95%, alternatively in at least about 98% enantiomeric or diastereomeric excess, and alternatively in at least about 99% enantiomeric or diastereomeric excess.

The chiral centers of the present invention may have the S or R configuration. The racemic forms can be resolved by physical methods, such as, for example, fractional crystallization, separation or crystallization of diastereomeric derivates or separation by chiral column chromatography. The individual optical isomers can be obtained either starting from chiral precursors/intermediates or from the racemates by any suitable method, including without limitation, conventional methods, such as, for example, salt formation with an optically active acid followed by crystallization.

The present invention also includes prodrugs of compounds of the invention. The term “prodrug” is intended to represent a compound covalently bonded to a carrier, which prodrug is capable of releasing the active ingredient when the prodrug is administered to a mammalian subject. Release of the active ingredient occurs in vivo. Prodrugs can be prepared by techniques known to one skilled in the art. These techniques generally modify appropriate functional groups in a given compound. These modified functional groups however regenerate original functional groups by routine manipulation or in vivo. Prodrugs of compounds of the invention include compounds wherein a hydroxy, amino, carboxylic, or a similar group is modified. Examples of prodrugs include, but are not limited to esters (e.g., acetate, formate, and benzoate derivatives), carbamates (e.g., N,N-dimethylaminocarbonyl) of hydroxy or amino functional groups in compounds of the present invention), amides (e.g., trifluoroacetylamino, acetylamino, and the like), and the like.

The compounds of the invention may be administered, for example, as is or as a prodrug, for example in the form of an in vivo hydrolyzable ester or in vivo hydrolyzable amide. An in vivo hydrolyzable ester of a compound of the invention containing a carboxy or hydroxy group is, for example, a pharmaceutically acceptable ester which is hydrolyzed in the human or animal body to produce the parent acid or alcohol. Suitable pharmaceutically acceptable esters for carboxy include C₁-C₆alkoxymethyl esters (e.g., methoxymethyl), C₁-C₆alkanoyloxymethyl esters (e.g., for example pivaloyloxymethyl), phthalidyl esters, C₃-C₈cycloalkoxycarbonyloxy-C₁-C₆alkyl esters (e.g., 1-cyclohexylcarbonyloxyethyl); 1,3-dioxolen-2-onylmethyl esters (e.g., 5-methyl-1,3-dioxolen-2-onylmethyl; and C₁-C₆alkoxycarbonyloxyethyl esters (e.g., 1-methoxycarbonyloxyethyl) and may be formed at any appropriate carboxy group in the compounds of this invention.

An in vivo hydrolyzable ester of a compound of the invention containing a hydroxy group includes inorganic esters such as phosphate esters and α-acyloxyalkyl ethers and related compounds which as a result of the in vivo hydrolysis of the ester breakdown to give the parent hydroxy group. Examples of α-acyloxyalkyl ethers include acetoxymethoxy and 2,2-dimethylpropionyloxy-methoxy. A selection of in vivo hydrolyzable ester forming groups for hydroxy include alkanoyl, benzoyl, phenylacetyl and substituted benzoyl and phenylacetyl, alkoxycarbonyl (to give alkyl carbonate esters), dialkylcarbamoyl and N—(N,N-dialkylaminoethyl)-N-alkylcarbamoyl (to give carbamates), N,N-dialkylaminoacetyl and carboxyacetyl. Examples of substituents on benzoyl include morpholino and piperazino linked from a ring nitrogen atom via a methylene group to the 3- or 4-position of the benzoyl ring. A suitable value for an in vivo hydrolyzable amide of a compound of the invention containing a carboxy group is, for example, a N—C₁-C₆alkyl or N,N-di-C₁-C₆alkyl amide such as N-methyl, N-ethyl, N-propyl, N,N-dimethyl, N-ethyl-N-methyl or N,N-diethyl amide.

Upon administration to a subject, the prodrug undergoes chemical conversion by metabolic or chemical processes to yield a compound of the present invention.

The present invention is also directed to solvates and hydrates of the compounds of the present invention. The term “solvate” refers to a molecular complex of a compound with one or more solvent molecules in a stoichiometric or non-stoichiometric amount. A molecular complex of a compound or moiety of a compound and a solvent can be stabilized by non-covalent intra-molecular forces such as, for example, electrostatic forces, van der Waals forces, or hydrogen bonds. Those skilled in the art of organic chemistry will appreciate that many organic compounds can form such complexes with solvents in which they are obtained, prepared or synthesized, or from which they are precipitated or crystallized. The term “hydrate” refers to a complex in which the one or more solvent molecules are water and includes monohydrates, hemi-hydrates, dihydrates, hexahydrates, and the like. The meaning of the words “solvate” and “hydrate” are well known to those skilled in the art. Techniques for the preparation of solvates are well established in the art (see, for example, Brittain, Polymorphism in Pharmaceutical solids. Marcel Dekker, New York, 1999; Hilfiker, Polymorphism in the Pharmaceutical Industry, Wiley, Weinheim, Germany, 2006).

In some embodiments of this aspect, the solvent is an inorganic solvent (for example, water). In some embodiments of this aspect, the solvent is an organic solvent (such as, but not limited to, alcohols, such as, without limitation, methanol, ethanol, isopropanol, and the like, acetic acid, ketones, esters, and the like). In certain embodiments, the solvent is one commonly used in the pharmaceutical art, is known to be innocuous to a recipient to which such solvate is administered (for example, water, ethanol, and the like) and in preferred embodiments, does not interfere with the biological activity of the solute.

Throughout the specification, embodiments of one or more chemical substituents are identified. Also encompassed are combinations of various embodiments. For example, the invention describes some embodiments of D in the compounds and describes some embodiments of group G. Thus, as an example, also contemplated as within the scope of the invention are compounds in which examples of D are as described and in which examples of group G are as described.

### Compounds

According to one aspect, the invention is directed to compounds having the Formula (I):

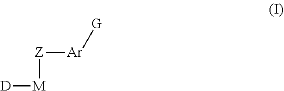

including N-oxides, hydrates, solvates, tautomers, pharmaceutically acceptable salts, prodrugs and complexes thereof, and racemic and scalemic mixtures, diastereomers and enantiomers thereof, wherein,
D is selected from the group consisting of an aromatic, heteroaromatic, cycloalkyl or heterocyclic ring system, C₁-C₆alkyl-heterocyclyl-C(O)—, C₁-C₆alkyl-heterocyclyl-C₁-C₆alkyl-N(R⁶)—C(O)—, (R⁶)(R⁶)N—C(O)—O-heterocyclyl-C(O)—, heterocyclyl-C(O)—, PivO-heterocyclyl-C(O)—, C₁-C₆alkyl-O—C(O)-heterocyclyl-C(O)—, C₁-C₆alkyl-C(O)—N(R⁶)— heterocyclyl-C(O)—, (C₁-C₆alkyl)(Boc)N-heterocyclyl-C(O)—, HO-heterocyclyl-C(O)—, HO—C(O)-heterocyclyl-C(O)—, C₁-C₆alkyl-C(O)—O-heterocyclyl-C(O)—, (R⁶)(R⁶)N—C₁-C₆alky-N(R⁶)—C(O)-heterocyclyl-C(O)—, C₁-C₆alkyl-heterocyclyl-C(O)-heterocyclyl-C(O)— and (R⁶)(R⁶)N-heterocyclyl-C(O)—, wherein each of the aromatic, heteroaromatic, cycloalkyl and heterocyclic groups is optionally substituted with 1 or more independently selected R³⁸;
M is an optionally substituted fused heterocyclic moiety;
Z is selected from the group consisting of —O—, —S(O)₀₋₂— and —NR⁵—, wherein R⁵ is selected from the group consisting of H, optionally substituted C₁-C₅alkyl, an optionally substituted (C₁-C₅)acyl and C₁-C₆ alkyl-O—C(O), wherein C₁-C₆ alkyl is optionally substituted;
Ar is a group of the formula C,

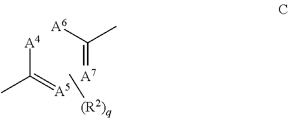

wherein,
A⁴, A⁵, A⁶ and A⁷ are independently selected from the group consisting of N and —CH—, with the proviso that no more than two of A⁴, A⁵, A⁶ and A⁷ can be N,
wherein Ar is optionally substituted with R²;
G is a group B-L-T, wherein
   B is selected from the group consisting of a covalent bond, —N(R¹³)—, —N(SO₂R¹³)—, —O—, —S(O)₀₋₂ and —C(═O)—;
   L is selected from the group consisting of a covalent bond, —C(═S)N(R¹³)—, —C(═NR¹⁴)N(R¹³)—, —SO₂N(R¹³)—, —SO₂—, —C(═O)N(R¹³)—, —N(R¹³)—, —C(═O)C₁₋₂alkyl-N(R¹³)—, —N(R¹³)C₁₋₂alkyl-C(═O)—, —C(═O)C₀₋₁alkyl-C(═O)N(R¹³)—, —C₀₋₄alkylene, —C(═O)C₀₋₁alkyl-C(═O)OR³—, —C(═NR¹⁴)—C₀₋₁alkyl-C(═O)—, —C(═O)—, —C(═O)C₀₋₁alkyl-C(═O)— and an optionally substituted four to six-membered heterocyclyl containing between one and three annular heteroatoms including at least one nitrogen, wherein the alkyl and alkylene are optionally substituted; and
   T is selected from the group consisting of —H, —R¹³, —C₀₋₄alkyl-O-Q, —N(R¹³)C₀₋₄alkyl-Q, —SO₂C₀₋₄alkyl-Q, —C(═O)C₀₋₄alkyl-Q, —C₀₋₄-alkyl-N(R¹³)Q and —C(═O)N(R¹³)—C₀₋₄alkyl-Q, wherein each C₀₋₄alkyl is optionally substituted;
wherein
R³⁸ is selected from the group consisting of R^{37b}O—C₁-C₆alkyl-C(O)-heterocyclyl-CH₂—, R³⁷O—(CH₂)ⱼ—[(CH₂)ᵢO]ₓ—(CH₂)ᵢ₁-heterocyclyl-CH₂—, heterocyclyl-CH₂—, R³⁷O—(CH₂)ⱼ—[(CH₂)ᵢO]ₓ—(CH₂)ⱼ—C(O)O—(CH₂)ᵢ₁—C(O)-heterocyclyl-CH₂—, R³⁷O—(CH₂)ⱼ-[(CH₂)ᵢO]ₓ—(CH₂)ᵢ₁—N(R²⁰¹)-heterocyclyl-CH₂—, R³⁷O—(CH₂)ⱼ-[(CH₂)ᵢO]ₓ—(CH₂)ᵢ₁—, R³⁷O—(CH₂)ⱼ-[(CH₂)ᵢO]ₓ—(CH₂)ᵢ₁—O-aryl-NHCH₂—, R³⁷O—(CH₂)ⱼ-[(CH₂)ᵢO]ₓ—(CH₂)—C(O)O—(CH₂)ᵢ₁—C(O)-heterocyclyl-N(R²⁰¹)CH₂—, R³⁷O—(CH₂)ⱼ-[(CH₂)ᵢO]ₓ—(CH₂)ᵢ₁—C(O)-heterocyclyl-CH₂—, R³⁷O—(CH₂)ⱼ-[(CH₂)ᵢO]ₓ—(CH₂)ᵢ₁—OC(O)-heterocyclyl-CH₂—, R³⁷O—(CH₂)ⱼ-[(CH₂)ᵢO]ₓ—(CH₂)ᵢ₁—NHC(O)-heterocyclyl-CH₂—, R³⁷O—(CH₂)ⱼ-[(CH₂)ᵢO]ₓ—(CH₂)ᵢ₁—N(R²⁰⁰)C(O)-heterocyclyl-CH₂—, R³⁷O—(CH₂)ⱼ-[(CH₂)ᵢO]ₓ—(CH₂)ᵢ₁—O-heterocyclyl-CH₂—, R³⁷O—(CH₂)ⱼ-[(CH₂)ᵢO]ₓ—(CH₂)—C(O)O—(CH₂)ᵢ₁-heterocyclyl-N(R²⁰¹)CH₂—;
each R⁶ is independently H or C₁-C₆alkyl;
R³⁷ is selected from the group consisting of H, C₁-C₆alkyl and C₃-C₁₀cycloalkyl;
R^{37b} is selected from the group consisting of (HO)₂P(═O)—, R²⁰¹HNCH(R²⁰⁰)C(O)NHCH(R²⁰⁰)C(O)—, R²⁰¹NHCH(R²⁰⁰)C(O)—, R²⁰¹CH(R²⁰⁰)CH(R²⁰⁰)C(O)—, R²⁰¹HNCH(R²⁰⁰)C(O)NHCH(R²⁰⁰)C(O)— and is HO—SO₂—;
j is an integer ranging from 0 to 4, alternatively 0 to 2;
i is 2 or 3;
x is an integer ranging from 0 to 6, alternatively 2 or 3;
i1 is 2 or 3;
R²⁰⁰ is selected from the group consisting of H, C₁-C₆-alkyl, C₃-C₆-cycloalkyl, aryl-(C₁-C₆-alkyl)-, OR²⁰¹, NHR²⁰¹ and SR²⁰¹; wherein the alkyl, aryl and cyclylalkyl moieties of the foregoing R²⁰⁰ groups are optionally substituted;
R²⁰¹ is selected from the group consisting of H, C₁-C₆-alkyl; aryl, aryl-(C₁-C₆-alkyl)-; (C₁-C₆aryl-(C₁-C₆-alkyl)C(O); (C₁-C₆-alkyl)OC(O); aryl-(C₁-C₆-alkyl)C(O); (C₁-C₆-alkyl)NHC(O); (C₁-C₆-alkyl)NHC(O)O—; (C₁-C₆-alkyl)NHC(O)NH—; (C₁-C₆)SO₂—, wherein the alkyl, and aryl moieties of the foregoing R²⁰¹ groups are optionally substituted;
R² at each occurrence is independently selected from the group consisting of —H, halogen, trihalomethyl, —CN, —NO₂, —NH₂, —OR³, —NR³R⁴, —S(O)₀₋₂R³, —S(O)₂NR³R³, —C(O)OR³, —C(O)NR³R³, —N(R³)SO₂R³, —N(R³)C(O)R³, —N(R³)CO₂R³, —C(O)R³, C₁-C₄ alkoxy, C₁-C₄ alkylthio, —O(CH₂)ₙaryl, —O(CH₂)ₙheteroaryl, —(CH₂)₀₋₅(aryl), —(CH₂)₀₋₅(heteroaryl), C₁-C₆ alkyl, C₂-C₆ alkenyl, C₂-C₆ alkynyl, —CH₂(CH₂)₀₋₄-T², wherein T² is selected from the group consisting of —OH, —OMe, —OEt, —NH₂, —NHMe, —NMe₂, —NHEt and —NEt₂, and wherein the aryl, heteroaryl, C₁-C₆ alkyl, C₂-C₆ alkenyl, and C₂-C₆ alkynyl are optionally substituted; and
q is an integer from 0 to 4;
n is an integer ranging from 0 to 4;
each R³ is independently selected from the group consisting of —H and R⁴;
R⁴ is selected from the group consisting of a (C₁-C₆)alkyl, an aryl, a lower arylalkyl, a heterocyclyl and a lower heterocyclyl-alkyl, each of which is optionally substituted, or
R³ and R⁴, taken together with a common nitrogen to which they are attached, form an optionally substituted five- to seven-membered heterocyclyl, the optionally substituted five- to seven-membered heterocyclyl optionally containing at least one additional annular heteroatom selected from the group consisting of N, O, S and P
R¹³ is selected from the group consisting of —H, —CN, —NO₂, —NH₂, —OR³, —NR³R⁴, —S(O)₀₋₂R³, —S(O)₂NR³R³, —C(O)OR³, —C(O)NR³R³, —N(R³)SO₂R³, —N(R³)C(O)R³, —N(R³)CO₂R³, —C(O)R³, —C(O)SR³, C1-C₄ alkoxy, C₁-C₄ alkylthio, —O(CH₂)ₙ₅aryl, —O(CH₂)ₙ₅heteroaryl, —(CH₂)ₙ₅(aryl), —(CH₂)ₙ₅(heteroaryl), C₁-C₆ alkyl, C₂-C₆ alkenyl, C₂-C₆ alkynyl, —CH₂(CH₂)₀₋₄-T², an optionally substituted C₁₋₄ alkylcarbonyl, and a saturated or unsaturated three- to seven-membered carboxyclic or heterocyclic group, wherein T² is selected from the group consisting of —OH, —OMe, —OEt, —NH₂, —NHMe, —NMe₂, —NHEt and —NEt₂, and wherein the aryl, heteroaryl, C₁-C₆ alkyl, C₂-C₆ alkenyl, and C₂-C₆ alkynyl are optionally substituted;
two R¹³, together with the atom or atoms to which they are attached, can combine to form a heteroalicyclic optionally substituted with between one and four of R⁶⁰, wherein the heteroalicyclic can have up to four annular heteroatoms, and the heteroalicyclic can have an aryl or heteroaryl fused thereto, in which case the aryl or heteroaryl is optionally substituted with an additional one to four of R⁶⁰;
n5 is an integer ranging from 0 to 6
R⁶⁰ is selected from the group consisting of —H, halogen, trihalomethyl, —CN, —NO₂, —NH₂, —OR³, —NR³R⁴, —S(O)₀₋₂R³, —SO₂NR³R³, —CO₂R³, —C(O)NR³R³, —N(R³)SO₂R³, —N(R³)C(O)R³, —N(R³)CO₂R³, —C(O)R³, an optionally substituted (C₁-C₆)alkyl, an optionally substituted aryl, an optionally substituted heteroarylalkyl and an optionally substituted arylalkyl;
two R⁶⁰, when attached to a non-aromatic carbon, can be oxo;
R¹⁴ is selected from the group —H, —NO₂, —NH₂, —N(R³)R⁴, —CN, —OR³, an optionally substituted (C₁-C₆)alkyl, an optionally substituted heteroalicyclyl-alkyl, an optionally substituted aryl, an optionally substituted arylalkyl and an optionally substituted heteroalicyclic,
Q is a three- to ten-membered ring system, optionally substituted with zero, one or more of R²⁰;
R²⁰ is selected from the group consisting of —H, halogen, trihalomethyl, —CN, —NO₂, —NH₂, —OR³, —OCF₃, —NR³R⁴, —S(O)₀₋₂R³, —S(O)₂NR³R³, —C(O)OR³, —C(O)NR³R³, —N(R³)SO₂R³, —N(R³)C(O)R³, —N(R³)C(O)OR³, —C(O)R³, —C(O)SR³, C₁-C₄ alkoxy, C₁-C₄ alkylthio, —O(CH₂)ₙ₆aryl, —O(CH₂)ₙ₆heteroaryl, —(CH₂)ₙ₆(aryl), —(CH₂)ₙ₆(heteroaryl), C₁-C₆ alkyl, C₂-C₆ alkenyl, C₂-C₆ alkynyl, —CH₂(CH₂)₀₋₄-T², an optionally substituted C₁₋₄ alkylcarbonyl, C₁₋₄ alkoxy, an amino optionally substituted by C₁₋₄ alkyl optionally substituted by C₁₋₄ alkoxy, —(CH₂)ₙ₆P(═O)(C₁-C₆alkyl)₂, a saturated or unsaturated three- to seven-membered carboxyclic or heterocyclic group, —SiMe₃ and —SbF₅; and
n6 is an integer ranging from 0 to 6.

In some embodiments of the first aspect, the compounds have the Formula (I), wherein D is -aryl or -heteroaryl each of which is substituted with 1 or more R³⁸.

In some embodiments of the first aspect, the compounds have the Formula (I), wherein D is selected from the group consisting of

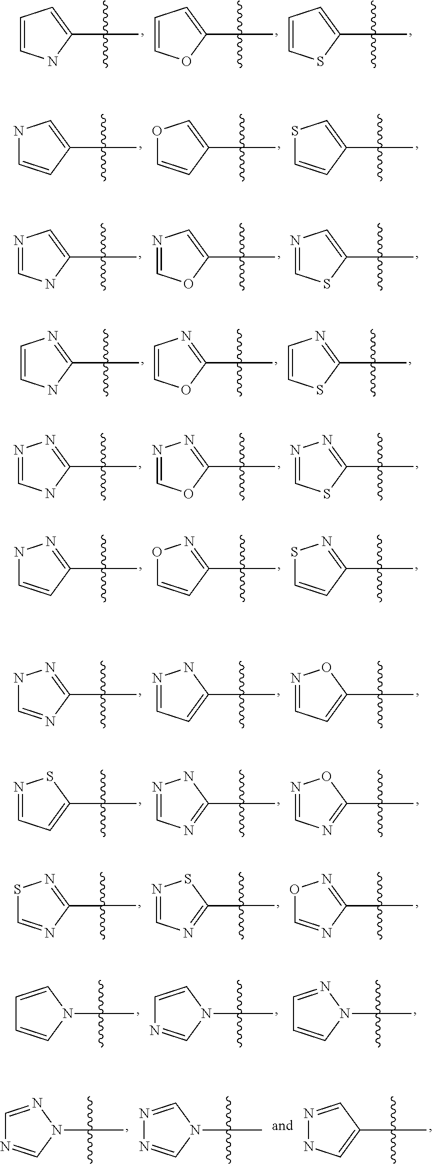

wherein the members of said group are substituted by 1 or more R³⁸.

In some embodiments of the first aspect, the compounds have the Formula (I), wherein D is selected from the group consisting of

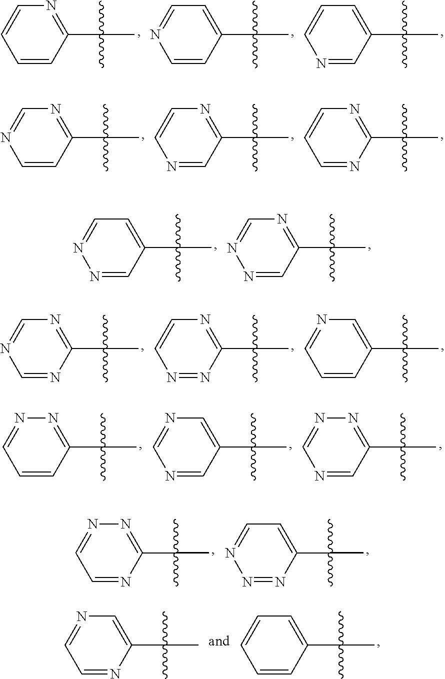

wherein the members of said group are substituted with 1 or more R³⁸.

In some embodiments of the first aspect, the compounds have the Formula (I), wherein D is selected from the group consisting of triazinyl, pyridinyl, imidazolyl, thiazolyl, pyrazolyl and phenyl substituted with one R³⁸, wherein when D is imidazolyl said imidazolyl is further optionally substituted with one C₁-C₆alkyl. In some embodiments of the first aspect, the compounds have the Formula (I), wherein D is phenyl or pyridine substituted with one R³⁸.

In some embodiments of the first aspect, the compounds have the Formula (I), wherein M is a structure selected from the group consisting of

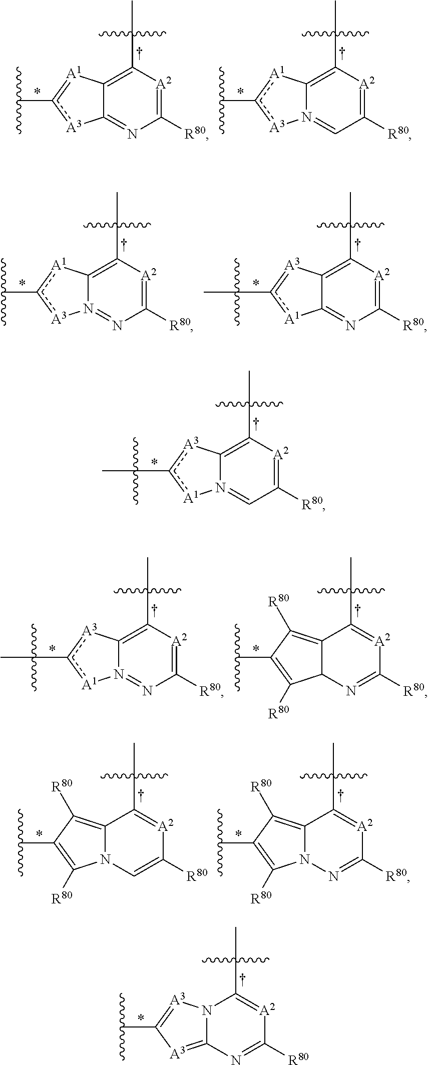

wherein
* represents the point of attachment to D;
† represents the point of attachment to Z;
A¹ is selected from the group consisting of CH, —O—, —S—, —N(H)—, —N(C₁-C₆ alkyl)-, —N—(Y-aryl)-, —N-OMe, —NCH₂OMe and N-Bn;
Y is a bond or —(C(R^{x})(H))ₜ—, wherein t is an integer from 1 to 6; and
R^{x} at each occurrence is independently selected from the group consisting of H and C₁-C₆ alkyl, wherein the C₁-C₆ alkyl is optionally substituted;
A² is selected from the group consisting of N and CR, wherein R is selected from the group consisting of —H, halogen, —CN, C₁-C₆ alkyl, C₂-C₆ alkenyl, C₂-C₆ alkynyl, —COOH and —C(O)Oalkyl, wherein the C₁-C₆ alkyl, C₂-C₆ alkenyl, C₂-C₆ alkynyl and —C(O)Oalkyl are optionally substituted;
each A³ is independently selected from the group consisting of CH and N;
each R⁸⁰ is independently selected from the group consisting of H, halogen, NO₂, cyano, OR⁸³, N(R⁸³)₂, CO₂R⁸³, C(O)N(R⁸³)₂, SO₂R⁸³, SO₂N(R⁸³)₂, NR⁸³SO₂R⁸³, NR⁸³C(O)R⁸³, NR⁸³CO₂R⁸³, —CO(CH₂)₁R⁸³, —CONH(CH₂)₁R⁸³, alkylaminoalkyl, alkylaminoalkynyl, C₁-C₆alkyl, substituted C₁-C₆alkyl, C₃-C₇cycloalkyl, substituted C₃-C₇cycloalkyl, alkenyl, substituted alkenyl, alkynyl, substituted alkynyl, hydroxyalkyl, aryl, substituted aryl, heteroaryl, substituted heteroaryl, arylalkyl, substituted arylalkyl, heterocycloalkyl, and substituted heterocycloalkyl; and
each R⁸³ is independently selected from the group consisting of H, alkyl, substituted alkyl, cycloalkyl, substituted cycloalkyl, aryl, substituted aryl, arylalkyl, substituted arylalkyl, heteroaryl, substituted heteroaryl, heterocycloalkyl, and substituted heterocycloalkyl; or
two R⁸³ taken together with the N atom to which they are attached form a heterocyclic ring.

In some embodiments of the first aspect, the compounds have the Formula (I), wherein M is a structure selected from the group consisting of

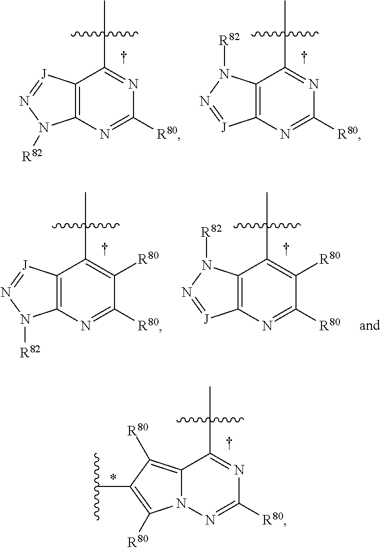

wherein
J is CR⁸⁰ or N;
R⁸² is selected from the group consisting of H, C₁-C₆alkyl or substituted C₁-C₆alkyl, —Y-(aryl), —Y-(heteroaryl), -alkoxy and —CH₂OMe;
wherein *, †, R⁸⁰ and Y are as defined above.

In some embodiments of the first aspect, the compounds have the Formula (I), wherein M is a structure selected from the group consisting of

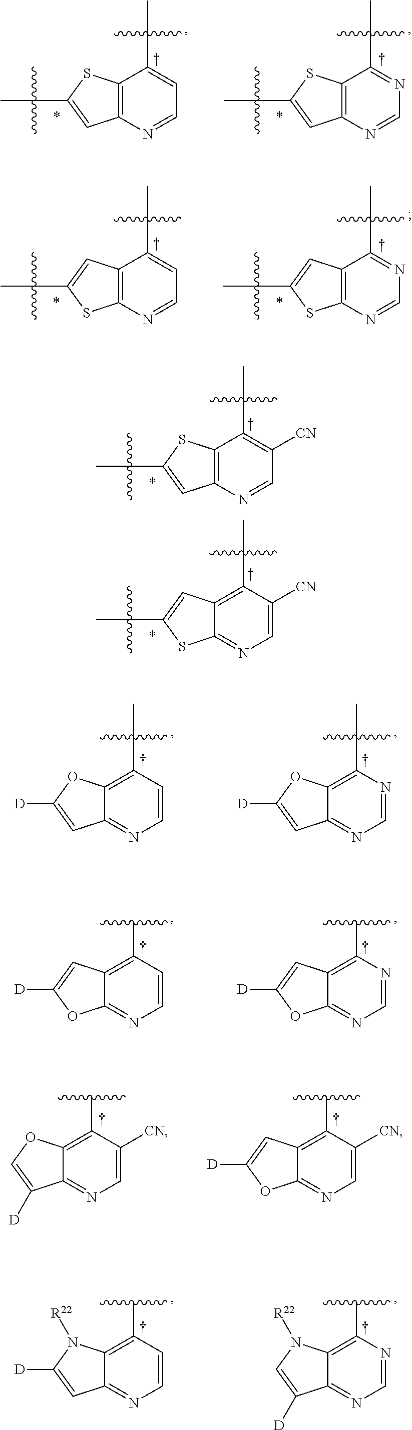

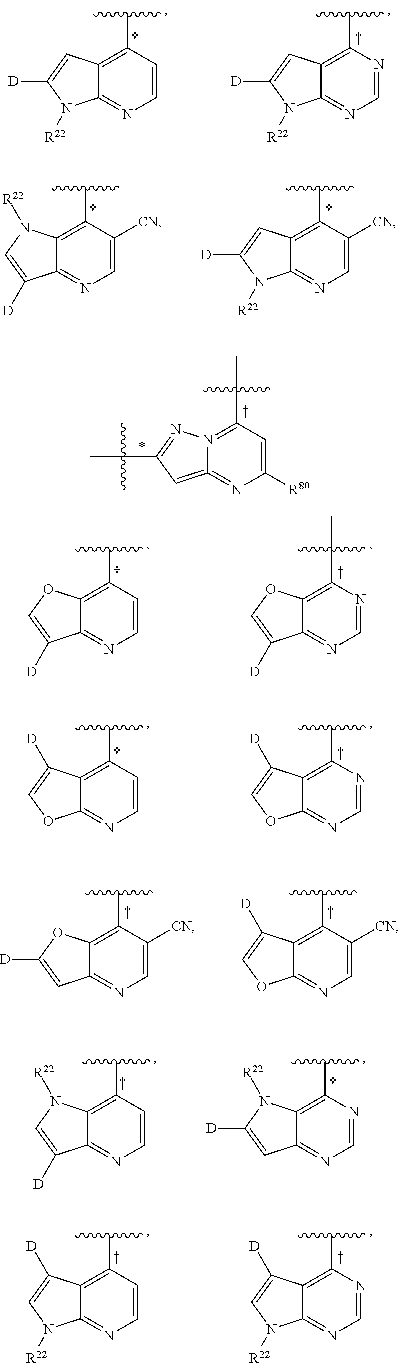

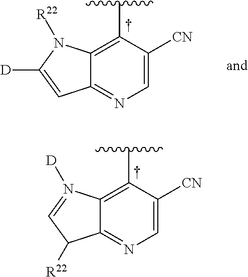

wherein
† is as defined above; and
R²² is selected from the group consisting of —H, —C₁-C₆alkyl, —Y-aryl, alkoxy, —CH₂—O-Me and —Bn.

In some embodiments of the first aspect, the compounds have the Formula (I), wherein M is

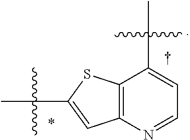 [SMILES: *c1cc2nccc(*)c2s1]

In some embodiments of the first aspect, the compounds have the Formula (I), wherein Z is O.

In some embodiments of the first aspect, the compounds have the Formula (I), wherein Ar is selected from the group consisting of phenyl, pyrazine, pyridazine, pyrimidine and pyridine, wherein each of said phenyl, pyrazine, pyridazine, pyrimidine and pyridine are optionally substituted with between zero and four R².

In some embodiments of the first aspect, the compound have the Formula (I), wherein Ar is phenyl, optionally substituted with between zero and four R².

In some embodiments of the first aspect, the compounds have the Formula (I), wherein Ar is phenyl, substituted with between zero and four halogen.

In some embodiments of the first aspect, the compounds have the Formula (I), wherein G is selected from the group consisting of

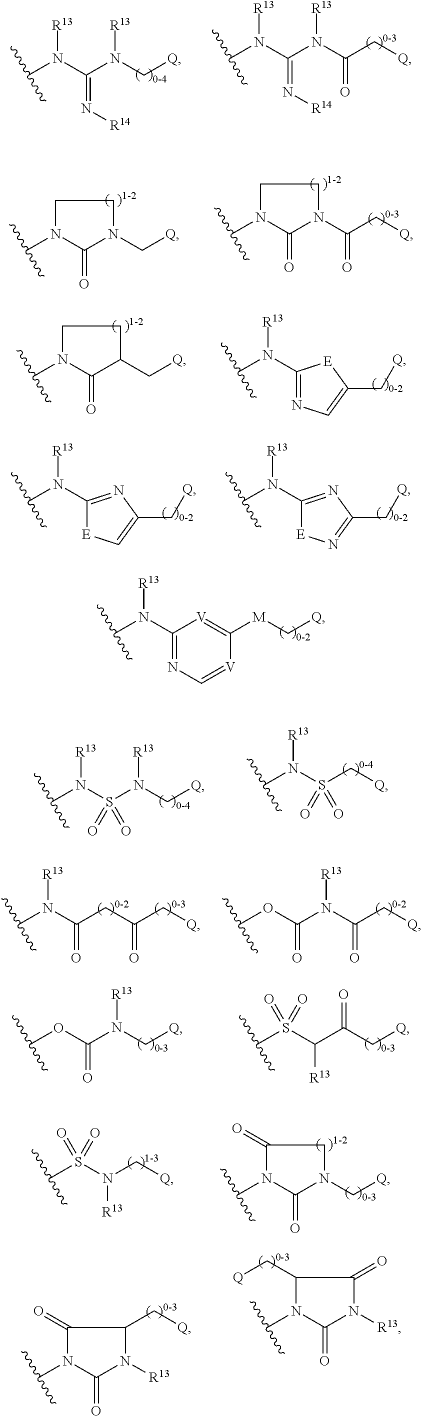

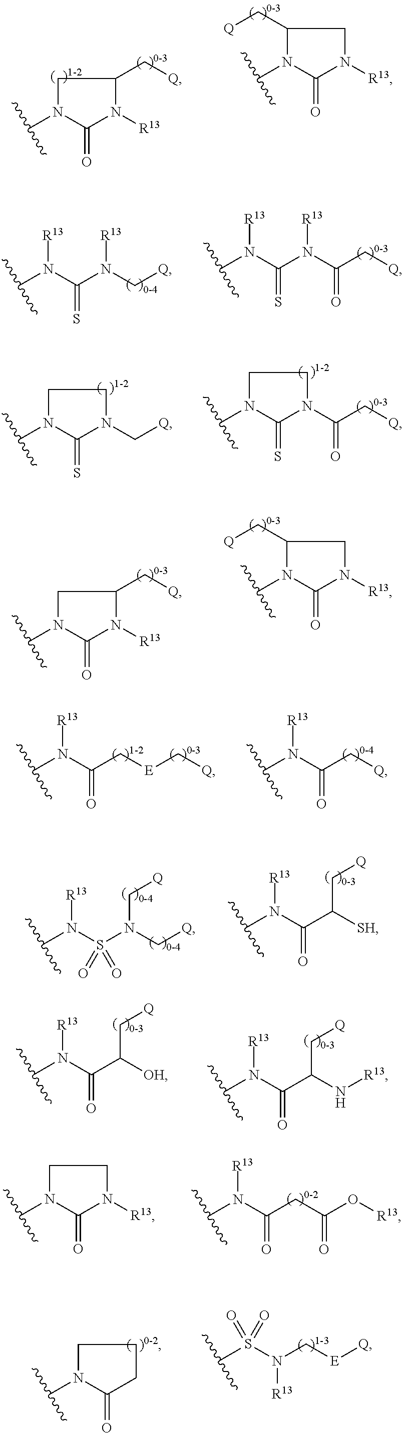

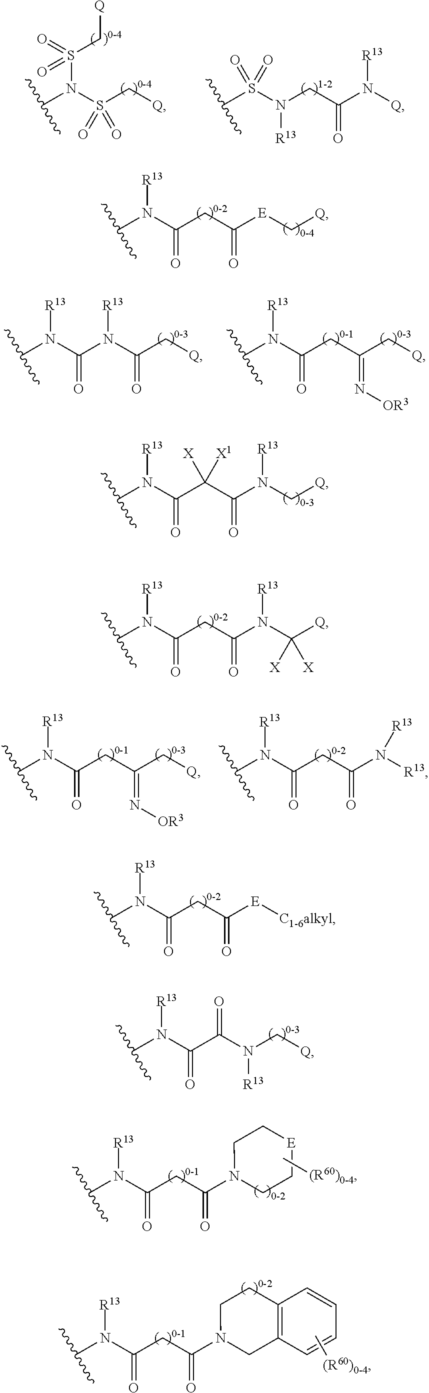

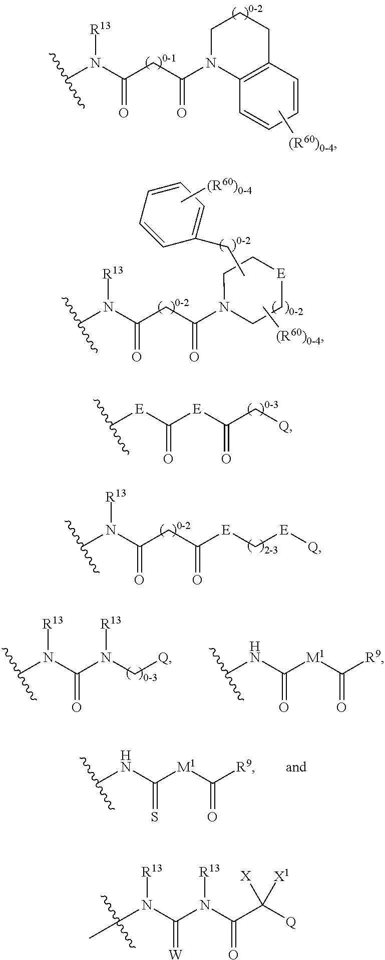

wherein R¹³, R¹⁴, Q, R³ and R⁴ are as defined above;
W is S, O or NH;
any methylene group is independently optionally substituted with R²⁵, wherein
R²⁵ is selected from the group consisting of halogen, trihalomethyl, —CN, —NO₂, —NH₂, —OR³, —NR³R⁴, —S(O)₀₋₂R³, —SO₂NR³R³, —CO₂R³, —C(O)NR³R³, —N(R³)SO₂R³, —N(R³)C(O)R³, —N(R³)CO₂R³, —C(O)R³, an optionally substituted aryl, an optionally substituted arylalkyl, an optionally substituted heteroarylalkyl, and an optionally substituted (C₁-C₆)alkyl,
two R²⁵, together with the carbon or carbons to which they are attached, can combine to form a three- to seven-membered alicyclic or heteroalicyclic, and
two R²⁵, on a single carbon can be oxo;
R⁹ is selected from the group consisting of a C₁₋₆ alkyl on which one or more hydrogen atoms are optionally substituted by —R²¹, -T¹-R¹⁵, or —NR¹⁶R¹⁷, a —N(R¹⁸)(R¹⁹) moiety and a saturated or unsaturated three- to eight-membered carbocyclic or heterocyclic group which is optionally substituted by a C₁₋₆ alkyl, a C₁₋₆ alkoxy, a halogen atom, nitro, a trifluoromethyl, a C₁₋₆ alkoxy carbonyl, cyano, a cyano C₁₋₆ alkyl, a C₁₋₆ alkylthio, a phenoxy, an acetyl, or a saturated or unsaturated five- or six-membered heterocyclyl ring wherein, when the three- to eight-membered carbocyclic or heterocyclic group is substituted by two C₁₋₆ alkyl groups, the two alkyl groups may combine together to form an alkylene chain, or the three- to eight-membered carbocyclic or heterocyclic group may be a bicyclic group condensed with another saturated or unsaturated three- to eight-membered carbocyclic or heterocyclic group,
wherein
T¹ is selected from the group consisting of —O—, —S— and —NH—;
R²¹ represents a saturated or unsaturated three- to eight-membered carbocyclic or heterocyclic group;
R¹⁵, R¹⁶, and R¹⁷, which may be the same or different, represent a C₁₋₆ alkyl or a saturated or unsaturated three- to eight-membered carbocyclic or heterocyclic group; wherein the three- to eight-membered carbocyclic or heterocyclic group represented by R²¹, R¹⁵, R¹⁶, and R¹⁷ is optionally substituted by a C₁₋₆ alkyl, a C₁₋₆ alkoxy, a halogen atom, nitro, a trifluoromethyl, a C₁₋₆ alkoxy carbonyl, a cyano, a cyano C₁₋₆ alkyl, a C₁₋₆ alkylthio, a phenoxy, an acetyl, or a saturated or unsaturated five- or six-membered heterocyclyl ring; and wherein when the three- to eight-membered carbocyclic or heterocyclic group is substituted by two C₁₋₆ alkyl groups, the two alkyl groups may combine together to form an alkylene chain; and wherein the three- to eight-membered carbocyclic or heterocyclic group may be a bicyclic group condensed with another saturated or unsaturated three- to eight-membered carbocyclic or heterocyclic group; and
R¹⁸ and R¹⁹, which may be the same or different, represent (1) a hydrogen atom, (2) a C₁₋₆ alkyl which is optionally substituted by a C₁₋₆ alkoxy, a C₁₋₆ alkylthio, or a saturated or unsaturated three- to eight-membered carbocyclic or heterocyclic group in which the three- to eight-membered carbocyclic or heterocyclic group is optionally substituted by a C₁₋₆ alkyl, a C₁₋₆ alkoxy, a halogen atom, nitro, a trifluoromethyl, a C₁₋₆ alkoxy carbonyl, cyano, a cyano C₁₋₆ alkyl, a C₁₋₆ alkylthio, a phenoxy, an acetyl, or a saturated or unsaturated five- or six-membered heterocyclyl ring and wherein when the three- to eight-membered carbocyclic or heterocyclic group is substituted by two C₁₋₆ alkyl groups, the two alkyl groups may combine together to form an alkylene chain, or the three- to eight-membered carbocyclic or heterocyclic group may be a bicyclic group condensed with another saturated or unsaturated three- to eight-membered carbocyclic or heterocyclic group, or (3) a saturated or unsaturated three- to eight-membered carbocyclic or heterocyclic group which is optionally substituted by a C₁₋₆ alkyl, a C₁₋₆ alkoxy, a halogen atom, nitro, a trifluoromethyl, a C₁₋₆ alkoxy carbonyl, cyano, a cyano C₁₋₆ alkyl, a C₁₋₆ alkylthio, a phenoxy, an acetyl, or a saturated or unsaturated five- or six-membered heterocyclyl ring and in which, when the three to eight-membered carbocyclic or heterocyclic group is substituted by two C₁₋₆ alkyl groups, the two alkyl groups may combine together to form an alkylene chain, or the three- to eight-membered carbocyclic or heterocyclic group may be a bicyclic group condensed with another saturated or unsaturated three- to eight-membered carbocyclic or heterocyclic group;
X and X¹ are each independently selected from the group consisting of —H, halogen, cyano, nitro, C₁-C₆ alkyl, or
X and X¹ together with the atom to which they are attached form a C₃-C₄ cycloalkyl;
E is selected from the group consisting of —O—, —N(R¹³)—, —CH₂— and —S(O)₀₋₂—;
M is selected from the group consisting of —O—, —N(R¹³)—, —CH₂— and —C(═O)N(R¹³);
M¹ represents —C(R²⁶)(R²⁷)—, wherein
R²⁶ and R²⁷ are independently selected from the group consisting of a hydrogen atom, a C₁₋₄ alkyl, a C₁₋₄ alkoxy and —N(R¹²), wherein
R¹² is a hydrogen atom or a C₁₋₄ alkyl; and
each V is independently selected from the group consisting of ═N— and ═C(H)—.

In some embodiments of the first aspect, the compounds have the Formula (I), wherein G is selected from the group consisting of

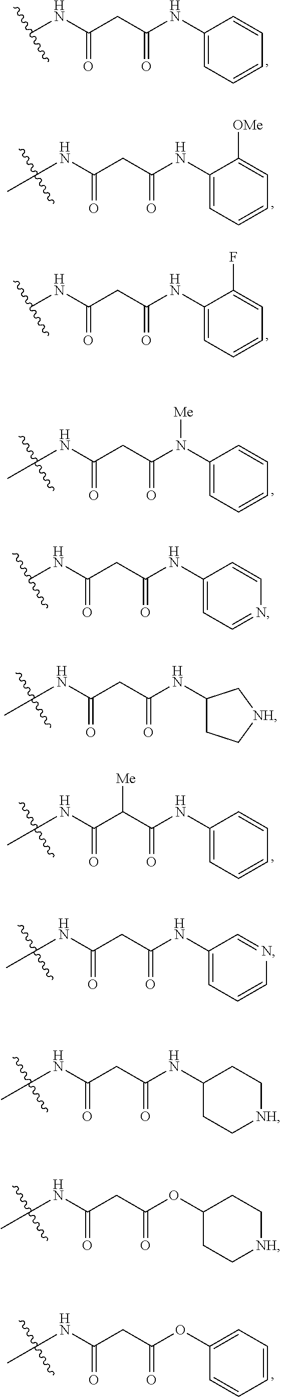

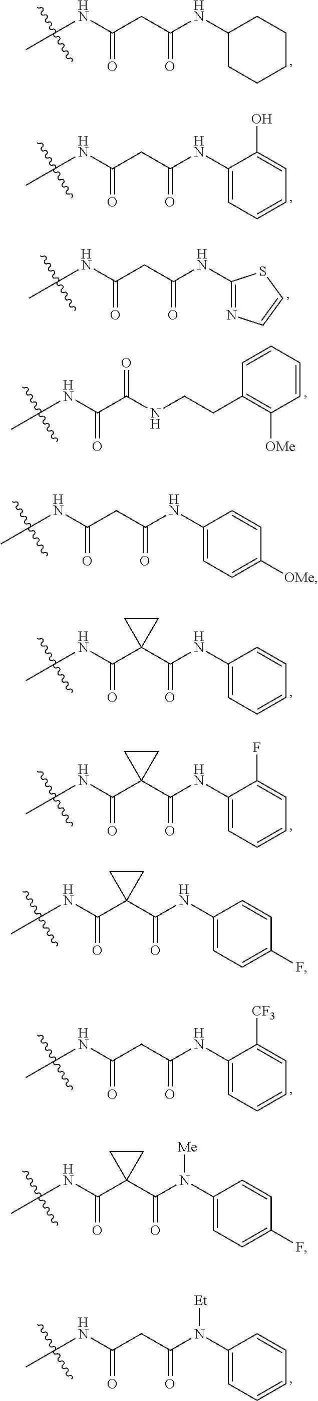

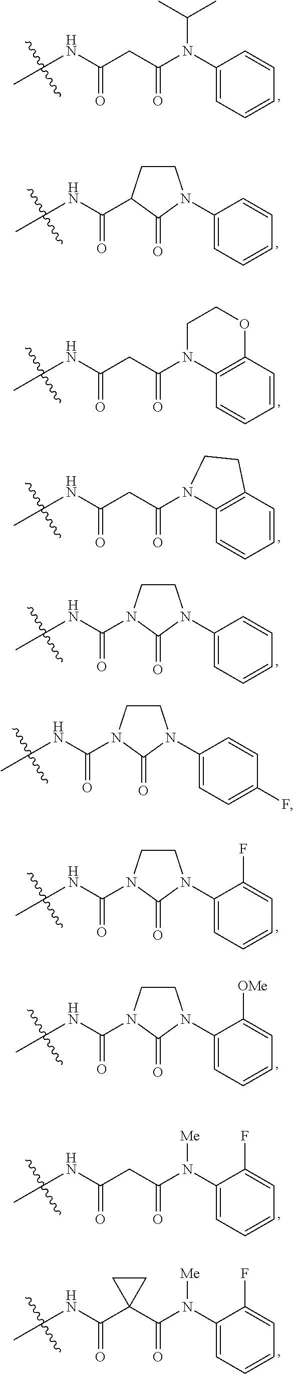

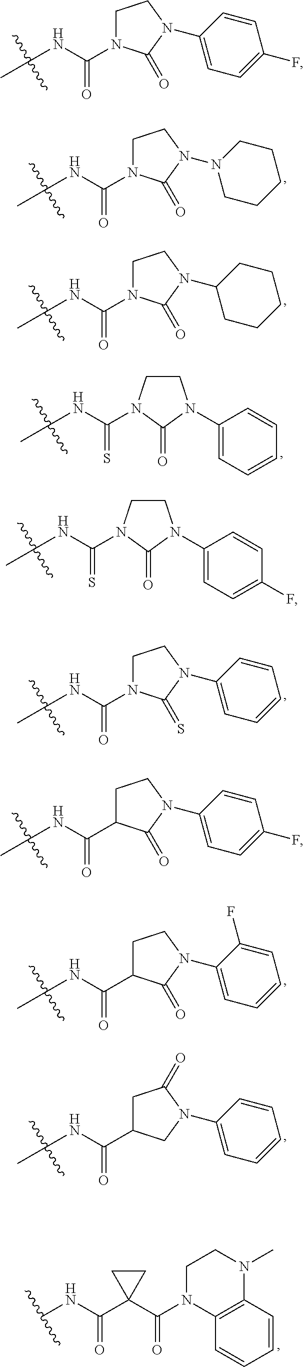

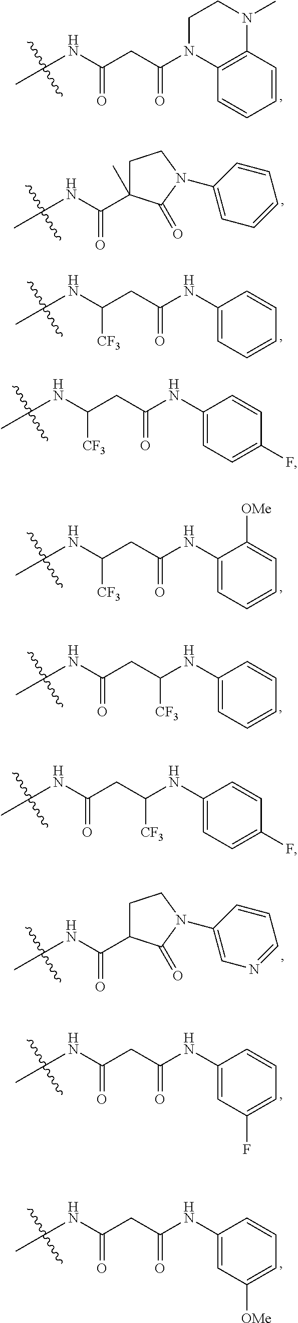

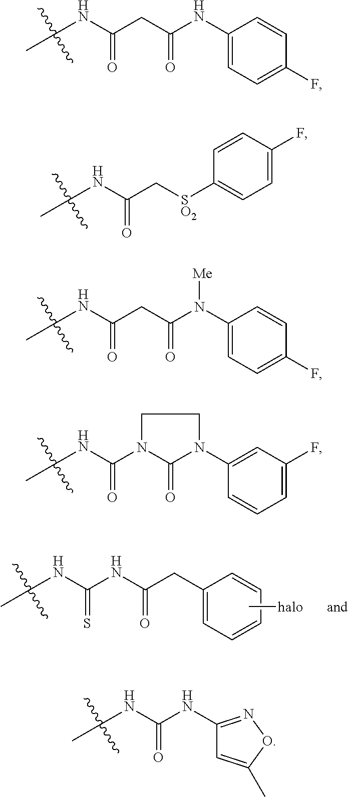

In some embodiments of the first aspect, the compounds have the Formula (I), wherein G is selected from the group consisting of

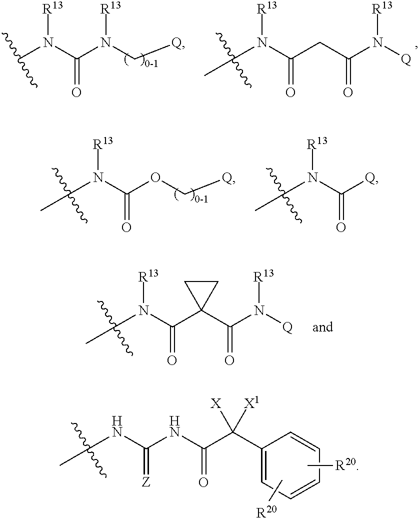

In some embodiments of the first aspect, the compounds have the Formula (I), wherein G is selected from the group consisting of

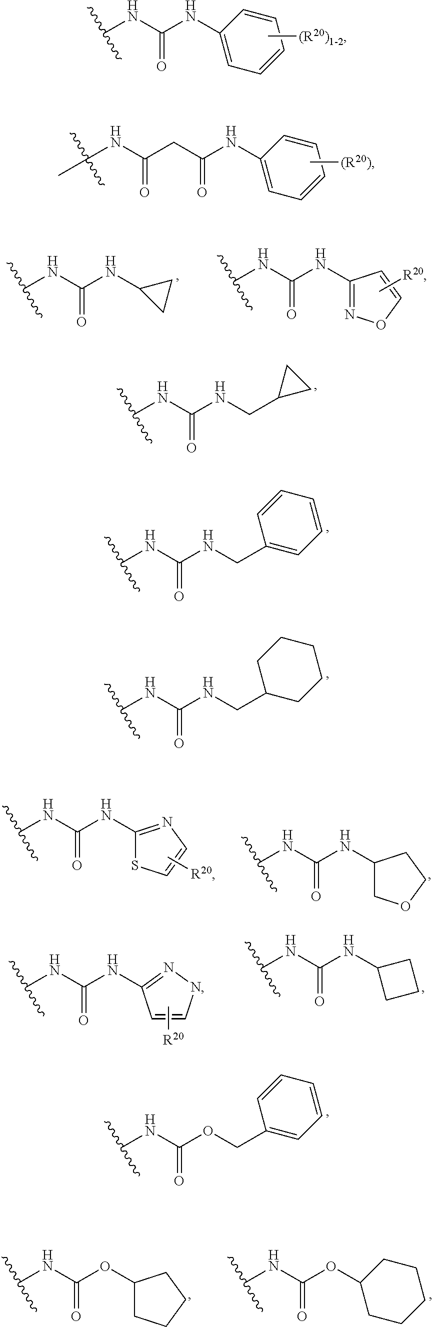

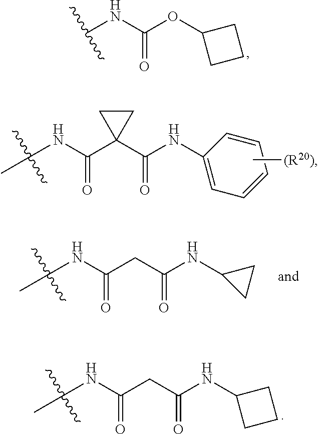

In some embodiments of the first aspect, the compounds have the Formula (I), wherein G is selected from the group consisting of

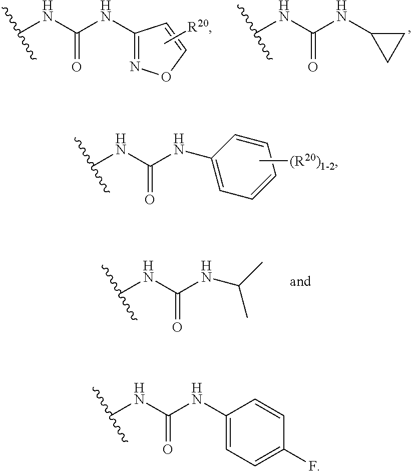

In some embodiments of the first aspect, the compounds have the Formula (I), wherein G is selected from the group consisting of

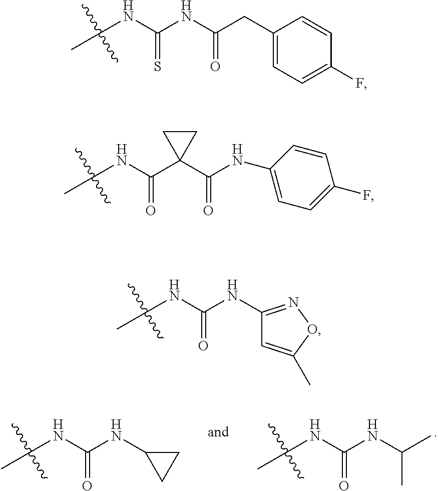

In some embodiments of the first aspect, the compounds have the Formula (I), wherein G is

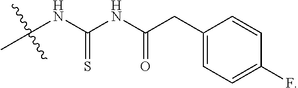 [SMILES: *NC(=S)NC(=O)Cc1ccc(F)cc1]

In some embodiments of the first aspect, the compounds have the Formula (I), wherein G is

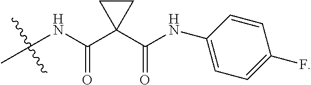 [SMILES: *NC(=O)C1(C(=O)Nc2ccc(F)cc2)CC1]

In some embodiments of the first aspect, the compounds have the Formula (I), wherein G is

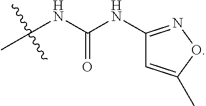 [SMILES: *NC(=O)Nc1cc(C)on1]

In some embodiments of the first aspect, the compounds have the Formula (I), wherein G is

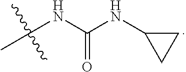 [SMILES: *NC(=O)NC1CC1]

In some embodiments of the first aspect, the compounds have the Formula (I), wherein G is

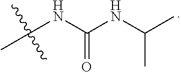 [SMILES: *NC(=O)NC(C)C]

In some embodiments of the first aspect, the compounds have the Formula (I), wherein Q is selected from the group consisting of

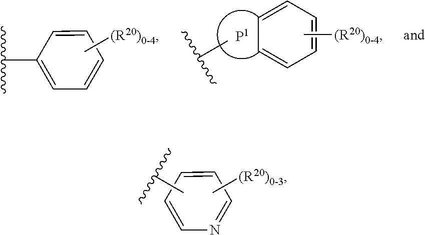

wherein P¹ is a five- to seven-membered ring, including the two shared carbon atoms of the aromatic ring to which P¹ is fused, and wherein P¹ optionally contains between one and three heteroatoms.

In some embodiments of the first aspect, the compounds have the Formula (I), wherein Q is selected from the group consisting of phenyl, napthyl, 1,2,3,4-tetrahydronaphthyl, indanyl, benzodioxanyl, benzofuranyl, phenazinyl, phenothiazinyl, phenoxazinyl, tetrahydroisoquinolyl, pyrrolyl, pyrazolyl, pyrazolidinyl, imidazolyl, imidazolinyl, imidazolidinyl, tetrahydropyridinyl, pyridinyl, pyrazinyl, pyrimidinyl, pyridazinyl, oxazolyl, oxazolinyl, oxazolidinyl, triazolyl, isoxazolyl, isoxazolidinyl, thiazolyl, thiazolinyl, thiazolidinyl, isothiazolyl, isothiazolidinyl, indolyl, isoindolyl, indolinyl, isoindolinyl, octahydroindolyl, octahydroisoindolyl, quinolyl, isoquinolyl, benzimidazolyl, thiadiazolyl, benzopyranyl, benzothiazolyl, benzoxazolyl, furyl, thienyl, benzothieliyl, and oxadiazolyl; each optionally substituted with between one and four of R²⁰.

In some embodiments of the first aspect, the compounds have the Formula (I), wherein Q is phenyl or C₃cycloalkyl.

In some embodiments of the first aspect, the compounds have the Formula (I), wherein Q is phenyl substituted with one or two independently selected R²⁰.

In some embodiments of the first aspect, the compounds have the Formula (I), wherein Q is phenyl substituted with one R²⁰, wherein the R²⁰ is selected from the group consisting of —P(O)(Me)₂, —CH₃, F, —CF₃, —C(O)—NH₂, —S(O)₂CH₃, Cl, —OCF₃, —OMe, Br, —S(O)₂—NH₂, —COOCH₃, —C(O)NH(CH₃) and —C(O)N(CH₃)(CH₃).

In some embodiments of the first aspect, the compounds have the Formula (I), wherein Q is C₃cycloalkyl.

In some embodiments of the first aspect, the compounds are selected from the group consisting of

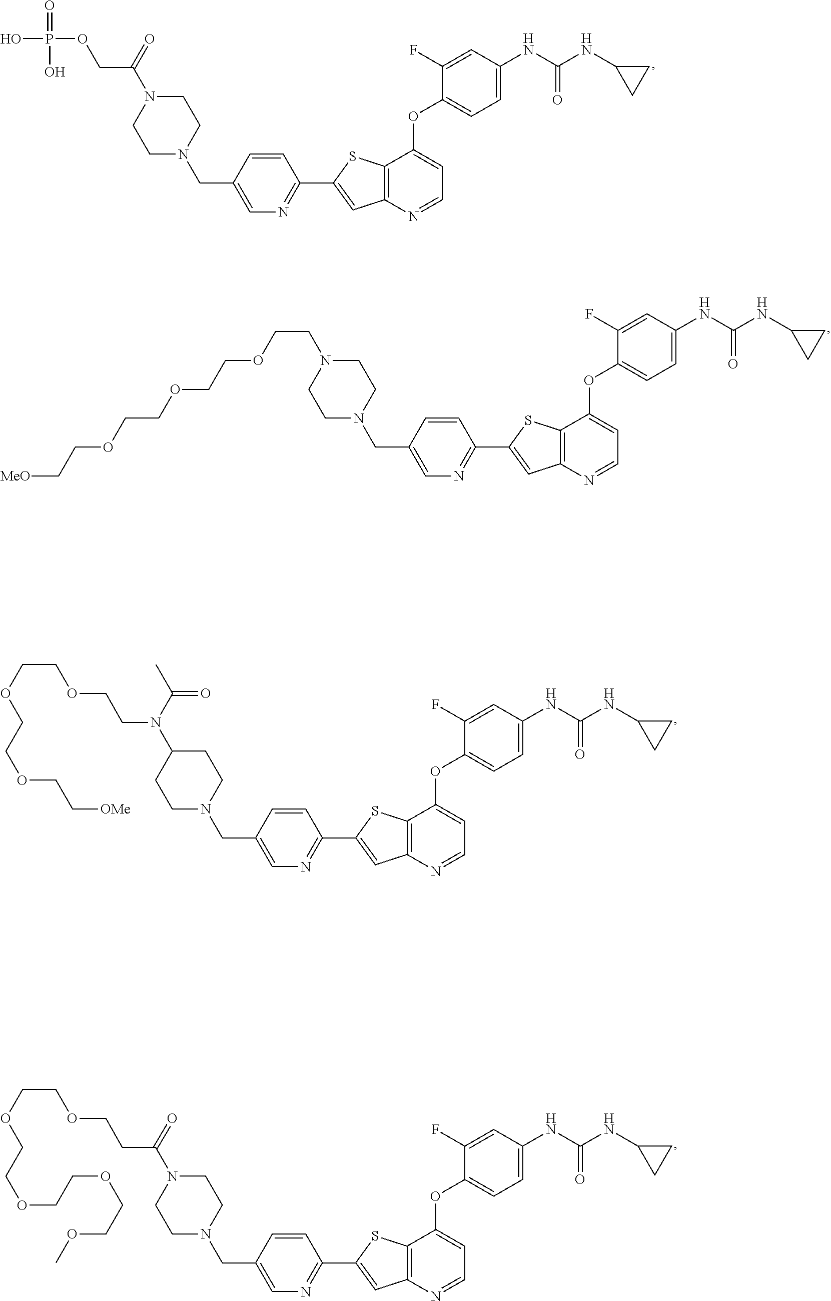 [SMILES: O=C(Nc1ccc(Oc2ccnc3cc(-c4ccc(CN5CCN(C(=O)COP(=O)(O)O)CC5)cn4)sc23)c(F)c1)NC1CC1 ; COCCOCCOCCOCCN1CCN(Cc2ccc(-c3cc4nccc(Oc5ccc(NC(=O)NC6CC6)cc5F)c4s3)nc2)CC1 ; COCCOCCOCCOCCN(C(C)=O)C1CCN(Cc2ccc(-c3cc4nccc(Oc5ccc(NC(=O)NC6CC6)cc5F)c4s3)nc2)CC1 ; COCCOCCOCCOCCOCCC(=O)N1CCN(Cc2ccc(-c3cc4nccc(Oc5ccc(NC(=O)NC6CC6)cc5F)c4s3)nc2)CC1]

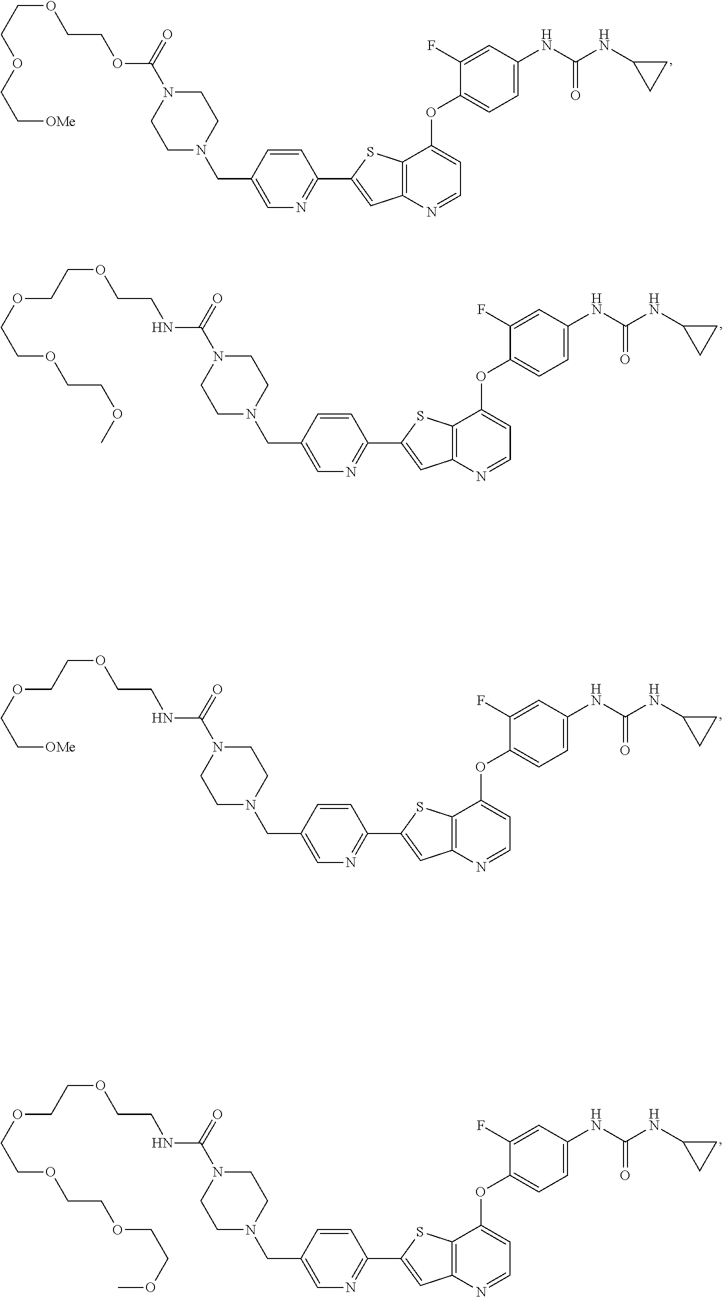 [SMILES: COCCOCCOCCOC(=O)N1CCN(Cc2ccc(-c3cc4nccc(Oc5ccc(NC(=O)NC6CC6)cc5F)c4s3)nc2)CC1 ; COCCOCCOCCOCCNC(=O)N1CCN(Cc2ccc(-c3cc4nccc(Oc5ccc(NC(=O)NC6CC6)cc5F)c4s3)nc2)CC1 ; COCCOCCOCCNC(=O)N1CCN(Cc2ccc(-c3cc4nccc(Oc5ccc(NC(=O)NC6CC6)cc5F)c4s3)nc2)CC1 ; COCCOCCOCCOCCOCCNC(=O)N1CCN(Cc2ccc(-c3cc4nccc(Oc5ccc(NC(=O)NC6CC6)cc5F)c4s3)nc2)CC1]

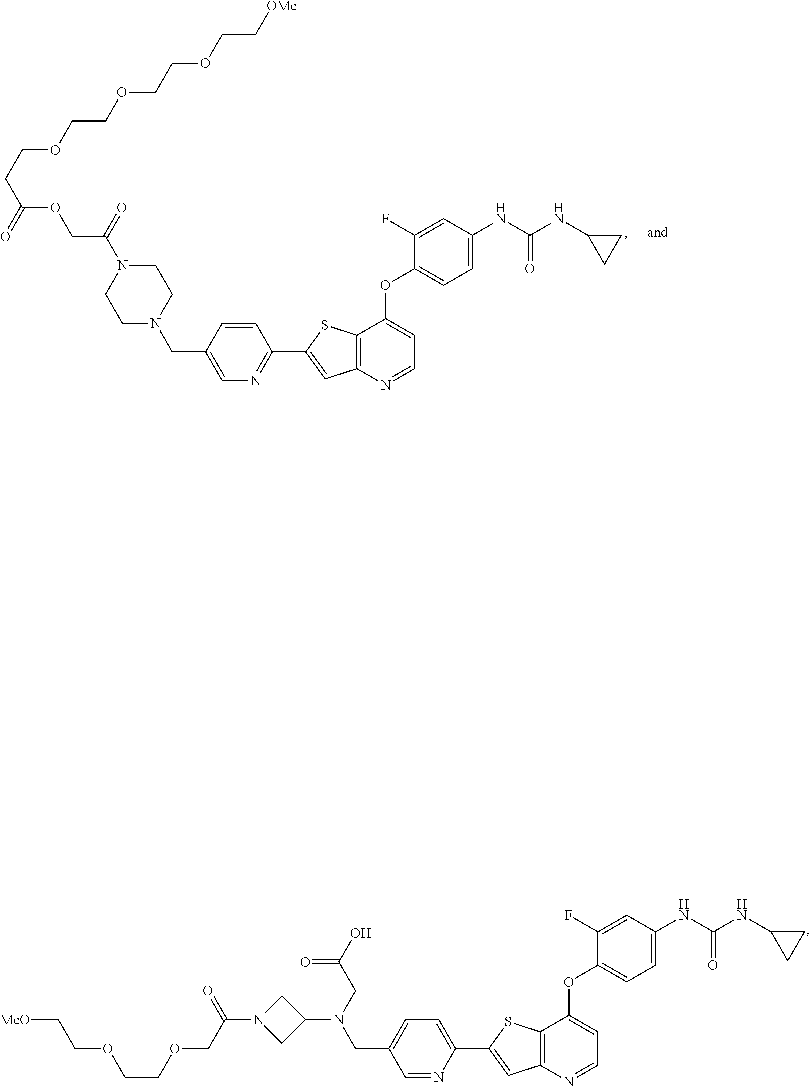 [SMILES: COCCOCCOCCOCCC(=O)OCC(=O)N1CCN(Cc2ccc(-c3cc4nccc(Oc5ccc(NC(=O)NC6CC6)cc5F)c4s3)nc2)CC1 ; COCCOCCOCC(=O)N1CC(N(CC(=O)O)Cc2ccc(-c3cc4nccc(Oc5ccc(NC(=O)NC6CC6)cc5F)c4s3)nc2)C1]

including N-oxides, hydrates, solvates, tautomers, pharmaceutically acceptable salts, prodrugs and complexes thereof, and racemic and scalemic mixtures, diastereomers and enantiomers thereof.

In one embodiment of the first aspect, the compound is

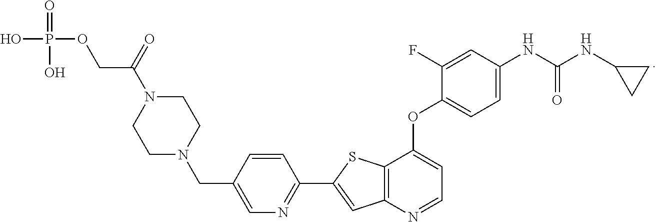 [SMILES: O=C(Nc1ccc(Oc2ccnc3cc(-c4ccc(CN5CCN(C(=O)COP(=O)(O)O)CC5)cn4)sc23)c(F)c1)NC1CC1]

In one embodiment of the first aspect, the compound is

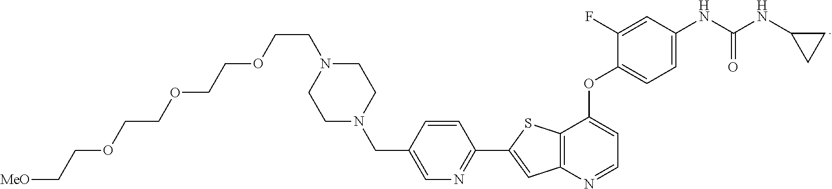 [SMILES: COCCOCCOCCOCCN1CCN(Cc2ccc(-c3cc4nccc(Oc5ccc(NC(=O)NC6CC6)cc5F)c4s3)nc2)CC1]

In one embodiment of the first aspect, the compound is

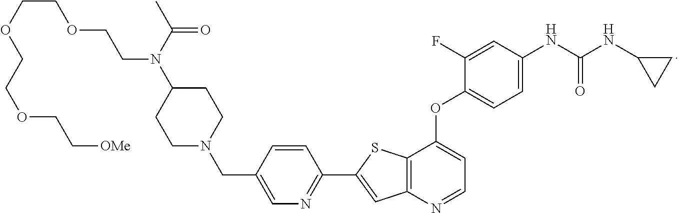 [SMILES: COCCOCCOCCOCCN(C(C)=O)C1CCN(Cc2ccc(-c3cc4nccc(Oc5ccc(NC(=O)NC6CC6)cc5F)c4s3)nc2)CC1]

In one embodiment of the first aspect, the compound is

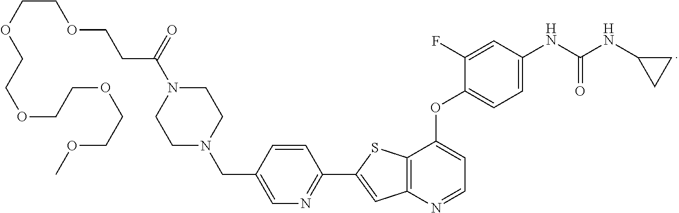 [SMILES: COCCOCCOCCOCCOCCC(=O)N1CCN(Cc2ccc(-c3cc4nccc(Oc5ccc(NC(=O)NC6CC6)cc5F)c4s3)nc2)CC1]

In one embodiment of the first aspect, the compound is

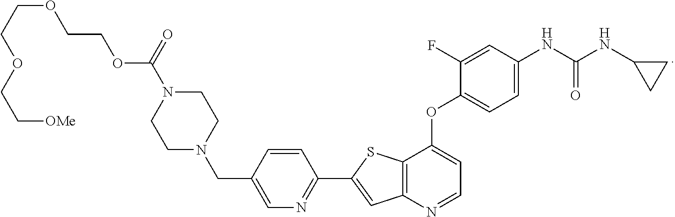 [SMILES: COCCOCCOCCOC(=O)N1CCN(Cc2ccc(-c3cc4nccc(Oc5ccc(NC(=O)NC6CC6)cc5F)c4s3)nc2)CC1]

In one embodiment of the first aspect, the compound is

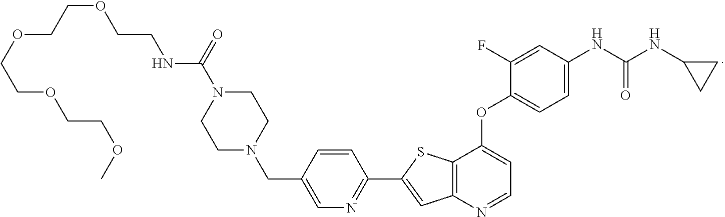 [SMILES: COCCOCCOCCOCCNC(=O)N1CCN(Cc2ccc(-c3cc4nccc(Oc5ccc(NC(=O)NC6CC6)cc5F)c4s3)nc2)CC1]

In one embodiment of the first aspect, the compound is

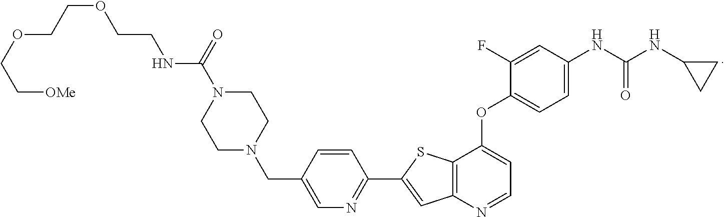 [SMILES: COCCOCCOCCNC(=O)N1CCN(Cc2ccc(-c3cc4nccc(Oc5ccc(NC(=O)NC6CC6)cc5F)c4s3)nc2)CC1]

In one embodiment of the first aspect, the compound is

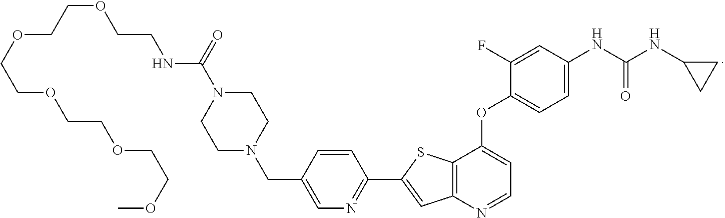 [SMILES: COCCOCCOCCOCCOCCNC(=O)N1CCN(Cc2ccc(-c3cc4nccc(Oc5ccc(NC(=O)NC6CC6)cc5F)c4s3)nc2)CC1]

In one embodiment of the first aspect, the compound is

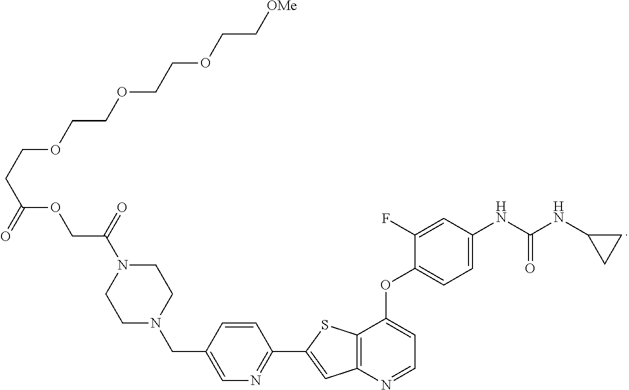 [SMILES: COCCOCCOCCOCCC(=O)OCC(=O)N1CCN(Cc2ccc(-c3cc4nccc(Oc5ccc(NC(=O)NC6CC6)cc5F)c4s3)nc2)CC1]

In one embodiment of the first aspect, the compound is

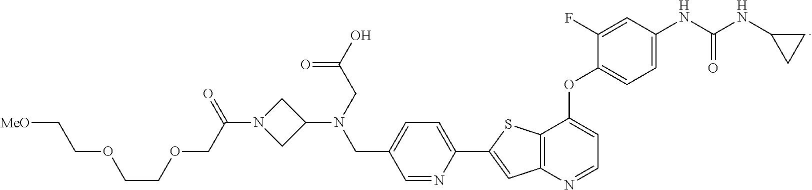 [SMILES: COCCOCCOCC(=O)N1CC(N(CC(=O)O)Cc2ccc(-c3cc4nccc(Oc5ccc(NC(=O)NC6CC6)cc5F)c4s3)nc2)C1]

Compounds of Formula I may generally be prepared according to the following Schemes. Tautomers and solvates (e.g., hydrates) of the compounds of above formulas are also within the scope of the present invention. Methods of solvation are generally known in the art. Accordingly, the compounds of the present invention may be in the free, hydrate or salt form, and may be obtained by methods exemplified by the following schemes below.

The following examples and preparations describe the manner and process of making and using the invention and are illustrative rather than limiting. It should be understood that there may be other embodiments which fall within the spirit and scope of the invention as defined by the claims appended hereto.

Compounds according to the invention include but are not limited to those described in the examples below. Compounds were named using Chemdraw Ultra (versions 10.0, 10.0.4 or version 8.0.3), which are available through Cambridgesoft (www.Cambridgesoft.com, 100 Cambridge Park Drive, Cambridge, Mass. 02140, or were derived therefrom.

The data presented herein demonstrate the inhibitory effects of the kinase inhibitors of the invention. These data lead one to reasonably expect that the compounds of the invention are useful not only for inhibition of kinase activity, protein tyrosine kinase activity, or embodiments thereof, such as, VEGF receptor signaling, but also as therapeutic agents for the treatment of proliferative diseases, including cancer and tumor growth and ophthalmic diseases, disorders and conditions.

### Synthetic Schemes and Experimental Procedures

The compounds of the invention can be prepared according to the reaction schemes or the examples illustrated below utilizing methods known to one of ordinary skill in the art. These schemes serve to exemplify some procedures that can be used to make the compounds of the invention. One skilled in the art will recognize that other general synthetic procedures may be used. The compounds of the invention can be prepared from starting components that are commercially available. Any kind of substitutions can be made to the starting components to obtain the compounds of the invention according to procedures that are well known to those skilled in the art.

All reagents and solvents were obtained from commercial sources and used as received. ¹H-NMR spectra were recorded on Mercury Plus Varian 400 MHz instrument in the solvents indicated. Low resolution mass-spectra (LRMS) were acquired on Agilent MSD instrument. Analytical HPLC was performed on Agilent 1100 instrument using Zorbax 3 μm, XDB-C8, 2.1×50 mm column; eluting with methanol/water containing 0.1% formic acid, with a gradient 5-95% methanol in 15 minutes. Automated column chromatography was performed on Biotage SP1 or Biotage SP4 instruments using Biotage® SNAP, SiliaSep™ or SiliaFlash® cartridges. Flash column chromatography was performed using silica gel (SiliaFlash F60, 40-63 μM, pore size 60 Å, SiliCycle®). Preparative column chromatography was performed on Gilson 215 instrument using Phenomenex Luna 15 μm, C18(2) 100A, 250×21 mm column eluting with a mixture methanol/water containing 0.05% of formic acid, with a gradient 0-95% methanol in up to 60 minutes.

### PARTICULAR EXAMPLES

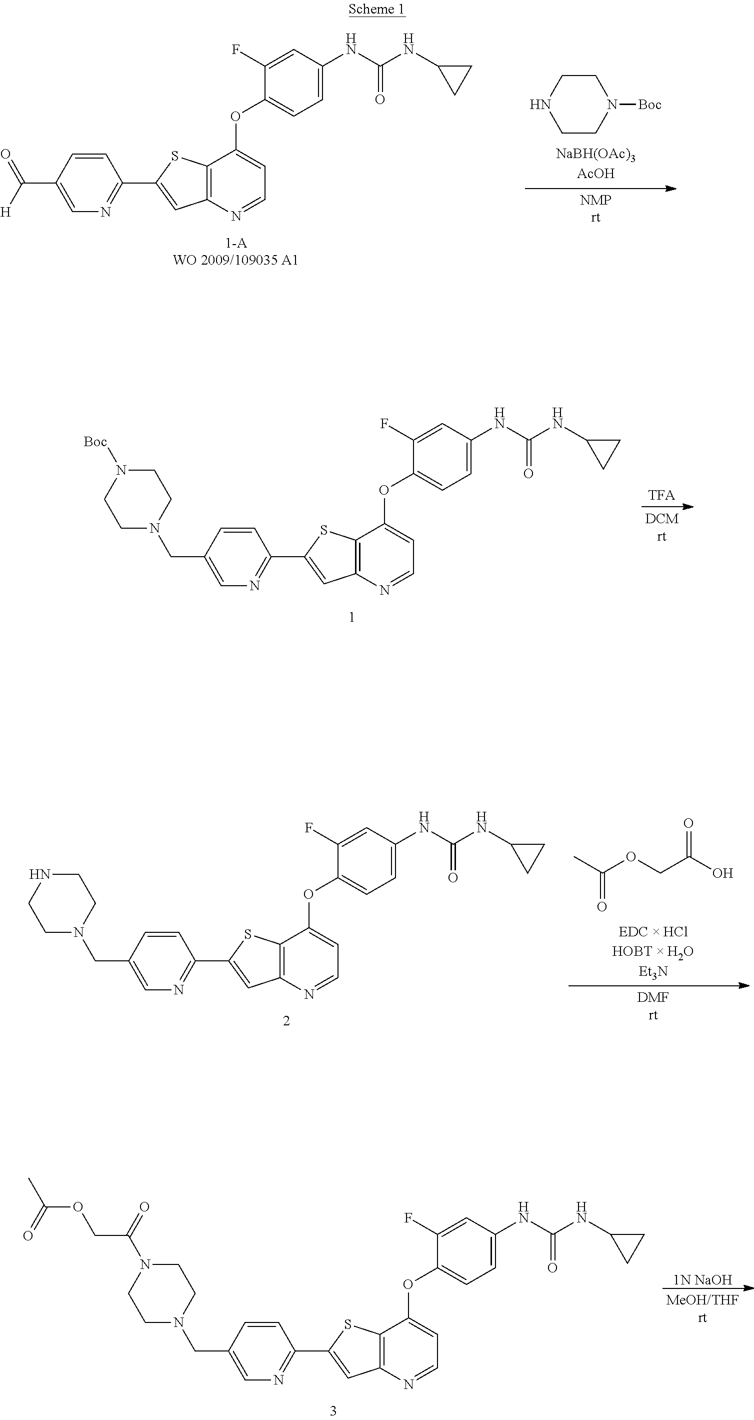

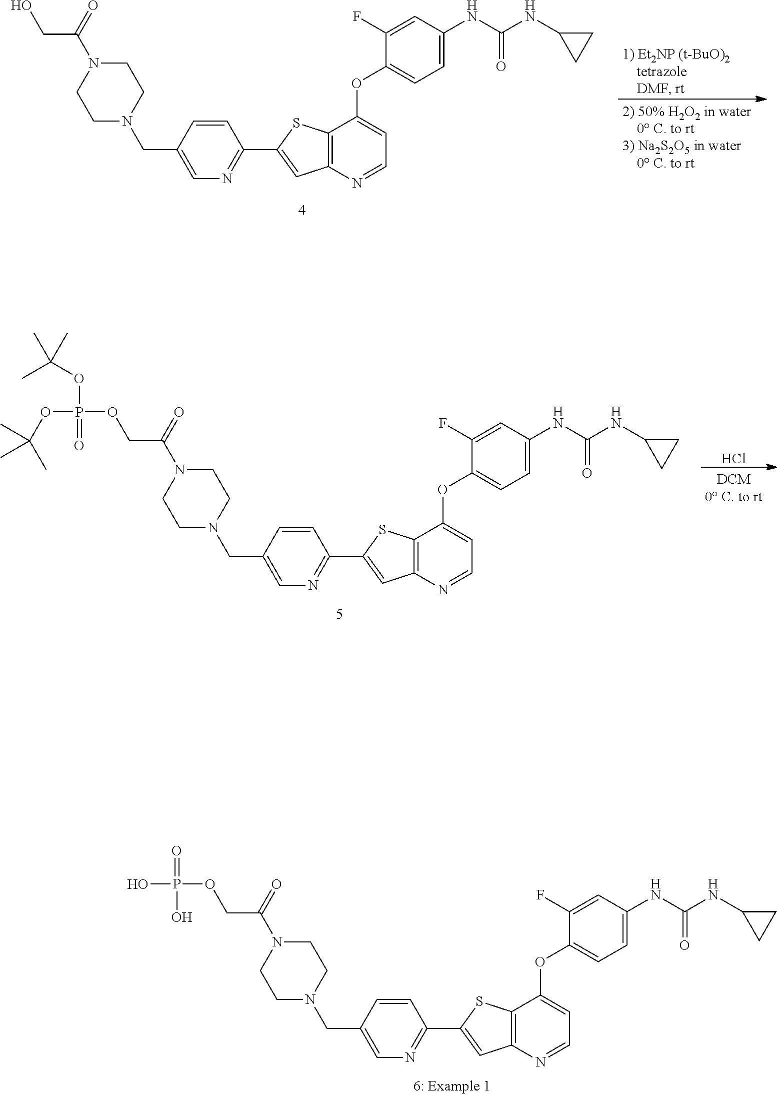

### Example 1

### 2-(4-((6-(7-(4-(3-Cyclopropylureido)-2-fluorophenoxy)thieno[3,2-b]pyridin-2-yl)pyridin-3-yl)methyl)piperazin-1-yl)-2-oxoethyl dihydrogen phosphate (6)

### Step 1. tert-Butyl 4-((6-(7-(4-(3-cyclopropylureido)-2-fluorophenoxy)thieno[3,2-b]-pyridin-2-yl)pyridin-3-yl)methyl)piperazine-1-carboxylate (1)

To a solution of 1-cyclopropyl-3-(3-fluoro-4-(2-(5-formylpyridin-2-yl)thieno[3,2-b]pyridin-7-yloxy)phenyl)urea (1-A) (3.00 g, 6.69 mmol, WO 2009/109035 A1), 1-Bocpiperazine (1.495 g, 8.03 mmol) in NMP (40 ml) at rt under nitrogen were added acetic acid (765 μl, 13.38 mmol) and 15 min later, NaBH(OAc)₃ (4.48 g, 20.07 mmol) portionwise over 2 h. The reaction mixture was stirred at rt overnight, poured into a saturated aqueous solution of sodium bicarbonate, and stirred for 1 h. The precipitated solid was collected by filtration, rinsed with water and dried. The crude product was purified by Biotage (Snap 100 g cartridge; MeOH/DCM: 1/99 to 10/90 over 20 CV), to afford the desired product 1 (3.27 g, 5.29 mmol, 79% yield) as a beige-brown sticky solid (Slightly contaminated by TLC). ¹H NMR (400 MHz, DMSO-d₆) δ (ppm): 8.71 (s, 1H), 8.56 (bd, J=2.0 Hz, 1H), 8.52 (d, J=5.5 Hz, 1H), 8.33 (s, 1H), 8.25 (d, J=8.2 Hz, 1H), 7.87 (dd, J=8.1, 2.1 Hz, 1H), 7.73 (dd, J=13.6, 2.4 Hz, 1H), 7.38 (t, J=9.1 Hz, 1H), 7.20 (bdd, J=8.8, 1.2 Hz, 1H), 6.65 (d, J=5.3 Hz, 1H), 6.57 (bd, J=2.5 Hz, 1H), 3.57 (s, 2H), 4H are hidden by water peak, 2.59-2.51 (m, 1H), 2.42-2.27 (m, 4H), 1.39 (s, 9H), 0.72-0.58 (m, 2H), 0.50-0.36 (m, 2H). MS (m/z): 619.4 (M+H).

### Step 2. 1-Cyclopropyl-3-(3-fluoro-4-(2-(5-(piperazin-1-ylmethyl)pyridin-2-yl)thieno[3,2-b]-pyridin-7-yloxy)phenyl)urea (2)

A solution of compound 1 (3.27 g, 5.29 mmol) and TFA (12.86 ml) in DCM (50 ml) was stirred at rt for 3 h. The reaction mixture was concentrated, diluted with water, stirred for 10 min and poured slowly into a saturated aqueous solution of sodium bicarbonate; the pH was adjusted to around 9-10 with 1N NaOH. The resultant suspension was stirred for 1 h, collected by filtration, rinsed with water, and air-dried. The material was purified by Biotage (Snap 50 g cartridge; 2% of ammonium hydroxide in MeOH/DCM: 05/95 to 30/70 over 20 CV), to afford the desired product 2 (2.097 g, 3.96 mmol, 75% yield, contaminated by 0.1 equiv. of TFA based on the ¹⁹F-NMR spectrum) as a pink sticky powder. The product was used in the next step without any further purification. ¹H NMR (400 MHz, DMSO-d₆) δ (ppm): 8.76 (bs, 1H), 8.54 (d, J=1.4 Hz, 1H), 8.52 (d, J=5.5 Hz, 1H), 8.32 (s, 1H), 8.24 (d, J=8.2 Hz, 1H), 7.85 (dd, J=8.1, 2.1 Hz, 1H), 7.73 (dd, J=13.5, 2.3 Hz, 1H), 7.38 (t, J=9.1 Hz, 1H), 7.20 (bd, J=10.2 Hz, 1H), 6.64 (d, J=5.5 Hz, 1H), 6.62 (bs, 1H), 3.58-3.48 (m, 2H), 2.73-2.64 (m, 4H), 2.59-2.52 (m, 1H), 2.38-2.25 (m, 4H), 0.69-0.62 (m, 2H), 0.46-0.40 (m, 2H), one NH is missing. MS (m/z): 519.6 (M+H).

### Step 3. 2-(4-((6-(7-(4-(3-Cyclopropylureido)-2-fluorophenoxy)thieno[3,2-b]pyridin-2-yl)pyridin-3-yl)methyl)piperazin-1-yl)-2-oxoethyl acetate (3)

To a stirred solution of compound 2 (600 mg, 1.16 mmol), 2-acetoxyacetic acid (205 mg, 1.74 mmol) and tiethylamine (481 μl, 3.47 mmol) in DMF (15 ml) under nitrogen were added HOBT monohydrate (195 mg, 1.27 mmol) and EDC hydrochloride (444 mg, 2.31 mmol). The reaction mixture was stirred at rt overnight, quenched by addition of water, and diluted with AcOEt with traces of MeOH to form a biphasic system. The phases were separated; the organic layer was successively washed with a saturated aqueous solution of sodium bicarbonate and brine, dried over anhydrous magnesium sulfate, filtered and concentrated. The residue was purified by Biotage (Snap 50 g cartridge; MeOH/DCM: 0/100 to 10/90 over 20 CV then 10/90 over 5 CV), to afford the desired product 3 (537 mg, 0.868 mmol, 75% yield) as an off-white sticky solid. MS (m/z): 619.7 (M+H).

### Step 4. 1-Cyclopropyl-3-(3-fluoro-4-(2-(5-(4-(2-hydroxyacetyl)piperazin-1-yl)methyl)pyridin-2-yl)thieno[3,2-b]pyridin-7-yloxy)phenyl)urea (4)

To a stirred solution of compound 3 (0.94 g, 1.52 mmol) in a mixture of MeOH/THF (30 ml/25 ml) was added 1N NaOH (3.8 ml, 3.80 mmol). The reaction mixture was stirred at rt for 3 h, concentrated, diluted in a minimum of methanol in water, neutralyzed with a saturated aqueous solution of ammonium chloride (pH around 8). The solid was collected by filtration, rinsed with water and dried to afford the desired product 4 (826 mg, 1.43 mmol, 94% yield) as an off-white fluffy solid. ¹H NMR (400 MHz, DMSO-d₆) δ (ppm): 8.77-8.69 (m, 1H), 8.57 (d, J=1.6 Hz, 1H), 8.52 (d, J=5.5 Hz, 1H), 8.34 (s, 1H), 8.26 (d, J=8.0 Hz, 1H), 7.88 (dd, J=8.1, 2.1 Hz, 1H), 7.73 (dd, J=13.5, 2.3 Hz, 1H), 7.38 (t, J=9.1 Hz, 1H), 7.20 (bd, J=9.2 Hz, 1H), 6.65 (d, J=4.9 Hz, 1H), 6.63-6.56 (m, 1H), 4.55 (t, J=5.5 Hz, 1H), 4.07 (d, J=5.5 Hz, 2H), 3.60 (s, 2H), 3.53-3.43 (m, 2H), 2H are hidden, 2.59-2.51 (m, 1H), 2.45-2.33 (m, 4H), 0.72-0.58 (m, 2H), 0.50-0.36 (m, 2H). MS (m/z): 577.5 (M+H).

### Step 5. Di-tert-butyl 2-(4-((6-(7-(4-(3-cyclopropylureido)-2-fluorophenoxy)thieno[3,2-b]pyridin-2-yl)pyridin-3-yl)methyl)piperazin-1-yl)-2-oxoethyl phosphate (5)

To a stirred solution of compound 4 (150 mg, 0.26 mmol) and tetrazole (109 mg, 1.56 mmol) in DMF (10 ml) under nitrogen was added (t-BuO)₂PNEt₂ (724 μl, 2.60 mmol) in five portions over 4.5 hrs. The reaction mixture was stirred at rt overnight, cooled to 0° C. and a solution of hydrogen peroxide (319 μl, 5.20 mmol, 50% in water) was slowly added. The reaction mixture was allowed to warm to rt over 10 min, then stirred rt for 1 h. The reaction mixture was cooled again to 0° C. and an aqueous solution of sodium metabisulfite (1.53 g in 10 ml of water) was slowly added. The reaction mixture was allowed to warm to rt over 15 min, and quenched with a saturated aqueous solution of sodium bicarbonate to form a precipitate. After stirring for 30 min, the solid was collected by filtration, rinsed with water and air-dried. The crude material was purified by Biotage (Snap 25 g cartridge; MeOH/DCM: 1/99 to 15/85 over 30 CV), to afford the desired product 5 (78 mg, 0.1 mmol, 39% yield) as a colorless sticky film. It was used directly in the next step without any further purification. MS (m/z): 769.6 (M+H).

### Step 6. 2-(4-((6-(7-(4-(3-Cyclopropylureido)-2-fluorophenoxy)thieno[3,2-b]pyridin-2-yl)pyridin-3-yl)methyl)piperazin-1-yl)-2-oxoethyl dihydrogen phosphate (6)

To a stirred solution of compound 5 (78 mg, 0.1 mmol) in DCM (20 ml) at 0° C. was added a solution of 4M HCl in 1,4-dioxane (254 μl, 1.02 mmol). The reaction mixture was stirred for 40 min, and concentrated by co-evaporation with methanol. The residue was purified twice by Biotage [Snap 30 g cartridge KP-C18-HS (reverse phase); MeOH/water (Millipore): 20/80 to 95/05 over 40 CV (Snap 50 g cartridge, 40 ml/min)], to afford the desired product 6 (17 mg, 0.026 mmol, 25% yield) as an off-white fluffy solid. ¹H NMR (400 MHz, DMSO-d₆) δ (ppm): 2OH phosphate are missing, 8.89 (s, 1H), 8.56 (bs, 1H), 8.51 (d, J=5.3 Hz, 1H), 8.32 (s, 1H), 8.24 (d, J=8.0 Hz, 1H), 7.87 (dd, J=8.0, 1.8 Hz, 1H), 7.73 (dd, J=13.7, 2.3 Hz, 1H), 7.37 (t, J=9.1 Hz, 1H), 7.22 (d, J=8.6 Hz, 1H), 6.71 (bs, 1H), 6.63 (d, J=5.1 Hz, 1H), 4.44-4.32 (m, 2H), 3.59 (s, 2H), 4H are hidden by water's peak, 2.60-2.52 (m, 1H), 2.48-2.32 (m, 4H), 0.71-0.57 (m, 2H), 0.49-0.36 (m, 2H). MS (m/z): 657.5 (M+H).

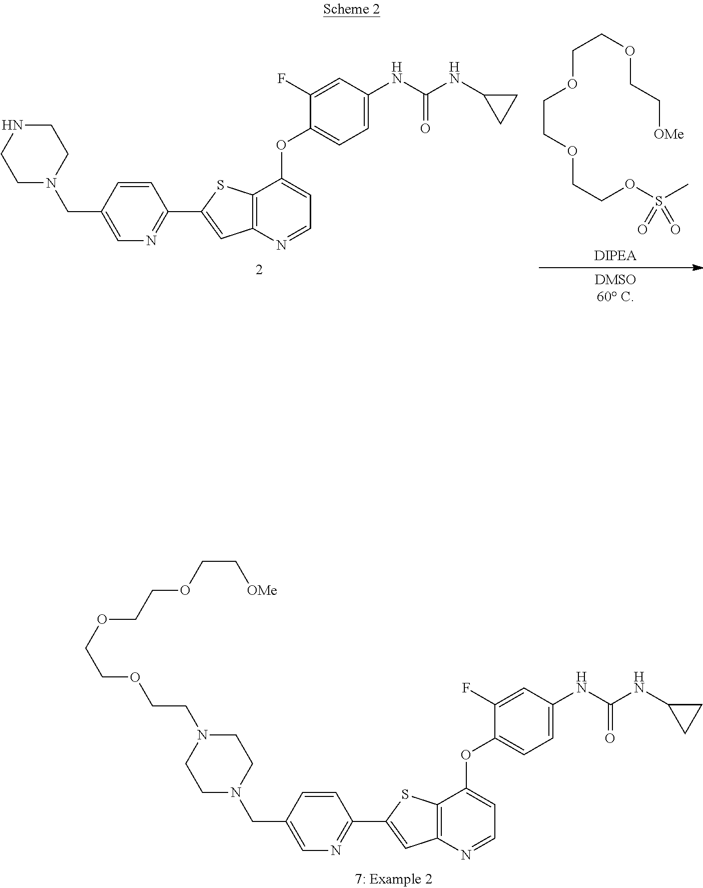

### Example 2

### 1-(4-(2-(5-((4-(2,5,8,11-Tetraoxamidecan-13-yl)piperazin-1-yl)methyl)pyridin-2-yl)thieno[3,2-b]pyridin-7-yloxy)-3-fluorophenyl)-3-cyclopropylurea (7)

To a stirred solution of compound 2 (400 mg, 0.77 mmol) and DIPEA (168 μl, 0.96 mmol) in DMSO (5 ml) under nitrogen at rt was added 2,5,8,11-tetraoxamidecan-13-yl methanesulfonate (133 mg, 0.46 mmol, K Fukase, et. al. SynLett., 2005, 2342-2346), and the reaction mixture was heated at 60° C. for 2 h. More 2,5,8,11-tetraoxamidecan-13-yl methanesulfonate (450 mg, 1.58 mmol) was added, and the reaction mixture was heated at 60° C. overnight. The reaction mixture was diluted with AcOEt, and successively washed with water, a saturated aqueous solution of sodium bicarbonate, water and brine, dried over anhydrous magnesium sulfate, filtered and concentrated. The residue was purified twice by Biotage (Snap 25 g cartridge; 2% of ammonium hydroxide in MeOH/DCM: 1/99 to 10/90 over 20 CV, then 10/90 to 20/80 over 10 CV; Snap 10 g cartridge; 2% of ammonium hydroxide in MeOH/DCM: 1/99 to 20/80 over 30 CV), to afford the desired product 7 (49 mg, 0.07 mmol, 17% yield) as a pale yellow sticky oil. ¹H NMR (400 MHz, DMSO-d₆) δ (ppm): 8.71 (s, 1H), 8.54 (bs, 1H), 8.52 (d, J=5.3 Hz, 1H), 8.32 (s, 1H), 8.24 (d, J=8.2 Hz, 1H), 7.85 (dd, J=7.8, 1.8 Hz, 1H), 7.73 (dd, J=13.5, 2.3 Hz, 1H), 7.38 (t, J=9.1 Hz, 1H), 7.20 (bd, J=8.4 Hz, 1H), 6.64 (d, J=4.9 Hz, 1H), 6.57 (bd, J=2.0 Hz, 1H), 3.54 (s, 2H), 3.52-3.46 (m, 12H), 3.43-3.37 (m, 2H), 3.22 (s, 3H), 2.59-2.34 (m, 11H), 0.73-0.58 (m, 2H), 0.50-0.36 (m, 2H). MS (m/z): 709.7 (M+H).

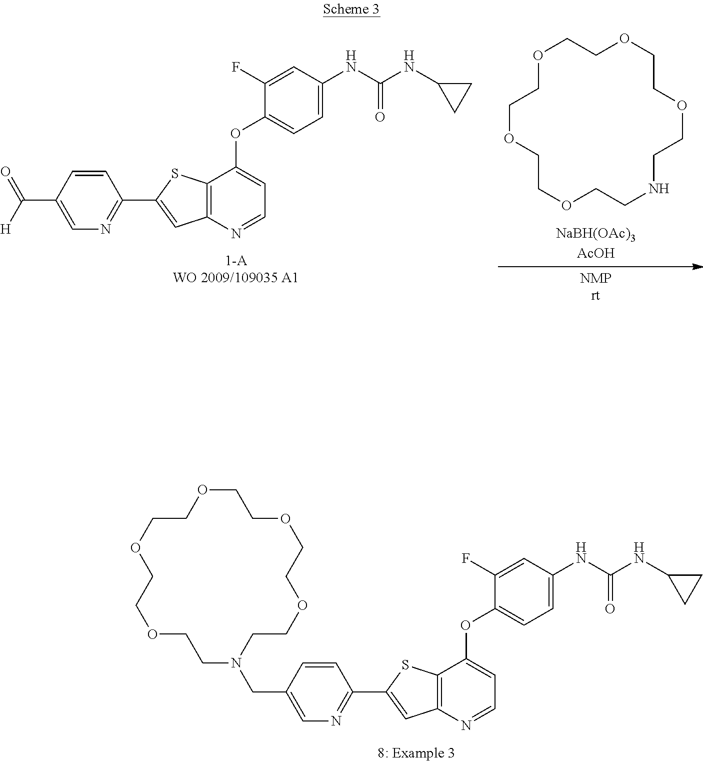

### Example 3

### 1-(4-(2-(5-((1,4,7,10,13-Pentaoxa-16-azacyclooctadecan-16-yl)methyl)pyridin-2-yl)thieno[3,2-b]pyridin-7-yloxy)-3-fluorophenyl)-3-cyclopropylurea (8)

To a solution of 1-A (300 mg, 0.67 mmol, scheme 1), 1-aza-18-crown-6 ether (352 mg, 1.338 mmol) in NMP (10 ml) and acetic acid (77 μl, 1.34 mmol) at rt under nitrogen was added NaBH(OAc)₃ (448 mg, 2.01 mmol). The reaction mixture was stirred at rt for 2 h, quenched by addition of water, stirred for 30 min and slowly neutralyzed with 1N NaOH (pH around 11). The resultant suspension was stirred and sonicated for 10 min, and the solid was collected by filtration, rinsed with water and air-dried. The mother liquor was extracted with AcOEt. The extract was washed with water, and concentrated. The residue was combined with the collected solid and purified by Biotage [Snap 30 g cartridge KP-C18-HS (reverse phase); MeOH/water (Millipore): 20/80 to 95/05 over 40 CV (size choice: Snap 50 g cartridge, 40 ml/min)], to afford the desired product 8 (196 mg, 0.28 mmol, 42% yield) as a pale brown sticky solid. ¹H NMR (500 MHz, DMSO-d₆) δ (ppm): 8.74 (s, 1H), 8.57 (bd, J=1.5 Hz, 1H), 8.51 (d, J=5.4 Hz, 1H), 8.31 (s, 1H), 8.21 (d, J=8.1 Hz, 1H), 7.89 (dd, J=8.1, 1.9 Hz, 1H), 7.73 (dd, J=13.5, 2.4 Hz, 1H), 7.37 (t, J=9.1 Hz, 1H), 7.19 (d, J=8.6 Hz, 1H), 6.63 (d, J=5.4 Hz, 1H), 6.60 (bs, 1H), 3.71 (s, 2H), 3.63-3.43 (m, 20H), 2.68 (t, J=5.7 Hz, 4H), 2.58-2.51 (m, 1H), 0.71-0.58 (m, 2H), 0.48-0.36 (m, 2H). MS (m/z): 696.6 (M+H).

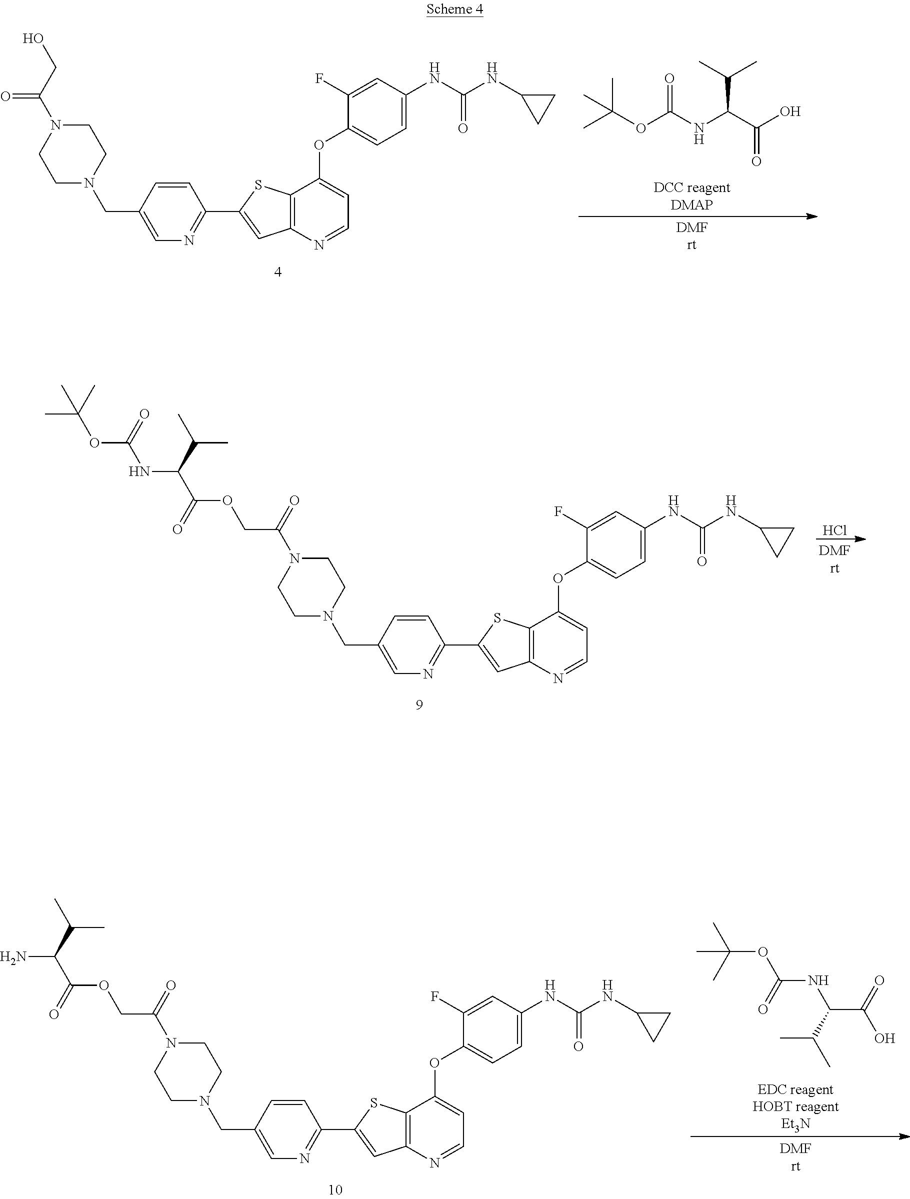

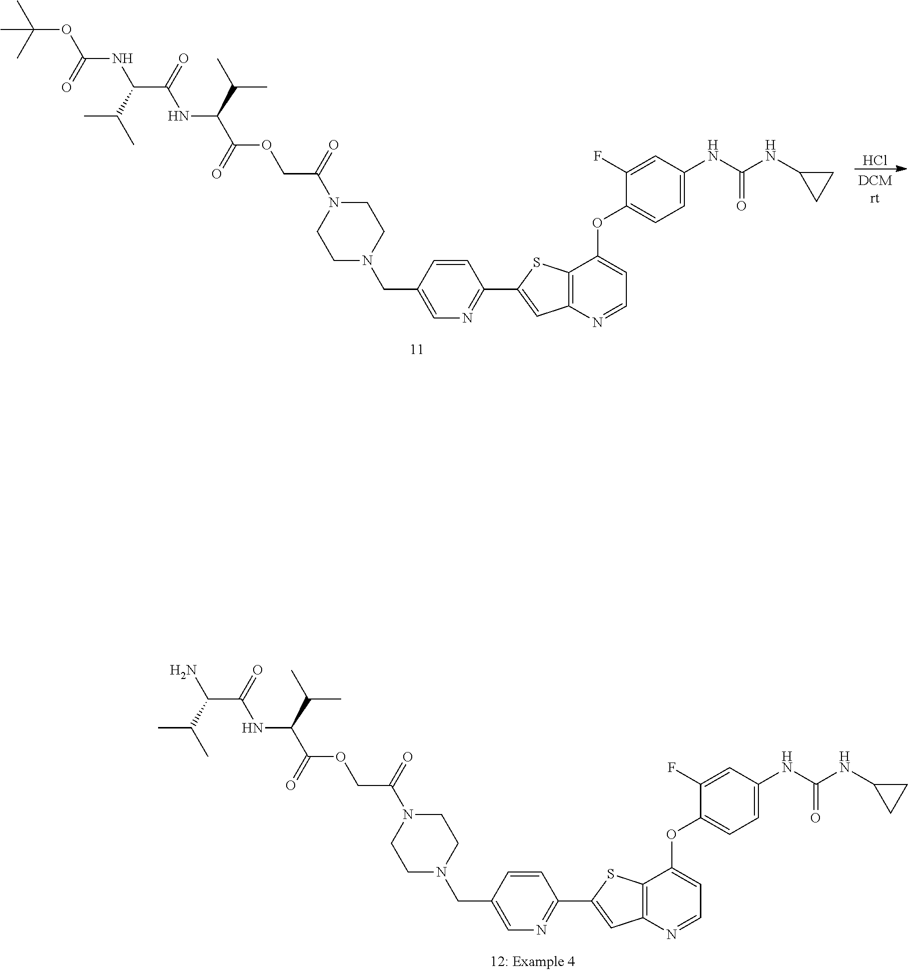 [SMILES: CC(C)[C@H](NC(=O)OC(C)(C)C)C(=O)N[C@H](C(=O)OCC(=O)N1CCN(Cc2ccc(-c3cc4nccc(Oc5ccc(NC(=O)NC6CC6)cc5F)c4s3)nc2)CC1)C(C)C ; Cl ; CC(C)[C@H](N)C(=O)N[C@H](C(=O)OCC(=O)N1CCN(Cc2ccc(-c3cc4nccc(Oc5ccc(NC(=O)NC6CC6)cc5F)c4s3)nc2)CC1)C(C)C]

### Example 4

### (S)-2-(4-(6-(7-(4-(3-Cyclopropylureido)-2-fluorophenoxy)thieno[3,2-b]pyridin-2-yl)pyridin-3-yl)methyl)piperazin-1-yl)-2-oxoethyl 2-((S)-2-amino-3-methylbutanamido-3-methylbutanoate (12)

### Step 1. (S)-2-(4-((6-(7-(4-(3-Cyclopropylureido)-2-fluorophenoxy)thieno[3,2-b]pyridin-2-yl)pyridin-3-yl)methyl)piperazin-1-yl)-2-oxoethyl 2-tert-butoxycarbonylamino-3-methylbutanoate (9)

To a stirred solution of compound 4 (150 mg, 0.26 mmol, scheme 1), Boc-Val-OH (141 mg, 0.65 mmol) and DMAP (32 mg, 0.26 mmol) in DMF (7 ml) under nitrogen was added DCC reagent (215 mg, 1.04 mmol), and the reaction mixture was stirred at rt overnight. The reaction mixture was diluted with AcOEt and successively washed with water, a saturated aqueous solution of sodium bicarbonate, a saturated aqueous solution of ammonium chloride, water and brine, dried over anhydrous magnesium sulfate, filtered, and concentrated. The residue was purified by Biotage (Snap 25 g cartridge; MeOH/DCM: 1/99 to 10/90 over 30 CV), to afford the desired product 9 (203 mg, quantitative yield) as a colorless sticky film. MS (m/z): 776.2 (M+H).

### Step 2. (S)-2-(4-((6-(7-(4-(3-Cyclopropylureido)-2-fluorophenoxy)thieno[3,2-b]pyridin-2-yl)pyridin-3-yl)methyl)piperazin-1-yl-2-oxoethyl) 2-amino-3-methylbutanoate (10)

To a solution of compound 9 (203 mg, 0.26 mmol) in DCM (20 ml) was added a solution of HCl (1.31 ml, 4M in 1,4-dioxane). The reaction mixture was stirred for 1 h, and the resultant precipitate was collected by filtration, rinsed with DCM, air-dried for a few minutes and dissolved in MeOH. The resultant solution was concentrated and the residue was dried under high vacuum to afford the desired product 10 as an yellow sticky foam (presumably a hydrochloride salt with unknown stoichiochemistry). The material was used in the next step without any further purification. MS (m/z): 676.5 (M+H).

### Step 3. (S)-2-(4-((6-(7-(4-(3-Cyclopropylureido)-2-fluorophenoxy)thieno[3,2-b]pyridin-2-yl)pyridin-3-yl)methyl)piperazin-1-yl)-2-oxoethyl 2-((S)-2-(tert-butoxycarbonylamino)-3-methylbutanamido)-3-methylbutanoate (11)

To a stirred solution of compound 10 (0.26 mmol, from the previous experiment), Boc-L-Val-OH (141 mg, 0.65 mmol) and triethylamine (180 μl, 1.30 mmol) in DMF (10 ml) under nitrogen were added HOBT-monohydrate (44 mg, 0.29 mmol) and EDC-hydrochloride (150 mg, 0.78 mmol) reagents, and the reaction mixture was stirred at rt overnight. The reaction mixture was partitioned between AcOEt and a saturated aqueous solution of sodium bicarbonate. After separation, the organic layer was successively washed with a saturated aqueous solution of sodium bicarbonate, a saturated aqueous solution of ammonium chloride, water and brine, dried over anhydrous magnesium sulfate, filtered, and concentrated. The residue was purified by Biotage (SiliaFlash 12 g cartridge; MeOH/DCM: 1/99 to 10/90 over 30 CV), to afford the desired product 11 (160 mg, 0.18 mmol, 70% yield over 2 steps) as a colorless sticky foam. MS (m/z): 875.6 (M+H).

### Step 4. (S)-2-(4-((6-(7-(4-(3-Cyclopropylureido)-2-fluorophenoxy)thieno[3,2-b]pyridin-2-yl)pyridin-3-yl)methyl)piperazin-1-yl)-2-oxoethyl 2-((S)-2-amino-3-methylbutanamido)-3-methylbutanoate (12)

To a solution of compound 11 (160 mg, 0.18 mmol) in DCM (20 ml) was added a solution of HCl (0.91 ml, 4M in 1,4-dioxane). The reaction mixture was stirred for 75 min, concentrated and neutralyzed with 2% solution of ammonium hydroxide in MeOH. The crude product was purified twice by Biotage (Snap 10 g cartridge; 2% of ammonium hydroxyde in MeOH/DCM: 5/95 to 15/85 over 30 CV), to afford the desired product 12 (90 mg, 0.12 mmol, 63% yield) as a white sticky solid. ¹H NMR (400 MHz, DMSO-d₆) δ (ppm): 8.73 (s, 1H), 8.58 (bd, J=1.6 Hz, 1H), 8.52 (d, J=5.3 Hz, 1H), 8.34 (s, 1H), 8.26 (d, J=8.0 Hz, 1H), 8.10 (d, J=8.6 Hz, 1H), 7.88 (dd, J=8.1, 2.1 Hz, 1H), 7.73 (dd, J=13.6, 2.4 Hz, 1H), 7.38 (t, J=9.1 Hz, 1H), 7.20 (dd, J=8.9, 1.3 Hz, 1H), 6.65 (dd, J=5.4, 0.7 Hz, 1H), 6.59 (bd, J=2.3 Hz, 1H), AB system (δ_{A}=4.88, δ_{B}=4.80, J_{AB}=14.8 Hz, 2H), 4.35-4.28 (m, 1H), 3.60 (s, 2H), 3.50-3.34 (m, 4H), 3.04 (d, J=4.9 Hz, 1H), 2.59-2.51 (m, 1H), 2.47-2.32 (m, 4H), 2.20-2.10 (m, 1H), 1.97-1.86 (m, 1H), 1.84-1.68 (m, 2H), 0.93 (d, J=6.8 Hz, 6H), 0.88 (d, J=6.8 Hz, 3H), 0.77 (d, J=6.8 Hz, 3H), 0.72-0.58 (m, 2H), 0.49-0.36 (m, 2H). MS (m/z): 775.7 (M+H).

Compound 13 (example 5) was prepared in two steps by coupling compound 10 with the corresponding protected aminoacid similarly to compound 12 (example 4, scheme 4). Compounds 14-17 (examples 6-9) were prepared in one step by coupling compound 4 with the corresponding protected aminoacid or protected dipeptide similarly to compound 9 (scheme 4).

TABLE 1
Characterization of compounds 13-17 (examples 5-9).

| Cpd | Ex. | Structure | Characterization |
|---|---|---|---|
| 13 | 5 | 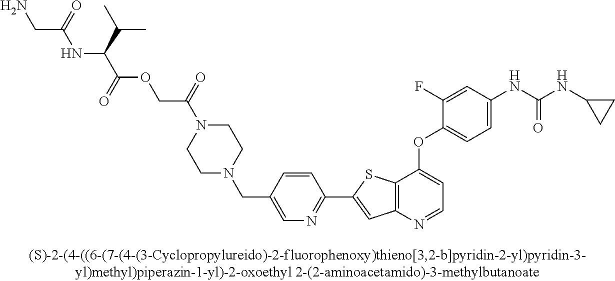 [SMILES: CC(C)[C@H](NC(=O)CN)C(=O)OCC(=O)N1CCN(Cc2ccc(-c3cc4nccc(Oc5ccc(NC(=O)NC6CC6)cc5F)c4s3)nc2)CC1] | ¹H NMR (400 MHz, DMSO-d₆) δ (ppm): 8.75 (s, 1H), 8.58 (bd, J = 1.8 Hz, 1H), 8.52 (d, J = 5.5 Hz, 1H), 8.34 (s, 1H), 8.32 (d, J = 9.0 Hz, 1H), 8.26 (d, J = 8.2 Hz, 1H), 7.88 (dd, J = 8.2, 2.0 Hz, 1H), 7.73 (dd, J = 13.6, 2.4 Hz, 1H), 7.38 (t, J = 9.0 Hz, 1H), 7.20 (bd, J = 10.2 Hz, 1H), 6.65 (bd, J = 5.5 Hz, 1H), 6.60 (bd, J = 2.3 Hz, 1H), AB system (δ_{A} = 4.89, δ_{B} = 4.82, J_{AB} = 14.8 Hz, 2H), 4.41- 4.33 (m, 1H), 3.61 (bs, 2H), 3.50-3.34 (m, 4H), 2.59-2.52 (m, 1H), 2.47-2.33 (m, 4H), 2.21-2.11 (m, 1H), 0.93 (d, J = 6.8 Hz, 6H), 0.72-0.58 (m, 2H), 0.49-0.36 (m, 2H), 4H are missing and/or hidden. MS (m/z): 733.6 (M + H). |
| 14 | 6 | 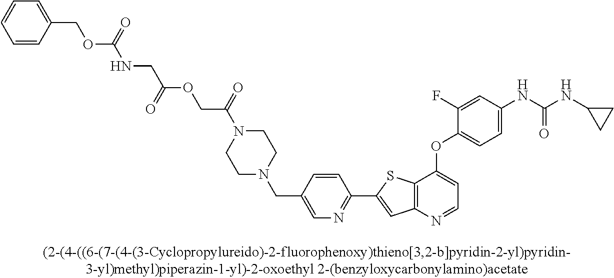 [SMILES: O=C(Nc1ccc(Oc2ccnc3cc(-c4ccc(CN5CCN(C(=O)COC(=O)CNC(=O)OCc6ccccc6)CC5)cn4)sc23)c(F)c1)NC1CC1] | MS (m/z): 768.7 (M + H). |
| 15 | 7 | 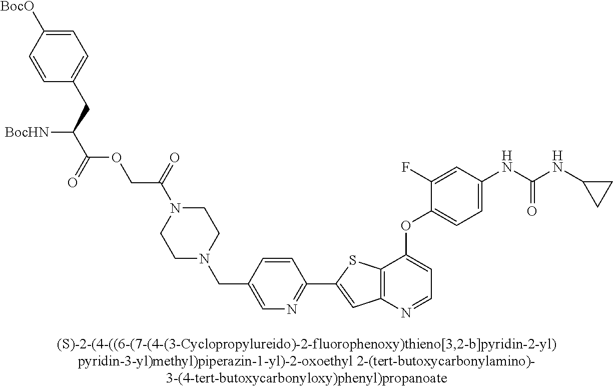 | MS (m/z): 940.2 (M + H). |
| 16 | 8 | 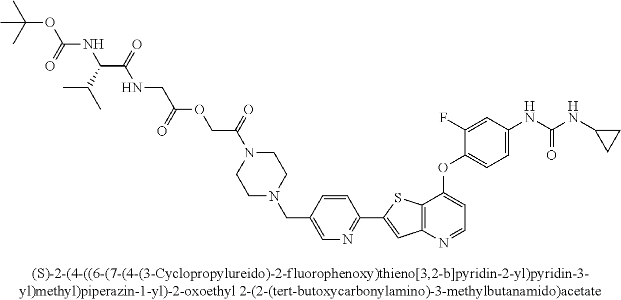 [SMILES: CC(C)[C@H](NC(=O)OC(C)(C)C)C(=O)NCC(=O)OCC(=O)N1CCN(Cc2ccc(-c3cc4nccc(Oc5ccc(NC(=O)NC6CC6)cc5F)c4s3)nc2)CC1] | MS (m/z): 833.2 (M + H). |
| 17 | 9 | 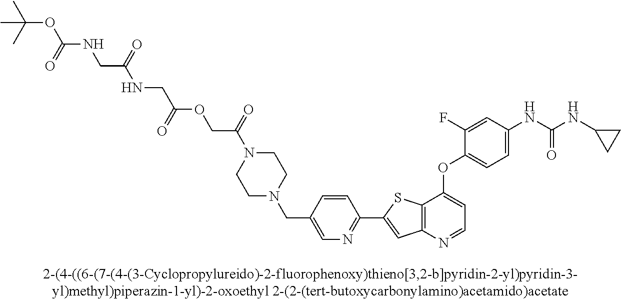 [SMILES: CC(C)(C)OC(=O)NCC(=O)NCC(=O)OCC(=O)N1CCN(Cc2ccc(-c3cc4nccc(Oc5ccc(NC(=O)NC6CC6)cc5F)c4s3)nc2)CC1] | MS (m/z): 791.1 (M + H). |

Compounds 18-19 (examples 10-11) were prepared in one step by coupling hydroxy-compound 4 with the corresponding carboxylic acid (2.5 equiv.) similarly to compound 9 (scheme 4) in the presence of DMAP (1 equiv.) and DCC (4 equiv.) in DMF.

TABLE 2
Characterization of compounds 18-19 (examples 10-11).

| Cpd | Ex. | Structure | Characterization |
|---|---|---|---|
| 18 | 10 | 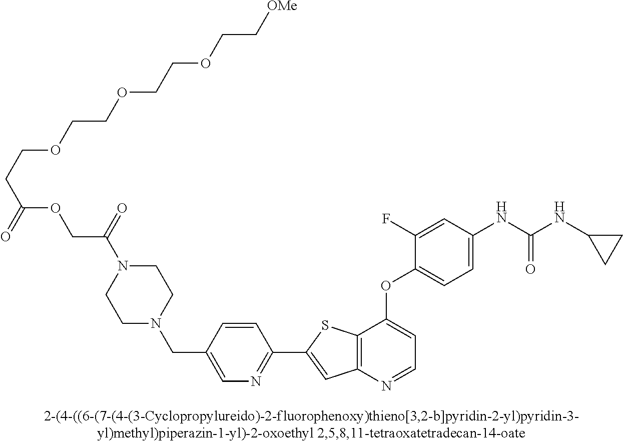 [SMILES: COCCOCCOCCOCCC(=O)OCC(=O)N1CCN(Cc2ccc(-c3cc4nccc(Oc5ccc(NC(=O)NC6CC6)cc5F)c4s3)nc2)CC1] | ¹H NMR (400 MHz, DMSO-d₆) δ (ppm): 8.74 (s, 1H), 8.58 (bd, J = 1.6 Hz, 1H), 8.52 (d, J = 5.5 Hz, 1H), 8.34 (s, 1H), 8.26 (bd, J = 8.0 Hz, 1H), 7.88 (dd, J = 8.1, 2.1 Hz, 1H), 7.73 (dd, J = 13.6, 2.4 Hz, 1H), 7.38 (t, J = 9.0 Hz, 1H), 7.20 (dd, J = 8.8, 1.4 Hz, 1H), 6.65 (dd, J = 5.5, 0.8 Hz, 1H), 6.60 (bd, J = 2.7 Hz, 1H), 4.79 (s, 2H), 3.64 (t, J = 6.4 Hz, 2H), 3.60 (bs, 2H), 3.52-3.35 (m, 16H), 3.23 (s, 3H), 2.61 (t, J = 6.4 Hz, 2H), 2.59-2.52 (m, 1H), 2.47-2.33 (m, 4H), 0.72-0.58 (m, 2H), 0.50-0.36 (m, 2H). MS (m/z): 795.61 (M + H). |
| 19 | 11 | 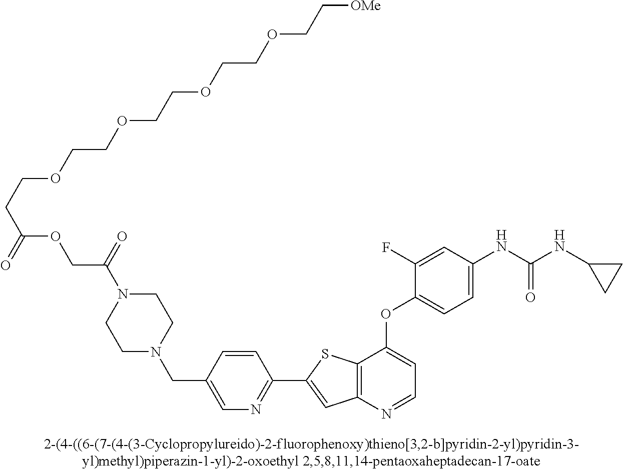 [SMILES: COCCOCCOCCOCCOCCC(=O)OCC(=O)N1CCN(Cc2ccc(-c3cc4nccc(Oc5ccc(NC(=O)NC6CC6)cc5F)c4s3)nc2)CC1] | ¹H NMR (400 MHz, DMSO-d₆) δ (ppm): 8.75 (s, 1H), 8.57 (bd, J = 1.8 Hz, 1H), 8.52 (d, J = 5.5 Hz, 1H), 8.34 (s, 1H), 8.26 (d, J = 8.0 Hz, 1H), 7.88 (dd, J = 8.1, 2.1 Hz, 1H), 7.73 (dd, 13.6, 2.4 Hz, 1H), 7.38 (t, J = 9.0 Hz, 1H), 7.20 (dd, J = 7.4, 1.6 Hz, 1H), 6.65 (d, J = 5.3 Hz, 1H), 6.60 (bd, J = 2.5 Hz, 1H), 4.79 (s, 2H), 3.64 (t, J = 6.4 Hz, 2H), 3.60 (s, 2H), 3.54-3.36 (m, 20H), 3.23 (s, 3H), 2.61 (t, J = 6.4 Hz, 2H), 2.59-2.51 (m, 1H), 2.47-2.33 (m, 4H), 0.72- 0.58 (m, 2H), 0.50-0.36 (m, 2H). MS (m/z): 839.6 (M + H). |

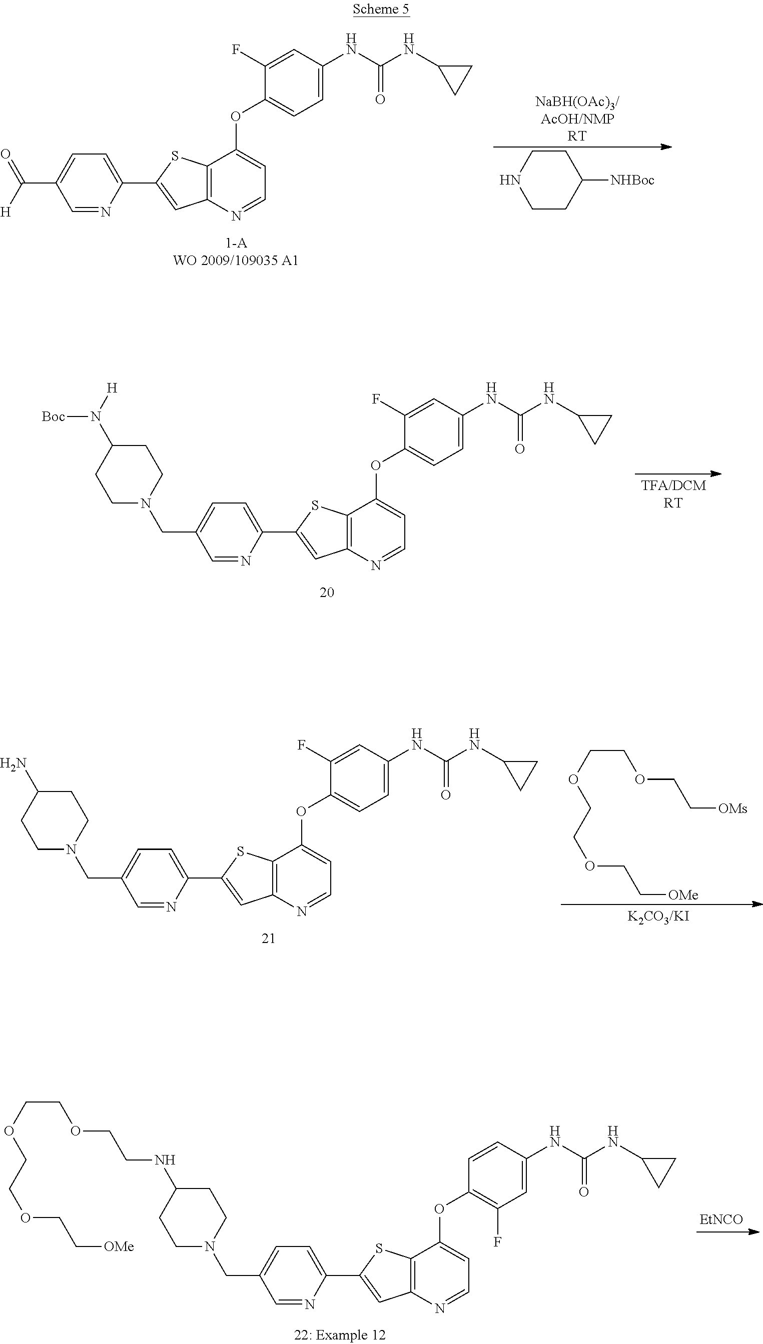

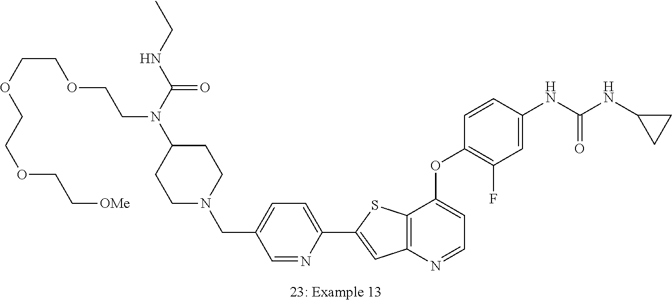 [SMILES: CCNC(=O)N(CCOCCOCCOCCOC)C1CCN(Cc2ccc(-c3cc4nccc(Oc5ccc(NC(=O)NC6CC6)cc5F)c4s3)nc2)CC1]

### Examples 12 and 13

### 1-(4-(2-(5-((4-(2,5,8,11-Tetraoxamidecan-13-ylamino)piperidin-1-yl)methyl)pyridin-2-yl)thieno[3,2-b]pyridin-7-yloxy)-3-fluorophenyl)-3-cyclopropylurea (22) and 1-(4-(2-(5-((4-Ethylaminocarbonyl-(2,5,8,11-tetraoxamidecan-13-ylamino)piperidin-1-yl)methyl)pyridin-2-yl)thieno[3,2-b]pyridin-7-yloxy)-3-fluorophenyl)-3-cyclopropylurea (23)

### Step 1. tert-Butyl 1-((6-(7-(4-(3-cyclopropylureido)-2-fluorophenoxy)thieno[3,2-b]pyridin-2-yl)pyridin-3-yl)methyl)piperidin-4-ylcarbamate (20)

tert-Butyl piperidin-4-ylcarbamate (1.34 g, 6.69 mmol) was added to a solution of aldehyde 1-A (2.0 g, 4.46 mmol) and glacial AcOH (0.250 mL) in NMP (20 mL). The reaction mixture was stirred for 30 min. NaBH(OAc)₃ was then added and the reaction mixture was stirred for an additional 2.5 hours. The reaction mixture was then poured into a saturated aqueous NaHCO₃ solution. A precipitate was formed which was collected by filtration, washed with water and dried. The crude material was purified by column chromatography using a 5 to 20% gradient of MeOH in EtOAc as eluent to afford the title compound 20 (1.45 g, 51.4% yield). MS (m/z): 633.6 (M+1)+

### Step 2. 1-(4-(2-(5-((4-Aminopiperidin-1-yl)methyl)pyridin-2-yl)thieno[3,2-b]pyridin-7-yloxy)-3-fluorophenyl)-3-cyclopropylurea (21)

A solution of the Boc-protected compound 20 in TFA (25 mL) was stirred at RT for 1.5 hours then evaporated. To the residue was added 3N aqueous NaOH solution and the suspension was stirred at RT overnight, collected by filtration, washed with water and dried to afford the title compound 21 (1.177 g, 96% yield). ¹H NMR (400 MHz, DMSO-d₆) δ (ppm): 8.75 (s, 1H); 8.53-8.51 (m, 2H); 8.32 (s, 1H); 8.23 (d, J=8.2 Hz, 1H); 7.84 (dd, J=8.2, 2.2 Hz, 1H); 7.73 (dd, J=13.5, 2.3 Hz, 1H); 7.38 (t, J=9.0 Hz, 1H); 7.20 (dd, J=8.8 1.2 Hz, 1H); 6.64 (d, J=5.5 Hz 1H); 6.61 (d, J=2.3 Hz, 1H); 3.52 (s, 2H); 2.74 (d, J=11.3 Hz, 2H); 2.58-2.52 (m, 1H); 1.99 (t, J=9.8 Hz, 2H); 1.66 (d, J=11.3 Hz, 2H); 1.29-1.20 (m, 2H); 0.68-0.63 (m, 2H); 0.45-0.41 (m, 2H). [Signal of the NH₂-group is not seen; NH₂—CH-signal is obscured by the peak of residual water]. MS (m/z): 533.5 (M+1)+

### Step 3. 1-(4-(2-(5-((4-(2,5,8,11-Tetraoxamidecan-13-ylamino)piperidin-1-yl)methyl)pyridin-2-yl)thieno[3,2-b]pyridin-7-yloxy)-3-fluorophenyl)-3-cyclopropylurea (22)

To a suspension of 21 (0.53 g, 1.0 mmol) in DMF (12 mL) was added K₂CO₃ (276 mg, 2.0 mmol), 2,5,8,11-tetraoxamidecan-13-yl methanesulfonate (430 mg, 1.5 mmol, K. Fukase, et. al. SynLett., 2005, 2342-2346) and potassium iodide (166 mg, 1.0 mmol). The reaction mixture was stirred at 80° C. for 5 hours. The reaction mixture was poured into saturated aqueous solution of NaHCO₃ to form a precipitate that was collected by filtration, rinsed with water, dried and purified by Biotage (MeOH with 2% NH₃/DCM: 10/90-25/75), to afford the title compound 22 (0.27 g, 0.38 mmol, 38% yield) as a beige solid. ¹H NMR (400 MHz, DMSO-d6) δ (ppm): 8.78 (s, 1H), 8.54 (d, J=1.6 Hz, 1H), 8.52 (d, J=5.6 Hz, 1H), 8.34 (s, 1H), 8.26 (d, J=8.0 Hz, 1H), 7.85 (dd, J=8.0, 2.0 Hz, 1H), 7.73 (dd, J=13.6, 2.4 Hz, 1H), 7.38 (t, J=9.0 Hz, 1H), 7.23-7.17 (m, 1H), 6.65 (dd, J=5.2, 0.8 Hz, 1H), 6.61 (brd, 1H), 3.62-3.45 (m, 15H), 3.44-3.38 (m, 2H), 3.23 (s, 3H), 3.05-2.92 (m, 2H), 2.90-2.80 (m, 2H), 2.60-2.50 (m, 1H), 2.06-1.95 (m, 2H), 1.95-1.85 (m, 2H), 1.53-1.38 (m, 2H), 0.68-0.62 (m, 2H), 0.45-0.40 (m, 2H). MS (m/z): 723.44 (M+H).

### Step 2. 1-(4-(2-(5-((4-Ethylaminocarbonyl-(2,5,8,11-tetraoxamidecan-13-ylamino)piperidin-1-yl)methyl)pyridin-2-yl)thieno[3,2-b]pyridin-7-yloxy)-3-fluorophenyl)-3-cyclopropylurea (23)

To a suspension of 22 (60 mg, 0.082 mmol) in THF (3 mL) was added ethyl isocyanate (20 μL, 0.25 mmol) at RT. The reaction mixture was stirred for 3 hours, concentrated and the residue was purified by Biotage (MeOH/DCM: 0/100-20/80), to afford the title compound 23 (49 mg, 0.062 mmol, 75% yield) as a white solid. ¹H NMR (400 MHz, DMSO-d6) δ (ppm): 8.74 (s, 1H), 8.55 (d, J=1.2 Hz, 1H), 8.51 (d, J=5.2 Hz, 1H), 8.33 (s, 1H), 8.24 (d, J=8.0 Hz, 1H), 7.86 (dd, J=8.0, 2.0 Hz, 1H), 7.73 (dd, J=13.6, 2.4 Hz, 1H), 7.38 (t, J=9.0 Hz, 1H), 7.23-7.16 (m, 1H), 6.64 (dd, J=5.2, 0.8 Hz, 1H), 6.60 (d, J=2.4 Hz, 1H), 6.26 (t, J=5.2 Hz, 1H), 3.84-3.70 (m, 1H), 3.55 (s, 2H), 3.52-3.46 (m, 10H), 3.45-3.38 (m, 4H), 3.26-3.19 (m, 2H), 3.21 (s, 3H), 3.05-2.96 (m, 2H), 2.88-2.81 (m, 2H), 2.59-2.50 (m, 1H), 2.08-1.98 (m, 2H), 1.68-1.57 (m, 2H), 1.53-1.46 (m, 2H), 0.99 (t, J=7.2 Hz, 3H), 0.68-0.62 (m, 2H), 0.45-0.40 (m, 2H). MS (m/z): 794.71 (M+H).

Compounds 24-26 (examples 14-16) were synthesized similarly to compound 23 (example 13, scheme 5) by reacting compound 22 (example 12, scheme 5) with acetoxy acetic acid, trifluoroacetic anhydride or methane sulfonyl chloride, respectively.

TABLE 3
Characterization of compounds 24-26 (examples 14-16)

| Cpd | Ex. | Structure | Characterization |
|---|---|---|---|
| 24 | 14 | 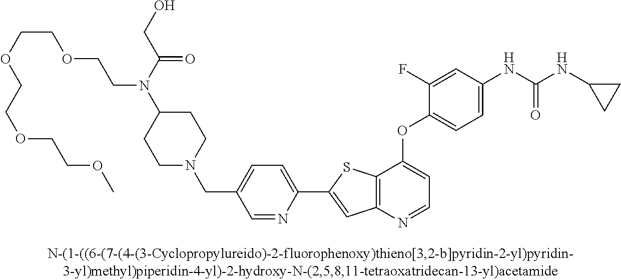 [SMILES: COCCOCCOCCOCCN(C(=O)CO)C1CCN(Cc2ccc(-c3cc4nccc(Oc5ccc(NC(=O)NC6CC6)cc5F)c4s3)nc2)CC1] | ¹H NMR (400 MHz, DMSO-d₆) δ (ppm): 8.70 (s, 1H), 8.56 (brs, 1H), 8.52 (d, J = 5.6 Hz, 1H), 8.33 (s, 1H), 8.24 (d, J = 8.0 Hz, 1H), 7.86 (dd, J = 8.0, 2.0 Hz, 1H), 7.73 (dd, J = 13.6, 2.4 Hz, 1H), 7.38 (t, J = 9.0 Hz, 1H), 7.23-7.16 (m, 1H), 6.64 (dd, J = 5.2, 0.4 Hz, 1H), 6.56 (d, J = 2.8 Hz, 1H), 4.47, 4.30 (t, J = 6.0 Hz, 1H, rotamer), 4.11 (t, J = 6.0 Hz, 1H), 3.95-3.85 (m, 0.5 H, rotamer), 3.57, 3.56 (s, 2H, rotamer), 3.52-3.32 (m, 16H + 0.5H, rotamer), 2.93-2.83 (m, 2H), 2.59- 2.50 (m, 1H), 2.12-1.99 (m, 2H), 1.93-1.68 (m, 2H), 1.62-1.52 (m, 2H), 0.68-0.62 (m, 2H), 0.45-0.40 (m, 2H). MS (m/z): 781.55 (M + 1). |
| 25 | 15 | 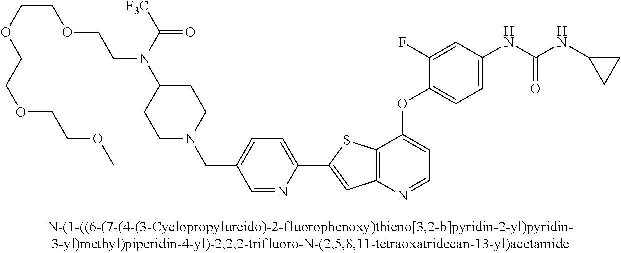 [SMILES: COCCOCCOCCOCCN(C(=O)C(F)(F)F)C1CCN(Cc2ccc(-c3cc4nccc(Oc5ccc(NC(=O)NC6CC6)cc5F)c4s3)nc2)CC1] | ¹H NMR (400 MHz, DMSO-d6) δ (ppm): 8.71 (s, 1H), 8.57 (brs, 1H), 8.52 (d, J = 5.2 Hz, 1H), 8.33 (s, 1H), 8.24 (d, J = 8.0 Hz, 1H), 7.87 (dd, J = 8.0, 2.0 Hz, 1H), 7.73 (dd, J = 13.6, 2.4 Hz, 1H), 7.38 (t, J = 9.0 Hz, 1H), 7.23-7.17 (m, 1H), 6.64 (d, J = 5.2, 0.8 Hz, 1H), 6.60 (d, J = 2.4 Hz, 1H), 3.60-3.45 (m, 17H), 3.41-3.36 (m, 2H), 3.20 (s, 3H), 2.96-2.87 (m, 2H), 2.60-2.50 (m, 1H), 2.13-2.00 (m, 2H), 1.96- 1.84 (m, 2H), 1.69-1.59 (m, 2H), 0.69-0.62 (m, 2H), 0.46-0.41 (m, 2H). MS (m/z): 819.52 (MH)+ |
| 26 | 16 | 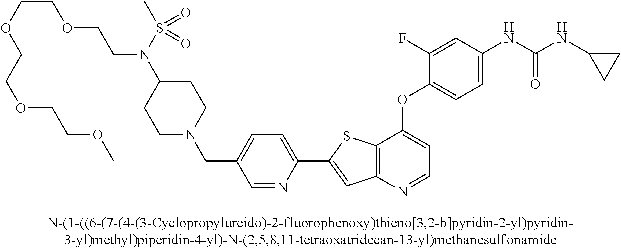 [SMILES: COCCOCCOCCOCCN(C1CCN(Cc2ccc(-c3cc4nccc(Oc5ccc(NC(=O)NC6CC6)cc5F)c4s3)nc2)CC1)S(C)(=O)=O] | ¹H NMR (400 MHz, DMSO-d₆) δ (ppm): 8.73 (s, 1H), 8.55 (d, J = 1.6 Hz, 1H), 8.52 (d, J = 5.2 Hz, 1H), 8.33 (s, 1H), 8.24 (d, J = 8.0 Hz, 1H), 7.86 (dd, J = 8.0, 2.0 Hz, 1H), 7.73 (dd, J = 13.6, 2.4 Hz, 1H), 7.38 (t, J = 9.0 Hz, 1H), 7.23-7.16 (m, 1H), 6.64 (dd, J = 5.2, 0.4 Hz, 1H), 6.59 (d, J = 2.4 Hz, 1H), 3.55 (s, 2H), 3.54-3.45 (m, 13H), 3.43- 3.37 (m, 2H), 3.30-3.25 (m, 2H), 3.21 (s, 3H), 2.95 (s, 3H), 2.91-2.84 (m, 2H), 2.59-2.50 (m, 1H), 2.09- 2.00 (m, 2H), 1.82-1.70 (m, 2H), 1.69-1.62 (m, 2H), 0.68-0.63 (m, 2H), 0.45-0.40 (m, 2H). MS (m/z): 801.50 (M + 1). |

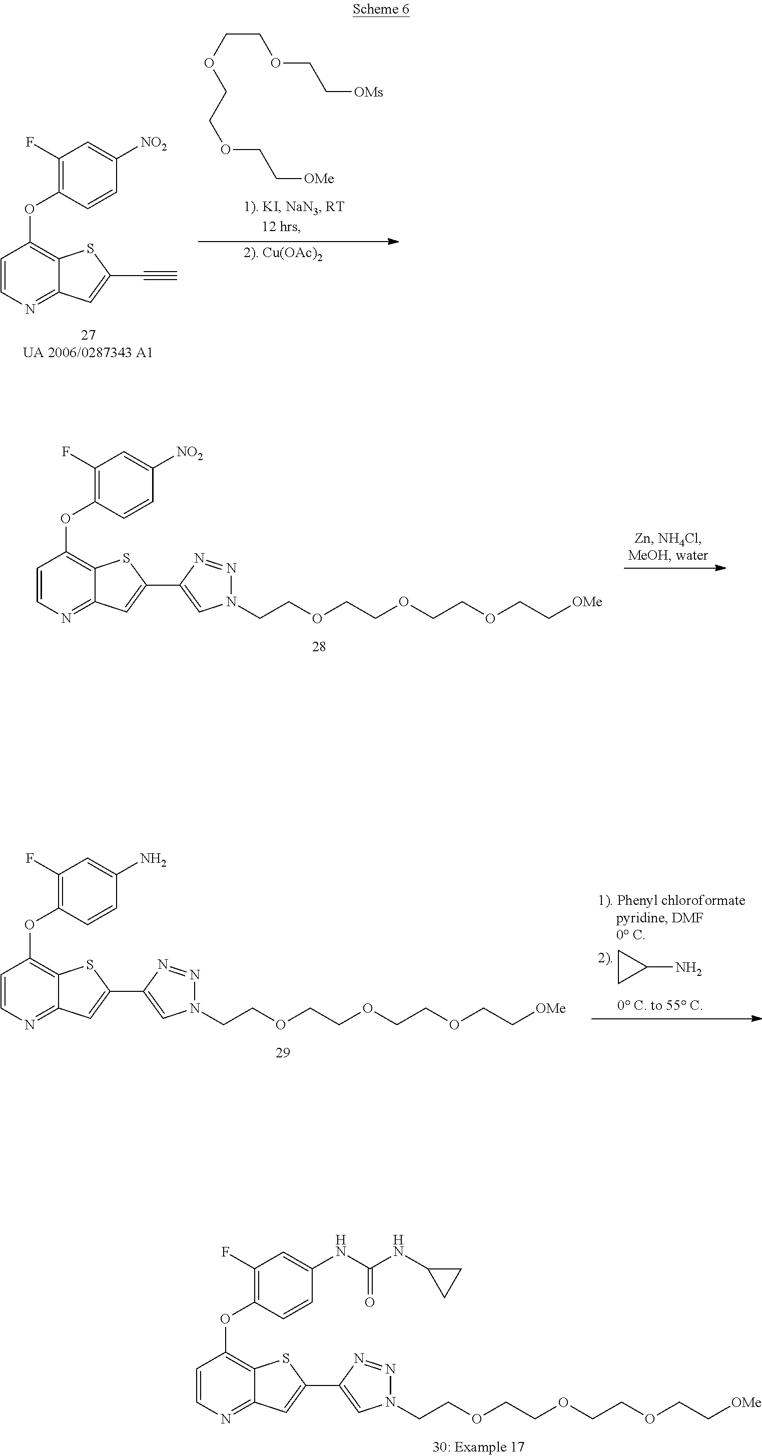

### Example 17

### 1-(4-(2-(1-(2,5,8,11-Tetraoxamidecan-13-yl)-1H-1,2,3-triazol-4-yl)thieno[3,2-b]pyridin-7-yloxy)-3-fluorophenyl)-3-cyclopropylurea (30)

### Step 1: 2-(1-(2,5,8,11-tetraoxamidecan-13-yl)-1H-1,2,3-triazol-4-yl)-7-(2-fluoro-4-nitrophenoxy)thieno[3,2-b]pyridine (28)

To a solution of NaN₃ (49.6 mg, 0.764 mmol) in DMSO (10 mL) was added 2,5,8,11-tetraoxamidecan-13-yl methanesulfonate (102 mg, 1.2 eq, 0.764 mmol) and KI (127 mg, 1.2 eq, 0.764 mmol) and the reaction mixture was stirred for 12 hrs at RT. Compound 27 (200 mg, 0.636 mmol, UA 2006/0287343 A1) and Cu(OAc)₂.H₂O (34.7 mg, 0.3 eq, 0.191 mmol) were added and the reaction mixture was allowed to stir at RT overnight. The reaction mixture was diluted with DCM (100 ml) and then water (50 ml) was added. The mixture was stirred for an additional 20 min; the pases were separated, the organic phase was collected, dried over anhydrous Na₂SO₄, filtered and concentrated to give title compound 28 (348 mg, 100%) as an oil (contaminated with DMSO) which was used crude in the subsequent step. MS (m/z): 548.47 (M+H).

### Step 2: 4-(2-(1-(2,5,8,11-Tetraoxamidecan-13-yl)-1H-1,2,3-triazol-4-yl)thieno[3,2-b]pyridin-7-yloxy)-3-fluoroaniline (29)

To a solution of 28 (348 mg, 0.636 mmol) in MeOH (10 mL) was added ammonium chloride (68 mg, 2 eq, 2.271 mmol) in water (1 mL) and zinc powder (166 mg, 4 eq, 2.54 mmol) and the reaction mixture was heated to reflux for 3 hours. The mixture was cooled to RT, filtered and concentrated. The residue was partitioned between water and DCM and the organic phase was collected, dried over anhydrous Na₂SO₄, filtered and concentrated to afford title compound 29 (298 mg, 91%) as a colourless oil. MS (m/z) 517.67 (M+H)

### Step 3: 1-(4-(2-(1-(2,5,8,11-Tetraoxamidecan-13-yl)-1H-1,2,3-triazol-4-yl)thieno[3,2-b]pyridin-7-yloxy)-3-fluorophenyl)-3-cyclopropylurea (30)

To a stirred solution of 29 (298 mg, 0.576 mmol) and pyridine (0.140 mL, 3 eq, 1.727 mmol) in THF (5 mL)/DMF (1 mL) at 0° C. under nitrogen was added phenyl chloroformate (0.181 mL, 2.5 eq, 1.439 mmol) and the reaction mixture was stirred at 0° C. for 2 hrs. Cyclopropylamine (164 mg, 5 eq, 1.176 mmol) was added and the reaction mixture was heated at 55° C. for 5 hrs. The mixture was cooled to RT, diluted with EtOAc then washed with saturated ammonium chloride solution, saturated NaHCO₃ solution and brine. Finally, the organic phase was dried over anhydrous Na₂SO₄, filtered and concentrated. Trituration of the residue with Et₂O afforded title compound 30 (120 mg, 34%) as a white solid. ¹H NMR (400 MHz, DMSO-d6) δ (ppm): 8.76 (s, 1H), 8.711 (s, 1H), 8.47 (d, J=5.48 Hz, 1H), 7.90 (s, 1H), 7.70 (m, 1H), 7.35 (t, J=9 Hz, 1H), 7.17 (m, 1H), 6.61 (d, J=5.48 Hz, 1H), 6.56 (s, 1H), 4.60 (t, J=5.09 Hz, 2H), 3.85 (t, J=5.09 Hz, 2H), 3.54 (m, 2H), 3.48 (m, 2H), 3.40 (m, 4H), 3.14 (s, 3H), 2.52 (m, 1H), 0.62 (m, 2H), 0.40 (m, 2H). MS (m/z) 601.45.

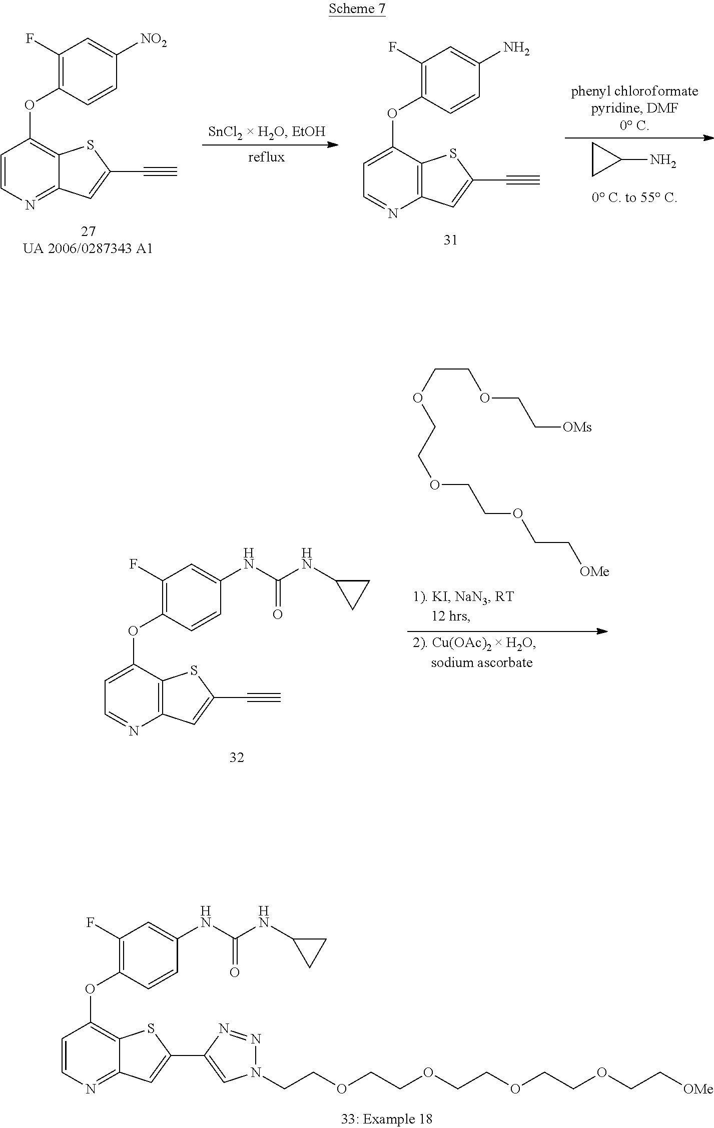

### Example 18

### 1-(4-(2-(1-(2,5,8,11,14-Pentaoxahexadecan-16-yl)-1H-1,2,3-triazol-4-yl)thieno[3,2-b]pyridin-7-yloxy)-3-fluorophenyl)-3-cyclopropylurea (33)

### Step 1: 4-(2-Ethynylthieno[3,2-b]pyridin-7-yloxy)-3-fluoroaniline (31)

To a solution of 27 (120 mg, 0.382 mmol, scheme 6) in EtOH (5 ml) was added SnCl₂×2H₂O (431 mg, 5 eq, 1.91 mmol) and the reaction mixture was heated to reflux for 30 min (Bellamy, F. D.; Ou, K. Tetrahedron Lett. 1984, 25, 839). The mixture was cooled slightly and poured onto ice. A saturated solution of NaHCO₃ and DCM were added and the resultant cloudy mixture was stirred for 15 min. The mixture was then filtered and biphasic filtrate was allowed to separate. The aqueous phase was extracted with additional DCM; the organic phases were combined, dried over anhydrous MgSO₄, filtered and concentrated to afford the title compound 31 (102 mg, 94% yield) that was used in the next step with no additional purification. MS (m/z): 285.17 (M+H).

### Step 2: 1-Cyclopropyl-3-(4-(2-ethynylthieno[3,2-b]pyridin-7-yloxy)-3-fluorophenyl)urea (32)

To a stirred solution of 31 (102 mg, 0.359 mmol) and pyridine (0.058 mL, 2 eq, 0.718 mmol) in THF (5 mL)/DMF (2 mL) mixture at 0° C. under nitrogen was added phenyl chloroformate (0.068 mL, 1.5 eq, 0.538 mmol) and the reaction mixture was stirred at 0° C. for 1 hr. Cyclopropylamine (102 mg, 5 eq, 1.794 mmol) was added and the reaction mixture was heated at 55° C. for an additional 3 hrs. The mixture was then cooled to RT, diluted with EtOAc then washed sequentially with saturated solutions of NH₄Cl, NaHCO₃ and brine. The organic phase was dried over anhydrous Na₂SO₄, filtered and concentrated. The residue was purified via column chromatography (eluent EtOAc to 40% MeOH in EtOAc) to afford the title compound 32 (100 mg, 76% yield) as an off-white powder after trituration with Et₂O. MS (m/z): 368.23 (M+H)

### Step 3: 1-(4-(2-(1-(2,5,8,11,14-Pentaoxahexadecan-16-yl)-1H-1,2,3-triazol-4-yl)thieno[3,2-b]pyridin-7-yloxy)-3-fluorophenyl)-3-cyclopropylurea (33)

To a solution of 2,5,8,11,14-pentaoxahexadecan-16-yl methanesulfonate (108 mg, 2 eq, 0.653 mmol) in DMSO (2 mL) was added sodium azide (42.5 mg, 2 eq, 0.653 mmol) and KI (108 mg, 2 eq, 0.653 mmol) and the reaction mixture was stirred overnight at RT. Compound 32 (120 mg, 0.0.327 mmol, scheme 6), Cu(OAc)₂ (17.8 mg, 0.3 eq, 0.098 mmol) and sodium ascorbate (38.8 mg, 0.6 eq, 0.196 mmol) were added and the pale orange mixture was allowed to stir at for 15 mins. The mixture was poured onto ice and a few drops of NH₄OH was added (˜pH 10). The mixture was extracted with DCM and the organic phase was collected, dried over anhydrous Na₂SO₄, filtered and concentrated. Purification of the residue by column chromatography (EtOAc to 20% MeOH in EtOAc) afforded title compound 33 as a mixture with DMSO. The mixture was dried overnight on a pump and the resultant oil was dissolved in a mixture of acetone/Et₂O and additional Et₂O was added until 33 precipitated as a white solid which was collected by filtration and dried (55 mg, 26% yield). ¹H NMR (400 MHz, DMSO-d6) δ (ppm): 9.27 (s, 1H), 8.78 (s, 1H), 8.49 (d, J=5.47 Hz, 1H), 7.92 (s, 1H), 7.73 (m, 1H), 7.37 (t, J=8.99 Hz, 1H), 7.20 (m, 1H), 7.062 (s, 1H), 6.62 (m, 1H), 4.62 (t, J=4.89 Hz, 2H), 3.88 (t, J=5.087 Hz, 2H), 3.57-3.45 (m, 15H), 3.20 (s, 3H), 2.55 (m 1H), 0.65 (m, 2H), 0.42 (m, 2H). MS (m/z) 645.62

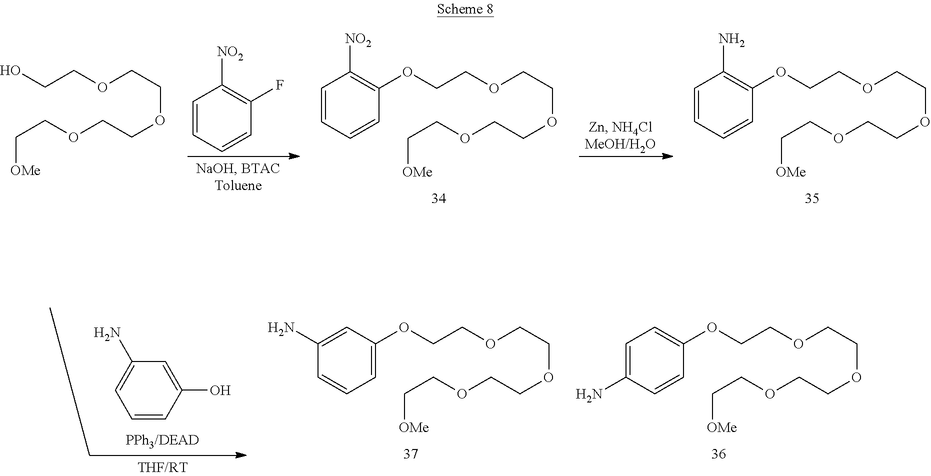

### 2-(2,5,8,11-Tetraoxamidecan-13-yloxy)aniline (35), 4-(2,5,8,11-tetraoxamidecan-13-yloxy)aniline (36) and 3-(2,5,8,11-tetraoxamidecan-13-yloxy)aniline (37)

### Step 1. 13-(2-Nitrophenoxy)-2,5,8,11-tetraoxamidecane (34)

NaOH 5M (10 mL, 50 mmol) was added to a solution of 1-fluoro-2-nitrobenzene (2 g, 14.17 mmol), tetraethyleneglycol monomethyl ether (4.43 g, 21.26 mmol) and benzyltriethylammonium chloride (0.161 g, 0.71 mmol) in toluene (20 mL). The reaction mixture was heated to reflux for 20 h. After cooling to room temperature, the mixture was diluted with water and extracted with EtOAc. The organic layer was washed with brine, dried over anhydrous sodium sulfate, filtered and concentrated. The residue was purified by biotage (SNAP 50 g cartridge; Hex/EtOAc:0/100 to 50/50 over 20 CV), to afford the title compound 34 (4.10 g, 12.45 mmol, 88%) as a yellow oil. MS (m/z): 296.2 (M+H).

### Step 2. 2-(2,5,8,11-Tetraoxamidecan-13-yloxy)aniline (35)

Zinc powder (3.26 g, 49.8 mmol) was added to a solution of 34 (4.10 g, 12.45 mmol), ammonium chloride (1.33 g, 24.90 mmol) in a mixture of MeOH (50 mL) and water (8.50 mL). The reaction mixture was heated to reflux for 1 h. After cooling to room temperature, the reaction mixture was filtered; the solids were washed with MeOH and the filtrate and washings were combined and concentrated. The concentrate was diluted with water and a saturated solution of sodium bicarbonate then extracted with EtOAc. The organic extract was washed brine, dried over sodium sulphate, filtered and evaporated. The residue was purified by biotage (SNAP 100 g cartridge; Hex/EtOAc:50/50 to 0/100 over 20 CV), to afford the title compound 35 (3.26 g, 10.90 mmol, 88%) as a red oil. MS (m/z): 300.2 (M+H).

### 4-(2,5,8,11-Tetraoxamidecan-13-yloxy)aniline (36)

was synthesized by following the procedures described above for the synthesis of compound 35 (scheme 8) using 1-fluoro-4-nitrobenzene as the starting material. MS (m/z): 300.2 (M+H)

### 3-(2,5,8,11-Tetraoxamidecan-13-yloxy)aniline (37)

To a solution of the 3-aminophenol (2.0 g, 18.33 mmol) in THF (40 mL) was added PPh₃ (5.76 g, 21.96 mmol) followed by the addition of DEAD (4.14 g, 23.77 mmol) and a solution of the PEG-OH (3.82 g, 18.33 mmol) in THF (10 mL). The reaction mixture was stirred at RT overnight, the THF was evaporated under reduced pressure and the residue was suspended in ether. The suspension was filtered and the solid (PPh₃P═O) was discarded. The filtrate was evaporated and the residue was purified twice—first time by flash column chromatography, eluent DCM then DCM-MeOH (19:1) then by Biotage (eluent a gradient of EtOAc in DCM from 0 to 20%), to afford the title compound 37 (920 mg, 17%) that was still contaminated with PPh₃P═O and used crude in the following step. MS (m/z): 300.2 (M+H).

Anilines 35-37 were used for the synthesis of compounds 38-40 (examples 19-21, scheme 9 and table 3).

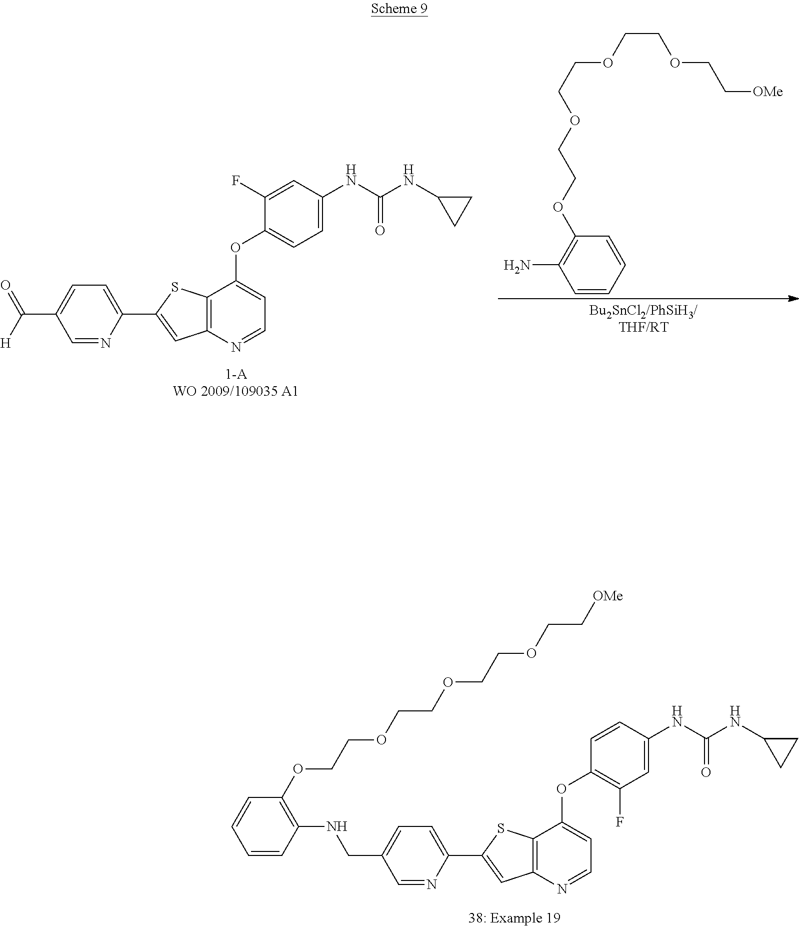 [SMILES: O=Cc1ccc(-c2cc3nccc(Oc4ccc(NC(=O)NC5CC5)cc4F)c3s2)nc1 ; COCCOCCOCCOCCOc1ccccc1N ; COCCOCCOCCOCCOc1ccccc1NCc1ccc(-c2cc3nccc(Oc4ccc(NC(=O)NC5CC5)cc4F)c3s2)nc1]

### Example 19

### 1-(4-(2-(5-((2-(2,5,8,11-Tetraoxamidecan-13-yloxy)phenylamino)methyl)pyridin-2-yl)thieno[3,2-b]pyridin-7-yloxy)-3-fluorophenyl)-3-cyclopropylurea (38)

To a suspension of aldehyde 1-A (0.247 g, 0.551 mmol) and the dibutyltin dichloride (0.057 g, 1.188 mmol) in THF (6 mL) was added a solution of the amine (0.56 g, 1.871 mmol) in THF (6 mL). The combined suspension was treated with a solution of PhSiH₃ (0.165 g, 1.525 mmol) in THF (6 mL) and the reaction mixture was stirred for 24 hrs at RT. To the suspension DMF (3 mL) was added. The mixture turned into a solution and was stirred for an additional 24 hrs. The reaction mixture was evaporated under reduced pressure and the residue was treated with brine to form a precipitate that was collected by filtration and dried. The crude product was purified by flash column chromatography, eluent 5% MeOH in DCM then 10% MeOH in DCM (MeOH contained 2% ammonia) to yield a material which was purified again by flash column chromatography, eluent EtOAc-MeOH (9:1), to provide the title compound 38 (0.058 g, 14.4% yield). Characterization of 38 is provided in the Table 3.

Compounds 39-40 (examples 20-21) were synthesized similarly to compound 38 (scheme 9) by reacting compound 1-A with anilines 36 and 37, respectively.

TABLE 4
Characterization of compounds 38-40 (examples 19-21).

| Cpd | Ex. | Structure | Characterization |
|---|---|---|---|
| 38 | 19 | 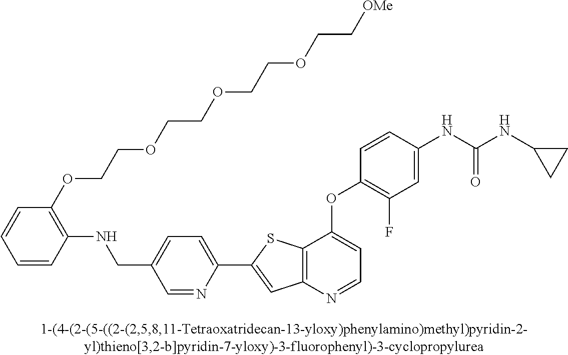 [SMILES: COCCOCCOCCOCCOc1ccccc1NCc1ccc(-c2cc3nccc(Oc4ccc(NC(=O)NC5CC5)cc4F)c3s2)nc1] | ¹H NMR (400 MHz, DMSO-d₆) δ (ppm): 8.71 (s, 1H); 8.62 (d, J = 1.6 Hz, 1H); 8.51 (d, J = 5.5 Hz, 1H); 8.29 (s, 1H); 8.21 (d, J = 8.0 Hz, 1H); 7.88 (dd, J = 8.2, 2.0 Hz, 1H); 7.73 (dd, J = 13.7, 2.5 Hz, 1H); 7.37 (t, J = 9.0 Hz, 1H); 7.20 (br. d, J = 8.8 Hz, 1H); 6.85 (dd, J = 7.8, 1.2 Hz, 1H); 6.71 (dt, J = 7.6, 1.2 Hz 1H); 6.63 (d, J = 5.3 Hz 1H); 6.57-4.69 (m, 3H); 5.65 (t, J = 6.5 Hz, 1H); 4.44 (d, J = 6.3 Hz, 2H); 4.11 (t, 4.5 Hz, 2H); 3.79 (t, J = 3.3 Hz, 2H); 3.78-3.61 (m, 2H); 3.55-3.45 (m, 8H); 3.45- 3.38 (m, 2H); 3.19 (s, 3H); 2.58-2.53 (m, 1H); 0.68-0.63 (m, 2H); 0.45-0.41 (m, 2H). MS (m/z): 732.8 (M + 1). |
| 39 | 20 | 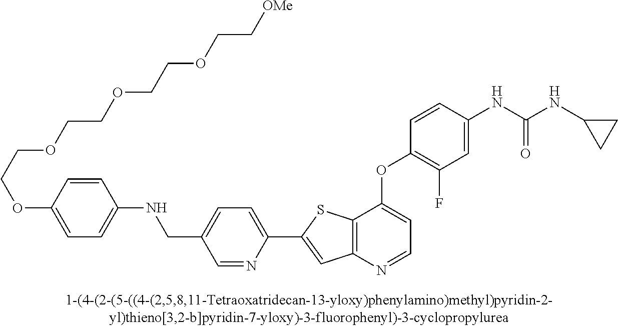 [SMILES: COCCOCCOCCOCCOc1ccc(NCc2ccc(-c3cc4nccc(Oc5ccc(NC(=O)NC6CC6)cc5F)c4s3)nc2)cc1] | ¹H NMR (400 MHz, DMSO-d₆) δ (ppm): 8.72 (s, 1H); 8.62 (d, J = 1.6 Hz, 1H); 8.51 (d, J = 5.5 Hz, 1H); 8.30 (s, 1H); 8.22 (d, J = 8.2 Hz, 1H); 7.89 (dd, J = 8.2, 2.2 Hz, 1H); 7.73 (dd, J = 13.7, 2.5 Hz, 1H); 7.38 (t, J = 9.0 Hz, 1H); 7.20 (br. d, J = 8.6 Hz, 1H); 6.71 (d, J = 9.0 Hz, 2H); 6.63 (d, J = 5.3, 0.8 Hz 1H); 6.59-4.54 (m, 3H); 5.96 (t, J = 6.3 Hz, 1H); 4.30 (d, J = 6.1 Hz, 2H); 3.93- 3.91 (m, 2H); 3.67-3.64 (m, 2H); 3.56-3.47 (m, 10H); 3.41-3.39 (m, 2H); 3.21 (s, 3H); 2.58-2.53 (m, 1H); 0.68-0.63 (m, 2H); 0.45- 0.41 (m, 2H). MS (m/z): 754.6 (M + 1). |
| 40 | 21 | 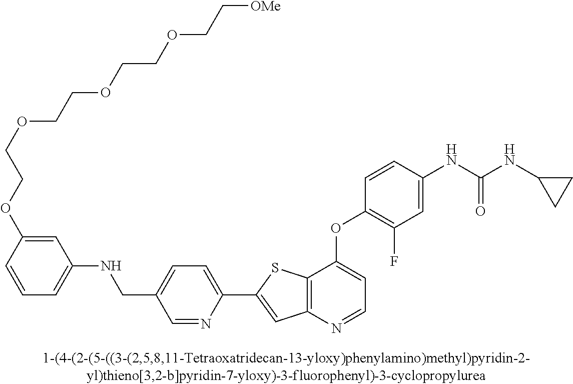 [SMILES: COCCOCCOCCOCCOc1cccc(NCc2ccc(-c3cc4nccc(Oc5ccc(NC(=O)NC6CC6)cc5F)c4s3)nc2)c1] | ¹H NMR (400 MHz, DMSO-d₆) δ (ppm): 8.73 (s, 1H); 8.62 (d, J = 1.6 Hz, 1H); 8.51 (d, J = 5.3 Hz, 1H); 8.30 (s, 1H); 8.23 (d, J = 8.2 Hz, 1H); 7.88 (dd, J = 8.0, 1.8 Hz, 1H); 7.73 (dd, J = 13.5, 2.3 Hz, 1H); 7.37 (t, J = 9.0 Hz, 1H); 7.20 (br. d, J = 8.8 Hz, 1H); 6.95 (t, J = 7.8 Hz, 1H); 6.63 (d, J = 5.5 Hz 1H); 6.59 (d, J = 2.2 Hz 1H); 6.37 (t, J = 6.1 Hz, 1H); 6.23 (bd, J = 8.8 Hz, 1H); 6.14-12 (m, 2H); 4.35 (d, J = 6.1 Hz, 2H); 3.96 (t, 4.1 Hz, 2H); 3.67 (t, J = 4.7 Hz, 2H); 3.56-3.40 (m, 12H); 3.21 (s, 3H); 2.57-2.52 (m, 1H); 0.68- 0.63 (m, 2H); 0.45-0.41 (m, 2H). MS (m/z): 754.8 (M + 1). |

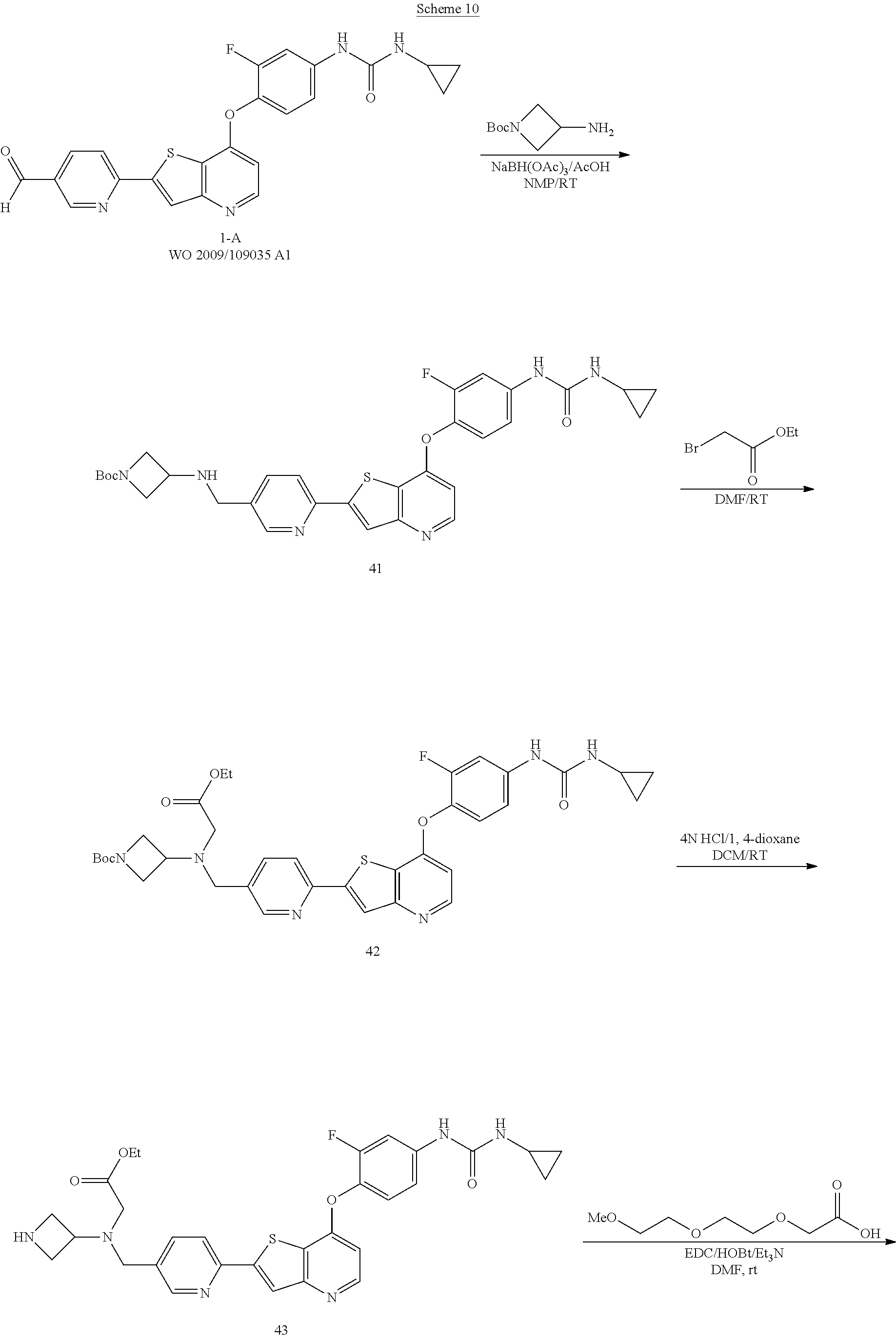

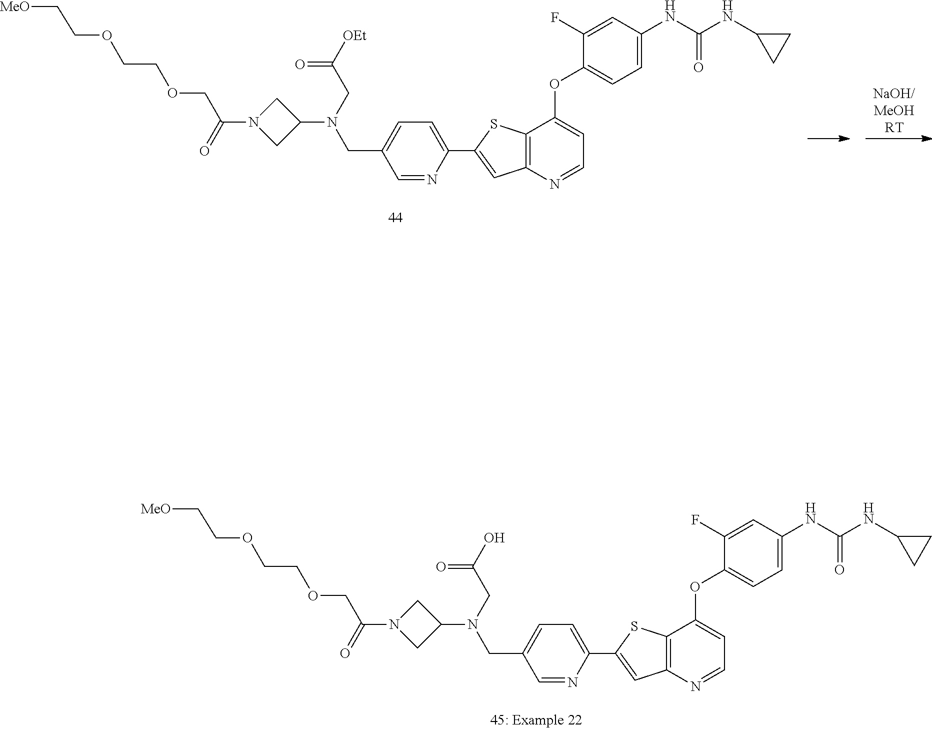 [SMILES: CCOC(=O)CN(Cc1ccc(-c2cc3nccc(Oc4ccc(NC(=O)NC5CC5)cc4F)c3s2)nc1)C1CN(C(=O)COCCOCCOC)C1 ; COCCOCCOCC(=O)N1CC(N(CC(=O)O)Cc2ccc(-c3cc4nccc(Oc5ccc(NC(=O)NC6CC6)cc5F)c4s3)nc2)C1]

### Example 22

### 2-(((6-(7-(4-(3-Cyclopropylureido)-2-fluorophenoxy)thieno[3,2-b]pyridin-2-yl)pyridin-3-yl)methyl)(1-(2-(2-(2-methoxyethoxy)ethoxy)acetyl)azetidin-3-yl)amino)acetic acid (45)

### Step 1. tert-Butyl 3-((6-(7-(4-(3-cyclopropylureido)-2-fluorophenoxy)thieno[3,2-b]pyridin-2-yl)pyridin-3-yl)methylamino)azetidine-1-carboxylate (41)

To a suspension of the aldehyde 1-A (1.0 g, 2.25 mmol, scheme 1) in NMP (12 mL) were added 3-amino-1-N-Boc-azetidine (0.600 g, 3.38 mmol) and acetic acid (0.19 mL, 3.38 mmol) at RT. The reaction mixture was stirred for 30 min; NaBH(OAc)₃ (1.2 g, 5.63 mmol) was added and the stirring was continued for 3 days. The reaction mixture was poured into saturated aqueous solution of NaHCO₃ to form a precipitate that was collected by filtration, rinsed with water, dried and purified via Biotage [linear gradient 2-20%, (methanol/dichloromethane; SiliaFlash 25 g cartridge]. Title compound 41 was obtained as a beige solid (960 mg, 71% yield). ¹H NMR (400 MHz, DMSO-d6) δ (ppm): 8.71 (s, 1H), 8.56 (d, J=1.2 Hz, 1H), 8.51 (d, J=5.2 Hz, 1H), 8.32 (s, 1H), 8.23 (d, J=8.0 Hz, 1H), 7.89 (dd, J=8.0, 2.0 Hz, 1H), 7.73 (dd, J=13.6, 2.4 Hz, 1H), 7.38 (t, J=9.0 Hz, 1H), 7.23-7.18 (m, 1H), 6.64 (dd, J=5.2, 1.2 Hz, 1H), 6.56 (bd, J=2.4 Hz, 1H), 3.98-3.83 (m, 2H), 3.69 (s, 2H), 3.62-3.47 (m, 3H), 2.58-2.51 (m, 1H), 1.36 (s, 9H), 0.68-0.62 (m, 2H), 0.45-0.40 (m, 2H), one NH is missing. MS (m/z): 605.46 (M+H).

### Step 2. tert-Butyl 3-(((6-(7-(4-(3-cyclopropylureido)-2-fluorophenoxy)thieno[3,2-b]pyridin-2-yl)pyridin-3-yl)methyl)(2-ethoxy-2-oxoethyl)amino)azetidine-1-carboxylate (42)

To a solution of 41 (300 mg, 0.496 mmol) in DMF (6 mL) was added ethyl bromoacetate (0.06 mL, 0.546 mmol). The reaction mixture was stirred at RT for 3 days, quenched with water and extracted with DCM. The organic phase was collected, dried over anhydrous Na₂SO₄, filtered and concentrated. The residue was purified by Biotage (SNAP 10 g cartridge; MeOH/DCM: 0/100 to 10/90), to afford the title compound 42 (93 mg, 27% yield) as a beige solid. ¹H NMR (400 MHz, DMSO-d6) δ (ppm): 8.76 (s, 1H), 8.54 (brd, J=1.6 Hz, 1H), 8.52 (d, J=5.6 Hz, 1H), 8.35 (s, 1H), 8.25 (d, J=8.0 Hz, 1H), 7.88 (dd, J=8.0, 2.0 Hz, 1H), 7.73 (dd, J=13.6, 2.4 Hz, 1H), 7.38 (t, J=9.0 Hz, 1H), 7.23-7.18 (m, 1H), 6.65 (dd, J=5.2, 0.8 Hz, 1H), 6.61 (brd, J=2.8 Hz, 1H), 4.05 (q, 7.2 Hz, 2H), 3.93-3.75 (m, 5H), 3.79 (s, 2H), 3.31 (s, 2H), 2.58-2.52 (m, 1H), 1.37 (s, 9H), 1.17 (t, J=7.2 Hz, 3H), 0.68-0.62 (m, 2H), 0.47-0.41 (m, 2H). MS (m/z): 691.64 (M+H).

### Step 3. Ethyl 2-(azetidin-3-yl((6-(7-(4-(3-cyclopropylureido)-2-fluorophenoxy)thieno[3,2-b]pyridin-2-yl)pyridin-3-yl)methyl)amino)acetate (43)

To a solution of 42 (93 mg, 0.135 mmol) in DCM (5 mL) was added 4M HCl in 1,4-dioxane solution (0.17 mL, 0.675 mmol) and the reaction mixture was stirred at RT for 6 h. The mixture was then concentrated to afford the title compound 43 (presumably the hydrochloride salt) as beige solid which was used in the next step with no additional purification. ¹H NMR (400 MHz, DMSO-d6) δ (ppm): 9.13 (s, 1H), 8.98-8.86 (m, 1H), 8.78-8.66 (m, 1H), 8.69 (d, J=6.0 Hz, 1H), 8.63 (d, J=1.2 Hz, 1H), 8.40 (s, 1H), 8.34 (d, J=8.4 Hz, 1H), 7.94 (dd, J=8.0, 2.0 Hz, 1H), 7.77 (dd, J=13.6, 2.4 Hz, 1H), 7.44 (t, J=9.0 Hz, 1H), 7.25-7.21 (m, 1H), 6.92 (d, J=5.2 Hz, 1H), 6.77 (brs, 1H), 4.08 (q, J=7.2 Hz, 2H), 4.15-3.80 (m, 7H), 3.16 (s, 2H), 2.58-2.51 (m, 1H), 1.19 (t, J=7.2 Hz, 3H), 0.68-0.62 (m, 2H), 0.45-0.39 (m, 2H). MS (m/z): 591.58 (M+H).

### Step 4. Ethyl 2-(((6-(7-(4-(3-cyclopropylureido)-2-fluorophenoxy)thieno[3,2-b]pyridin-2-yl)pyridin-3-yl)methyl)(1-(2-(2-(2-methoxyethoxy)ethoxy)acetyl)azetidin-3-yl)amino)acetate (44)

To a solution of 43 (0.136 mmol) in DMF (5 mL) were added 2-[2-(2-methoxyethoxy)ethoxy]acetic acid (49 mg, 0.272 mmol), EDC hydrochloride (52 mg, 0.272 mmol), HOBT monohydrate (31 mg, 0.204 mmol) and triethylamine (95 μL, 0.68 mmol) at RT and the reaction mixture was stirred at RT for 1 h. The mixture was then quenched with water and extracted with EtOAC/MeOH. The organic phase was collected, washed with brine, dried over anhydrous Na₂SO₄, filtered and concentrated. The residue was purified by Biotage (SNAP 10 g cartridge; MeOH/DCM: 0/100 to 20/80), to afford the title compound 44 (65 mg, 64% yield, over 2 steps) as a beige solid. ¹H NMR (400 MHz, DMSO-d6) δ (ppm): 8.82 (s, 1H), 8.55 (d, J=1.6 Hz, 1H), 8.52 (d, J=5.6 Hz, 1H), 8.35 (s, 1H), 8.25 (d, J=8.4 Hz, 1H), 7.89 (dd, J=8.0, 2.0 Hz, 1H), 7.73 (dd, J=13.6, 2.4 Hz, 1H), 7.38 (t, J=9.0 Hz, 1H), 7.24-7.18 (m, 1H), 6.67 (bd, J=2.4 Hz, 1H), 6.65 (dd, J=5.2, 0.8 Hz, 1H), 4.27-4.20 (m, 1H), 4.14-4.02 (m, 5H), 3.97 (s, 2H), 3.96-3.89 (m, 1H), 3.54-3.46 (m, 6H), 3.43-3.37 (m, 2H), 3.22 (s, 3H), 2.59-2.51 (m, 1H), 1.18 (t, J=7.2 Hz, 3H), 0.68-0.62 (m, 2H), 0.45-0.40 (m, 2H). MS (m/z): 751.61 (M+H).

### Step 5. 2-(((6-(7-(4-(3-Cyclopropylureido)-2-fluorophenoxy)thieno[3,2-b]pyridin-2-yl)pyridin-3-yl)methyl)(1-(2-(2-(2-methoxyethoxy)ethoxy)acetyl)azetidin-3-yl)amino)acetic acid (45)

To a solution of 44 (65 mg, 0.0866 mmol) in MeOH (2 mL) was added 1N NaOH (0.17 mL, 0.17 mmol) solution and the reaction mixture was stirred at RT for 24 h. The mixture was then concentrated, diluted with water and the pH was adjusted to 6-7 by addition of 1N HCl. To the resultant suspension was solubilized by addition of MeOH and purified via Biotage [KP-C18-HS 30 g, gradient 20-95% (methanol/water)]. Title compound 45 was obtained as a white solid (32.2 mg, 51% yield). ¹H NMR (400 MHz, DMSO-d6) δ (ppm): 10.91 (brs, 1H), 8.68 (s, 1H), 8.60 (brs, 1H), 8.41 (d, J=5.6 Hz, 1H), 78.22 (s, 1H), 8.16 (d, J=8.0 Hz, 1H), 7.88 (dd, J=8.0, 2.0 Hz, 1H), 7.81 (dd, J=13.6, 2.4 Hz, 1H), 7.22 (t, J=9.0 Hz, 1H), 7.14-7.08 (m, 1H), 6.47 (d, J=5.2 Hz, 1H), 4.25-4.15 (m, 1H), 4.10-4.02 (m, 1H), 4.00-3.87 (m, 2H), 3.96 (s, 2H), 3.84 (s, 2H), 3.78-3.72 (m, 1H), 3.54-3.46 (m, 5H), 3.44-3.36 (m, 3H), 3.21 (s, 3H), 2.58-2.51 (m, 1H), 0.64-0.58 (m, 2H), 0.43-0.39 (m, 2H). [The proton of the carboxy-group is not seen in the spectrum]. MS (m/z): 723.31 (M+H).

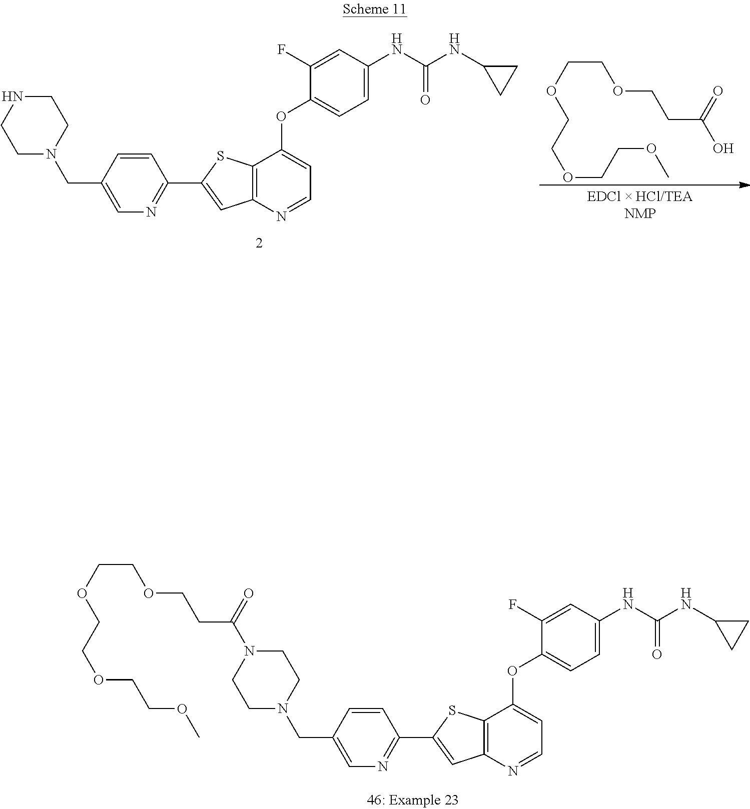

### Example 23

### 1-(4-(2-(5-((4-2,5,8,11-Tetraoxatetradecanepiperazin-1-yl)methyl)pyridin-2-yl)thieno[3,2-b]pyridin-7-yloxy)-3-fluorophenyl)-3-cyclopropylurea (46)

To a solution of compound 2 (0.080 g, 0.15 mmol, scheme 1), 2,5,8,11-tetraoxatetradecane-14-oic acid (0.073 g, 0.31 mmol), and TEA (0.059 g, 0.58 mmol) in NMP (1.5 mL) was added EDCI (0.059 g, 0.31 mmol) and the reaction mixture was stirred at room temperature for 15 h, diluted with water and extracted with EtOAc. The organic layer was washed with a saturated aqueous NaHCO₃ solution, water, and brine; dried over MgSO₄ and concentrated. The residue was purified by flash chromatography on silica gel (eluent EtOAc/MeOH) to afford title compound 46 (0.050 g, 44% yield) as a white powder. ¹H NMR (300 MHz, MeOH-d₄) δ (ppm): 8.62 (d, J=1.5 Hz, 1H), 8.50 (d, J=5.7 Hz, 1H), 8.12 (d, J=7.2 Hz, 1H), 8.11 (s, 1H), 7.96 (dd, J=1.8, 8.1 Hz, 1H), 7.70 (dd, J=2.7, 13.2 Hz, 1H), 7.33 (t, J=8.7 Hz, 1H), 7.25-7.21 (m, 1H), 6.67 (dd, J=1.2, 5.7 Hz, 1H), 3.77 (t, J=6.0 Hz, 2H), 3.72-3.60 (m, 16H), 3.58-3.52 (m, 2H), 3.37 (s, 3H), 2.69 (t, J=6.0 Hz, 2H), 2.64 (tt, J=3.9 Hz, 1H), 2.60-2.54 (m, 4H), 0.84-0.76 (m, 2H), 0.62-0.55 (m, 2H) [Peaks of the two NH protons were not observed]. MS (m/z): 737.2 (M+H)⁺, 759.4 (M+Na)⁺.

Compounds 47-49 (examples 24-24) were prepared in one step by coupling compound 2 (or its HCl salt) with an appropriate acid by following the procedure similar to the one described above for the synthesis of compound 46 (example 23, scheme 11).

TABLE 5
Characterization of compounds 47-49 (examples 24-26)

| Cpd | Ex. | Structure | Characterization |
|---|---|---|---|
| 47 | 24 | 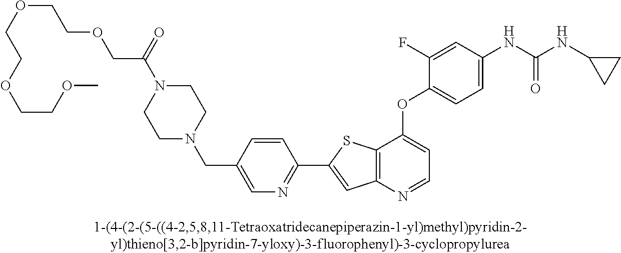 [SMILES: COCCOCCOCCOCC(=O)N1CCN(Cc2ccc(-c3cc4nccc(Oc5ccc(NC(=O)NC6CC6)cc5F)c4s3)nc2)CC1] | ¹H NMR (300 MHz, DMSO-d6) δ (ppm): 8.74 (s, 1H), 8.58 (s, 1H), 8.52 (dd, J = 0.9, 5.4 Hz, 1H), 8.33 (s, 1H), 8.25 (d, J = 8.1 Hz, 1H), 7.88 (dd, J = 1.2, 8.4 Hz, 1H), 7.73 (dd, J = 2.1, 14.4 Hz, 1H), 7.38 (t, J = 9.0 Hz, 1H), 7.24-7.19 (m, 1H), 6.65 (d, J = 5.4 Hz, 1H), 6.61 (brs, 1H), 4.13 (s, 2H), 3.63-3.38 (m, 18H), 3.22 (s, 3H), 2.55 (sep, J = 3.3 Hz, 1H), 2.45- 2.35 (m, 4H), 0.70-0.62 (m, 2H), 0.46-0.40 (m, 2H). MS (m/z): 723.0 (M + H)⁺, 745.3 (M + Na)⁺. |
| 48 | 25 | 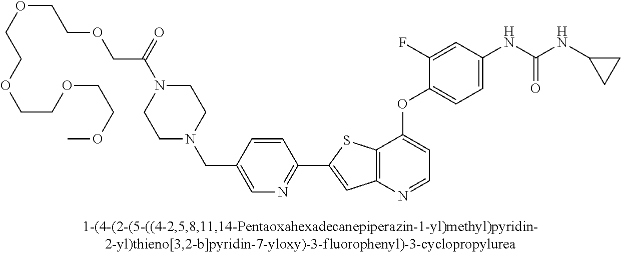 [SMILES: COCCOCCOCCOCCOCC(=O)N1CCN(Cc2ccc(-c3cc4nccc(Oc5ccc(NC(=O)NC6CC6)cc5F)c4s3)nc2)CC1] | ¹H NMR (300 MHz, DMSO-d₆) δ (ppm): 8.76 (brs, 1H), 8.58 (s, 1H), 8.52 (d, J = 5.1 Hz, 1H), 8.33 (s, 1H), 8.25 (d, J = 8.1 Hz, 1H), 7.88 (dd, J = 1.2, 8.4 Hz, 1H), 7.73 (dd, J = 2.4, 14.1 Hz, 1H), 7.38 (t, J = 9.0 Hz, 1H), 7.24-7.19 (m, 1H), 6.65 (d, J = 5.1 Hz, 1H), 6.62 (brs, 1H), 4.13 (s, 2H), 3.63-3.38 (m, 22H), 3.22 (s, 3H), 2.55 (sep, J = 3.3 Hz, 1H), 2.45-2.35 (m, 4H), 0.70- 0.62 (m, 2H), 0.46-0.40 (m, 2H). MS (m/z): 767.3 (M + H)⁺. |
| 49 | 26 | 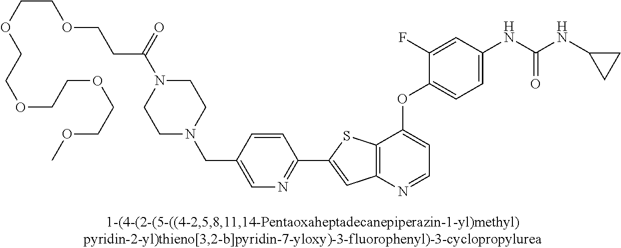 [SMILES: COCCOCCOCCOCCOCCC(=O)N1CCN(Cc2ccc(-c3cc4nccc(Oc5ccc(NC(=O)NC6CC6)cc5F)c4s3)nc2)CC1] | ¹H NMR (300 MHz, MeOH-d₄) δ (ppm): 8.62 (d, J = 1.5 Hz, 1H), 8.50 (d, J = 5.4 Hz, 1H), 8.12 (d, J = 7.2 Hz, 1H), 8.11 (s, 1H), 7.96 (dd, J = 1.8, 8.1 Hz, 1H), 7.70 (dd, J = 2.7, 13.2 Hz, 1H), 7.33 (t, J = 8.7 Hz, 1H), 7.25- 7.21 (m, 1H), 6.67 (d, J = 5.7 Hz, 1H), 3.78 (t, J = 6.0 Hz, 2H), 3.72-3.60 (m, 20H), 3.59-3.52 (m, 2H), 3.37 (s, 3H), 2.69 (t, J = 6.0 Hz, 2H), 2.64 (tt, J = 3.9 Hz, 1H), 2.60-2.54 (m, 4H), 0.84-0.76 (m, 2H), 0.62- 0.55 (m, 2H). [Peaks of the two NH protons were not observed]. MS (m/z): 781.2 (M + H)⁺. |

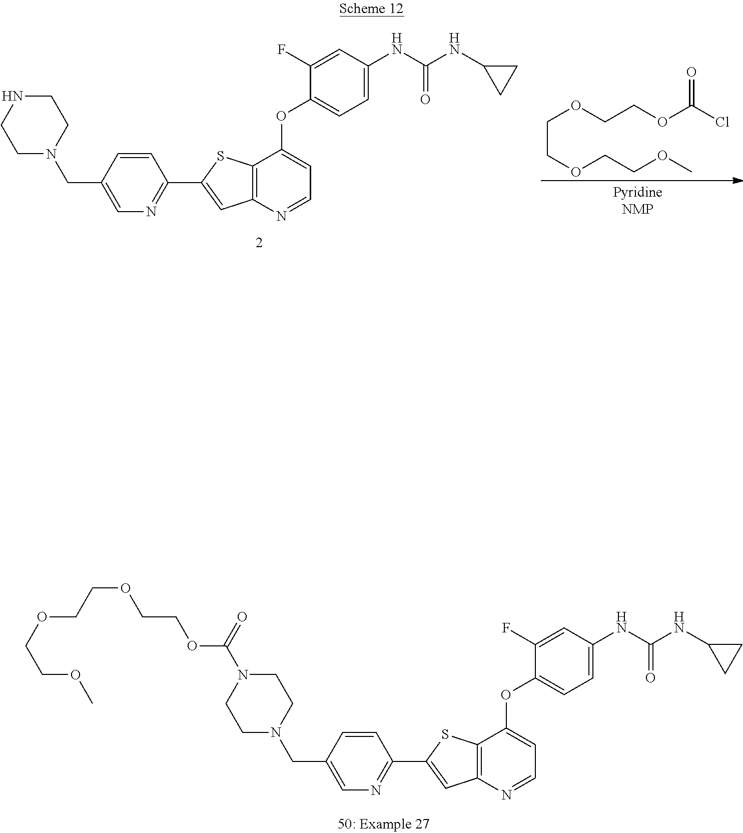 [SMILES: O=C(Nc1ccc(Oc2ccnc3cc(-c4ccc(CN5CCNCC5)cn4)sc23)c(F)c1)NC1CC1 ; COCCOCCOCCOC(=O)Cl ; COCCOCCOCCOC(=O)N1CCN(Cc2ccc(-c3cc4nccc(Oc5ccc(NC(=O)NC6CC6)cc5F)c4s3)nc2)CC1]

### Example 27

### 2-(2-(2-Methoxyethoxy)ethoxy)ethyl 4-((6-(7-(4-(3-cyclopropylureido)-2-fluorophenoxy)thieno[3,2-b]pyridin-2-yl)pyridin-3-yl)methyl)piperazine-1-carboxylate (50)

To a solution of compound 2 (0.080 g, 0.15 mmol, scheme 1) and pyridine (0.072 g, 0.90 mmol) in NMP (1 mL) was added 2-(2-(2-methoxyethoxy)ethoxy)ethyl carbonochloridate (0.16 g, 0.71 mmol). The reaction mixture was stirred at room temperature for 24 h, diluted with saturated NH₄Cl aqueous solution, and extracted with EtOAc-THF (4:1 mixture). The organic layer was collected, washed with saturated NaHCO₃ aqueous solution, water, and brine; dried over MgSO₄ and concentrated. The residue was purified by flash chromatography on silica gel (eluent EtOAc/MeOH) to afford title compound 50 (0.044 g, 40% yield) as an amorphous solid. ¹H NMR (300 MHz, MeOH-d₄) δ (ppm): 8.61 (s, 1H), 8.49 (d, J=5.4 Hz, 1H), 8.12 (d, J=7.8 Hz, 1H), 8.11 (s, 1H), 7.95 (dd, J=2.4, 8.1 Hz, 1H), 7.70 (dd, J=2.4, 12.9 Hz, 1H), 7.33 (t, J=8.7 Hz, 1H), 7.25-7.21 (m, 1H), 6.67 (d, J=5.4 Hz, 1H), 4.26-4.22 (m, 2H), 3.75-3.62 (m, 10H), 3.59-3.52 (m, 6H), 3.37 (s, 3H), 2.64 (tt, J=3.6 Hz, 1H), 2.58-2.48 (m, 4H), 0.84-0.76 (m, 2H), 0.60-0.54 (m, 2H) [Peaks of the two NH protons were not observed]. MS (m/z): 709.3 (M+H)⁺, 731.3 (M+Na)⁺.

Compounds 51-52 (examples 28-29) were prepared in one step by reacting compound 2 with an appropriate chloroformate by following the procedure similar to the one described above for the synthesis of compound 50 (example 27, scheme 12).

TABLE 6
Characterization of compounds 51-52 (examples 28-29)

| Cpd | Ex. | Structure | Characterization |
|---|---|---|---|
| 51 | 28 | 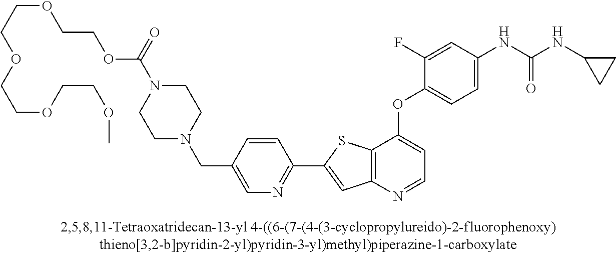 | ¹H NMR (300 MHz, DMSO-d6) δ (ppm): 8.74 (s, 1H), 8.58 (s, 1H), 8.52 (dd, J = 0.9, 5.4 Hz, 1H), 8.33 (s, 1H), 8.25 (d, J = 8.1 Hz, 1H), 7.88 (dd, J = 1.2, 8.4 Hz, 1H), 7.73 (dd, J = 2.1, 14.4 Hz, 1H), 7.38 (t, J = 9.0 Hz, 1H), 7.24-7.19 (m, 1H), 6.65 (d, J = 5.4 Hz, 1H), 6.61 (brs, 1H), 4.13 (s, 2H), 3.63-3.38 (m, 18H), 3.22 (s, 3H), 2.55 (sep, J = 3.3 Hz, 1H), 2.45-2.35 (m, 4H), 0.70-0.62 (m, 2H), 0.46-0.40 (m, 2H). MS (m/z): 723.0 (M + H)⁺, 745.3 (M + Na)^{+.} |
| 52 | 29 | 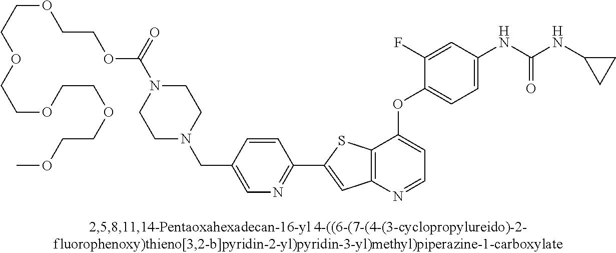 [SMILES: COCCOCCOCCOCCOCCOC(=O)N1CCN(Cc2ccc(-c3cc4nccc(Oc5ccc(NC(=O)NC6CC6)cc5F)c4s3)nc2)CC1] | ¹H NMR (400 MHz, MeOH-d₄) δ (ppm): 8.61 (d, J = 1.5 Hz, 1H), 8.49 (d, J = 5.4 Hz, 1H), 8.12 (d, J = 7.8 Hz, 1H), 8.11 (s, 1H), 7.95 (dd, J = 2.4, 8.1 Hz, 1H), 7.70 (dd, J = 2.7, 13.2 Hz, 1H), 7.33 (t, J = 8.7 Hz, 1H), 7.25-7.21 (m, 1H), 6.67 (dd, J = 1.2, 5.7 Hz, 1H), 4.26-4.22 (m, 2H), 3.75-3.52 (m, 24H), 3.37 (s, 3H), 2.64 (tt, J = 3.6 Hz, 1H), 2.58-2.50 (m, 4H), 0.84-0.76 (m, 2H), 0.60-0.54 (m, 2H) [Peaks of the two NH protons were not observed]. MS (m/z): 797.4 (M + H)⁺, 819.3 (M + Na)⁺. |

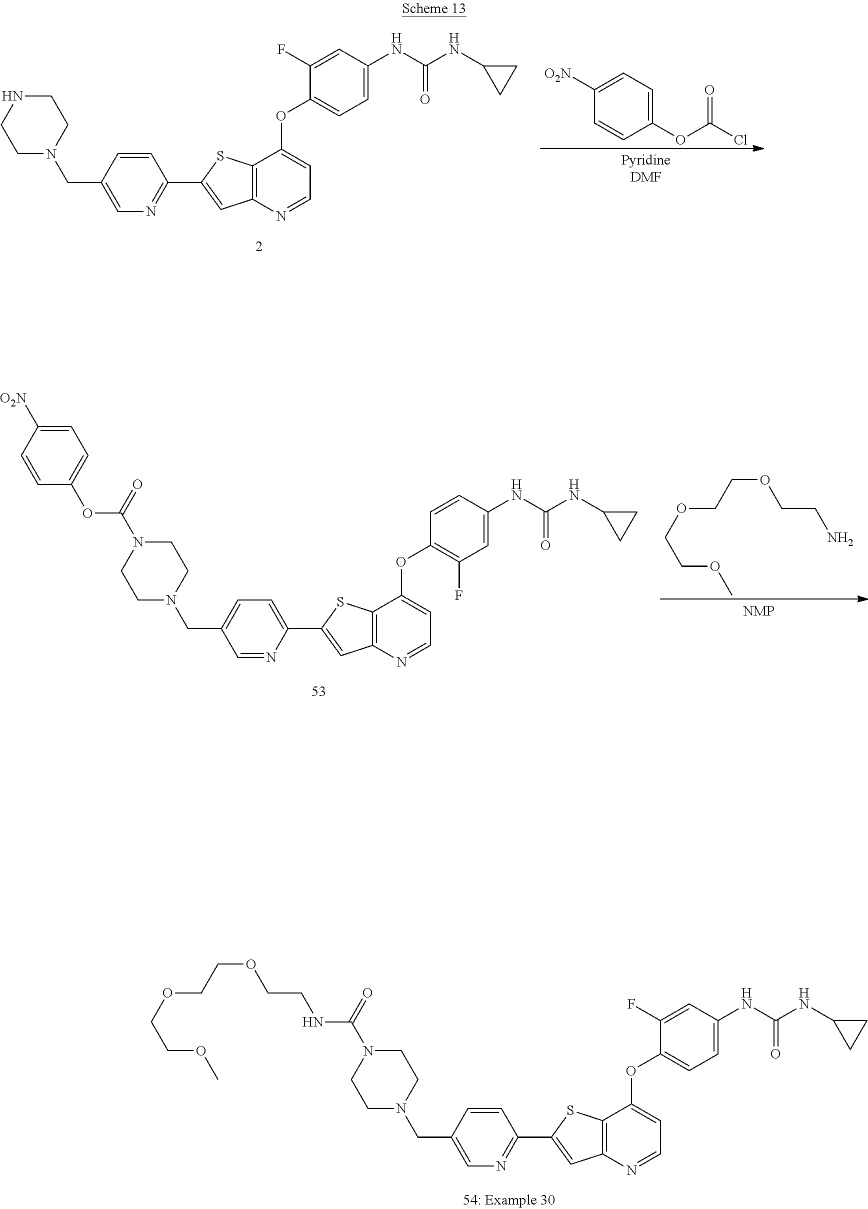

### Example 30

### 4-((6-(7-(4-(3-Cyclopropylureido)-2-fluorophenoxy)thieno[3,2-b]pyridin-2-yl)pyridin-3-yl)methyl)-N-(2-(2-(2-methoxyethoxy)ethoxy)ethyl)piperazine-1-carboxamide (54)

### Step 1. 4-Nitrophenyl 4-((6-(7-(4-(3-cyclopropylureido)-2-fluorophenoxy)thieno[3,2-b]pyridin-2-yl)pyridin-3-yl)methyl)piperazine-1-carboxylate (53)

To a solution of compound 2 (0.50 g, 0.96 mmol, scheme 1) and pyridine (0.11 g, 1.4 mmol) in DMF (4 mL) was added 4-nitrophenyl chlorocarbonate (0.23 g, 1.1 mmol). The reaction mixture was stirred at room temperature for 1 h, diluted with a saturated aqueous NH₄Cl solution and extracted with EtOAc-THF (4:1 mixture). The organic layer was collected, washed with saturated aqueous NaHCO₃ solution, water and brine, dried over MgSO₄ and concentrated. The residue was triturated with t-BuOMe, to afford title compound 53 (0.42 g, 64% yield) as a beige solid. ¹H NMR (300 MHz, DMSO-d₆) δ (ppm): 8.72 (s, 1H), 8.60 (s, 1H), 8.53 (d, J=5.4 Hz, 1H), 8.35 (s, 1H), 8.30-8.26 (m, 3H), 7.91 (d, J=8.1 Hz, 1H), 7.74 (dd, J=2.4, 10.8 Hz, 1H), 7.45 (d, J=8.7 Hz, 2H), 7.39 (t, J=9.0 Hz, 1H), 7.21 (d, J=9.0 Hz, 1H), 6.66 (d, J=5.1 Hz, 1H), 6.58 (s, 1H), 3.65 (brs, 4H), 3.49 (brs, 2H), 3.34 (brs, 4H), 2.58-2.54 (m, 1H), 0.70-0.63 (m, 2H), 0.47-0.43 (m, 2H).

### Step 2. 4-((6-(7-(4-(3-Cyclopropylureido)-2-fluorophenoxy)thieno[3,2-b]pyridin-2-yl)pyridin-3-yl)methyl-N-(2-(2-(2-methoxyethoxy)ethoxy)ethyl)piperazine-1-carboxamide (54)

To a solution of 53 (0.10 g, 0.15 mmol) in NMP (4 mL) was added 2-(2-(2-methoxyethoxy)ethoxy)ethanamine (0.073 g, 45 mmol). The resultant mixture was stirred at 70° C. for 32 h, diluted with saturated aqueous NH₄Cl solution and extracted with EtOAc-THF (4:1 mixture). The organic layer was collected, washed with a saturated NaHCO₃ aqueous solution, water and brine, dried over MgSO₄ and concentrated. The residue was purified by flash chromatography on silica gel (EtOAc/MeOH) to afford title compound 54 (0.058 g, 56% yield) as an amorphous solid. ¹H NMR (300 MHz, MeOH-d₄) δ (ppm): 8.61 (d, J=1.8 Hz, 1H), 8.49 (d, J=5.4 Hz, 1H), 8.12 (d, J=7.8 Hz, 1H), 8.11 (s, 1H), 7.95 (dd, J=2.4, 8.1 Hz, 1H), 7.70 (dd, J=2.7, 13.2 Hz, 1H), 7.33 (t, J=8.7 Hz, 1H), 7.25-7.21 (m, 1H), 6.67 (dd, J=1.2, 5.4 Hz, 1H), 3.70-3.61 (m, 8H), 3.57-3.52 (m, 4H), 3.50-3.43 (m, 4H), 3.40-3.25 (m, 5H), 2.64 (tt, J=3.6 Hz, 1H), 2.62-2.49 (m, 4H), 0.82-0.76 (m, 2H), 0.60-0.54 (m, 2H). [Peaks of the two NH protons were not observed]. MS (m/z): 708.4 (M+H).

Compounds 55-56 (examples 31-32) were prepared in one step by reacting compound 53 with an appropriate amine by following the procedure similar to the one described above for the synthesis of compound 54 (example 30, scheme 13).

TABLE 7
Characterization of compounds 55-56 (examples 31-32)

| Cpd | Ex. | Structure | Characterization |
|---|---|---|---|
| 55 | 31 | 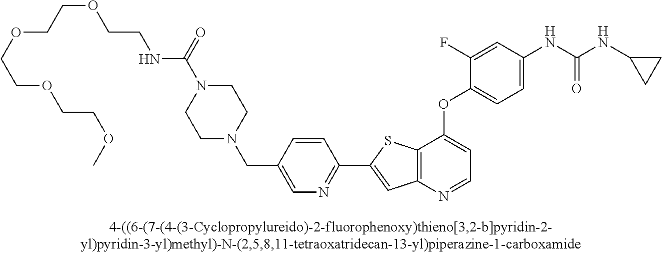 [SMILES: COCCOCCOCCOCCNC(=O)N1CCN(Cc2ccc(-c3cc4nccc(Oc5ccc(NC(=O)NC6CC6)cc5F)c4s3)nc2)CC1] | ¹H NMR (300 MHz, MeOH-d₄) δ (ppm): 8.61 (d, J = 1.8 Hz, 1H), 8.49 (d, J = 5.4 Hz, 1H), 8.12 (d, J = 7.8 Hz, 1H), 8.11 (s, 1H), 7.96 (dd, J = 2.1, 8.1 Hz, 1H), 7.70 (dd, J = 2.4, 13.2 Hz, 1H), 7.34 (t, J = 8.7 Hz, 1H), 7.25-7.21 (m, 1H), 6.68 (dd, J = 0.9, 5.4 Hz, 1H), 3.70-3.61 (m, 12H), 3.60- 3.52 (m, 4H), 3.48-3.42 (m, 4H), 3.38-3.34 (m, 5H), 2.64 (tt, J = 3.6 Hz, 1H), 2.62-2.49 (m, 4H), 0.82-0.76 (m, 2H), 0.60- 0.54 (m, 2H). Peaks of the three NH protons were not observed. MS (m/z): 752.4 (M + H). |
| 56 | 32 | 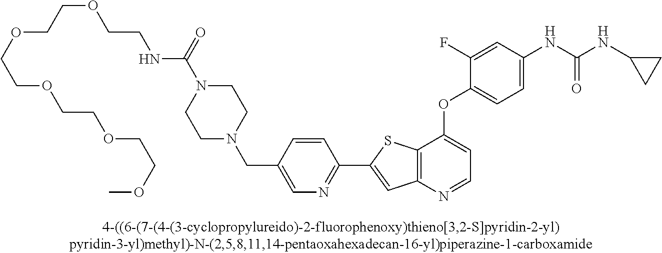 [SMILES: COCCOCCOCCOCCOCCNC(=O)N1CCN(Cc2ccc(-c3cc4nccc(Oc5ccc(NC(=O)NC6CC6)cc5F)c4s3)nc2)CC1] | ¹H NMR (300 MHz, MeOH-d₄) δ (ppm): 8.61 (d, J = 1.8 Hz, 1H), 8.50 (d, J = 6.0 Hz, 1H), 8.13 (d, J = 7.8 Hz, 1H), 8.11 (s, 1H), 7.96 (dd, J = 2.1, 8.4 Hz, 1H), 7.70 (dd, J = 2.4, 13.2 Hz, 1H), 7.33 (t, J = 8.7 Hz, 1H), 7.25-7.21 (m, 1H), 6.67 (dd, J = 1.2, 5.4 Hz, 1H), 3.70-3.60 (m, 16H), 3.57- 3.52 (m, 4H), 3.50-3.43 (m, 4H), 3.38-3.34 (m, 5H), 2.64 (tt, J = 3.6 Hz, 1H), 2.62-2.49 (m, 4H), 0.82-0.76 (m, 2H), 0.60- 0.54 (m, 2H). Peaks of the three NH protons were not observed. MS (m/z): 796.4 (M + H). |

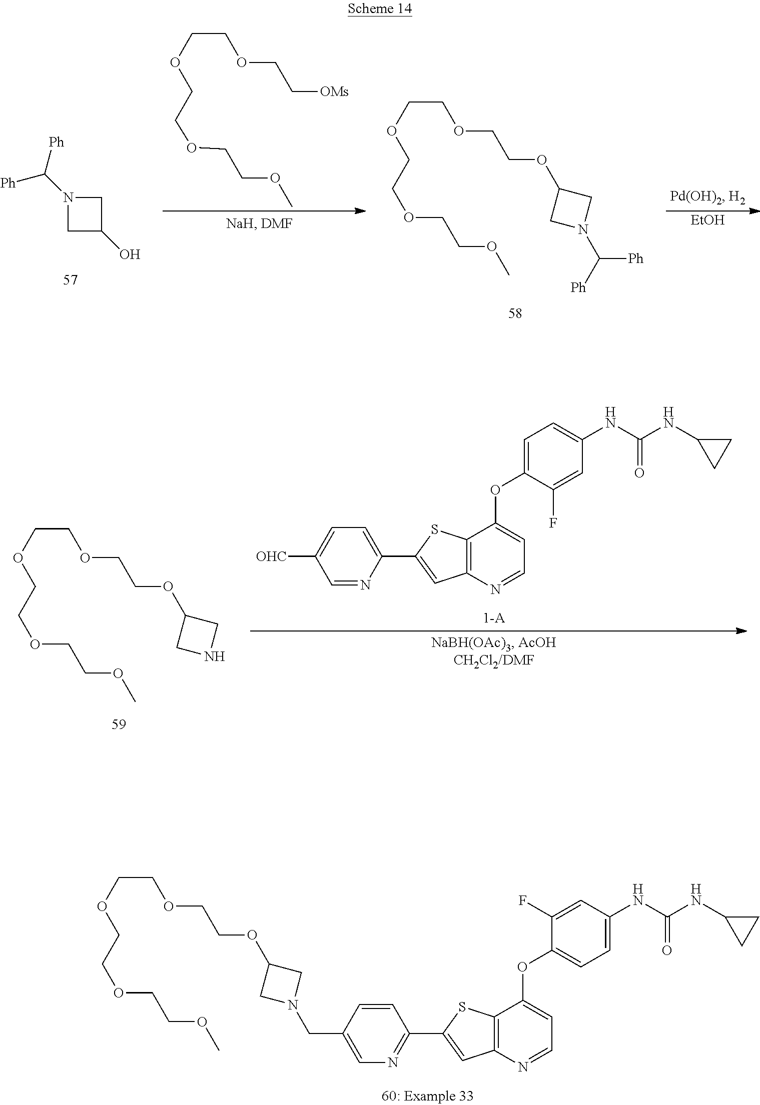

### Example 33

### 1-(4-(2-(5-((3-(2,5,8,11-tetraoxamidecan-13-yloxy)azetidin-1-yl)methyl)pyridin-2-yl)thieno[3,2-b]pyridin-7-yloxy)-3-fluorophenyl)-3-cyclopropylurea (60)

### Step 1. 3-(2,5,8,11-Tetraoxamidecan-13-yloxy)-1-benzhydrylazetidine (58)

To a solution of 1-(diphenylmethyl)-3-hydroxyazetidine (57) (0.66 g, 2.75 mmol) in DMF (6 mL) was added NaH (60% in mineral oil, 0.120 g, 2.9 mmol) at 0° C. and the reaction mixture was stirred for 15 min. Then a solution of 2,5,8,11-tetraoxamidecan-13-yl methanesulfonate (0.715 g, 2.5 mmol, K. Fukase, et. al. SynLett., 2005, 2342-2346) in DMF (2.5 mL) at 0° C. was added and the reaction mixture was stirred at 60° C. for 2.5 hr, quenched with water and extracted with EtOAc. The organic extract was washed with brine and dried over MgSO₄ then concentrated. The crude product was purified by flash chromatography on silica gel (Hexane/EtOAc:50/50 to 0/100) to afford title compound 58 (0.76 g, 71% yield) as a yellow oil. The material was used in the next step without characterization.

### Step 2. 3-(2,5,8,11-Tetraoxamidecan-13-yloxy)azetidine (59)

The mixture of 58 (0.76 g, 1.78 mmol) and Pd(OH)₂ (0.08 g) in EtOH (7 mL) was stirred at 70° C. for 5 hr under hydrogen. The reaction mixture was then filtered through a celite pad, rinsed with EtOH and concentrated. The residue was dissolved in 1N HCl aq and the acidic solution was washed with EtOAc. The washings were discarded and the aqueous layer was treated with 5N NaOH and extracted with DCM/MeOH. The organic extract was washed with brine, dried over MgSO₄ and concentrated to afford title compound 59 (0.40 g, 85% yield) as an orange oil. ¹H NMR (300 MHz, CDCl₃) δ (ppm): 4.91 (brs, 1H), 4.94 (m, 1H), 4.37-4.25 (m, 2H), 4.12-4.03 (m, 2H), 3.70-3.50 (m, 16H), 3.95 (s, 3H).

### Step 3. 1-(4-(2-(5-((3-(2,5,8,11-tetraoxamidecan-13-yloxy)azetidin-1-yl)methyl)pyridin-2-yl)thieno[3,2-b]pyridin-7-yloxy)-3-fluorophenyl)-3-cyclopropylurea (60)

To a suspension of aldehyde 1-A (0.3 g, 0.669 mmol, WO 2009/109035 A1) in a mixture of DCM (9 mL) and DMF (2 mL) were added 59 (0.400 g, 1.54 mmol) and acetic acid (0.09 mL, 1.68 mmol) at room temperature. The reaction mixture was stirred for 1 hr, treated with NaBH(OAc)₃ (0.425 g, 2.00 mmol) and stirred overnight, then quenched by addition of saturated NaHCO₃ solution and extracted with DCM/MeOH. The organic extract was washed with brine and dried over MgSO₄ then concentrated. The residue was purified by flash chromatography on silica gel (DCM/MeOH) to afford title compound 60 (0.051 g, 11% yield) as a beige amorphous solid. ¹H NMR (300 MHz, DMSO-d₆) δ (ppm): 8.69 (s, 1H), 8.54-8.52 (m, 1H), 8.52 (d, J=5.1 Hz, 1H), 8.32 (s, 1H), 8.23 (d, J=8.1 Hz, 1H), 7.83 (dd, J=8.1, 2.4 Hz, 1H), 7.73 (dd, J=13.5, 2.4 Hz, 1H), 7.38 (t, J=9.0 Hz, 1H), 7.22-7.17 (m, 1H), 6.64 (d, J=5.4 Hz, 1H), 6.56 (d, J=3.0 Hz, 1H), 4.10 (t, J=5.7 Hz, 1H), 3.66 (s, 2H), 3.60-3.35 (m, 18H), 3.22 (s, 3H), 2.96-2.86 (m, 2H), 2.60-2.45 (m, 1H), 0.70-0.62 (m, 2H), 0.48-0.40 (m, 2H).

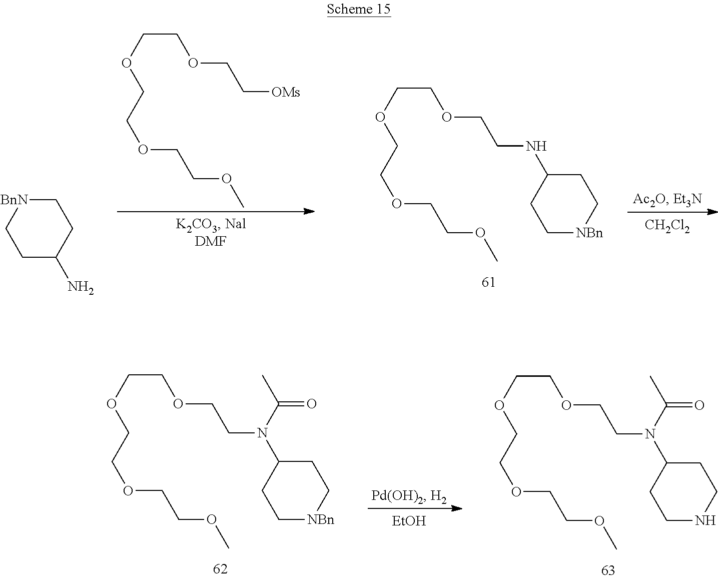

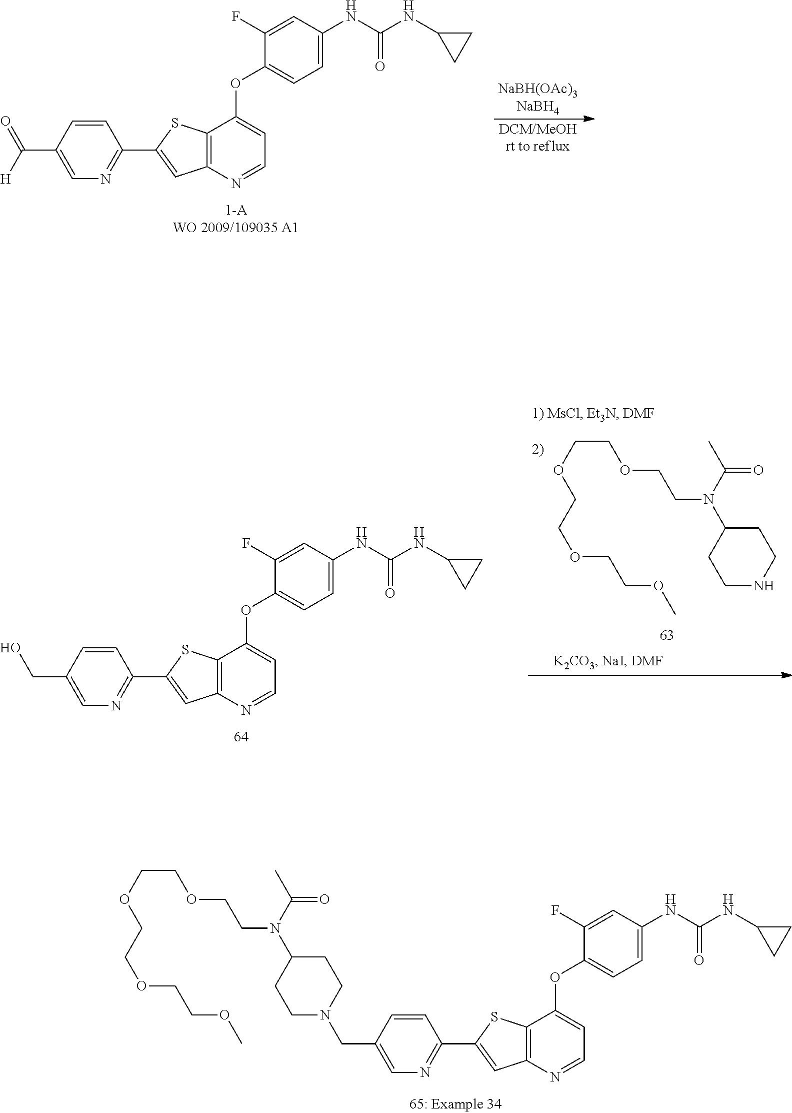 [SMILES: O=Cc1ccc(-c2cc3nccc(Oc4ccc(NC(=O)NC5CC5)cc4F)c3s2)nc1 ; "DCM/MeOHrt to reflux" ; O=C(Nc1ccc(Oc2ccnc3cc(-c4ccc(CO)cn4)sc23)c(F)c1)NC1CC1 ; COCCOCCOCCOCCN(C(C)=O)C1CCNCC1 ; COCCOCCOCCOCCN(C(C)=O)C1CCN(Cc2ccc(-c3cc4nccc(Oc5ccc(NC(=O)NC6CC6)cc5F)c4s3)nc2)CC1]

### Example 34

### N-(1-((6-(7-(4-(3-Cyclopropylureido)-2-fluorophenoxy)thieno[3,2-b]pyridin-2-yl)pyridin-3-yl)methyl)piperidin-4-yl)-N-(2,5,8,11-tetraoxamidecan-13-yl)acetamide (65)

### Step 1. 1-Benzyl-N-(2,5,8,11-tetraoxamidecan-13-yl)piperidin-4-amine (61)

To a solution of 2,5,8,11-tetraoxamidecan-13-yl methanesulfonate (0.715 g, 2.5 mmol, K. Fukase, et. al. SynLett., 2005, 2342-2346) in DMF (5 mL) were added NaI (0.375 g, 2.5 mmol), 4-amino-1-benzylpiperidine (0.95 g, 5.00 mmol) and K₂CO₃ (0.83 g, 6.00 mmol) at 0° C. The reaction mixture was stirred at 60° C. overnight, diluted with water and extracted with EtOAc. The organic layer was washed with brine, dried over MgSO₄ and concentrated to afford title compound 61 (0.87 g,) as a brown oil, which was used in the next step without further purification and characterization.

### Step 2. N-(1-Benzylpiperidin-4-yl)-N-(2,5,8,11-tetraoxamidecan-13-yl)acetamide (62)

To a solution of 61 (0.87 g) in DCM (20 mL) were added TEA (1.4 mL, 10.0 mmol) and Ac₂O (0.8 mL, 7.5 mmol) at room temperature. The reaction mixture was stirred overnight, treated with saturated NaHCO₃ solution and extracted with DCM. The organic extract was washed with brine, dried over MgSO₄ and concentrated. The residue was purified by flash chromatography on silica gel (DCM/MeOH:95/5) to afford title compound 62 (0.55 g) as an orange oil which was used in the next step without further purification and characterization.

### Step 3. N-(piperidin-4-yl)-N-(2,5,8,11-tetraoxamidecan-13-yl)acetamide (63)

The mixture of 62 (0.55 g) and Pd(OH)₂ (0.06 g) in EtOH (6 mL) was stirred at 60° C. for 24 hr under hydrogen. The reaction mixture was then filtered through a celite pad, rinsed with EtOH and concentrated to afford title compound 63 (0.41 g, 49% yield over 3 steps) as an orange oil, which was used in the next step without further purification. ¹H NMR (300 MHz, CDCl₃) δ (ppm): 3.70-3.50 (m, 15H), 3.47-3.40 (m, 2H), 3.38 (s, 3H), 3.23-3.15 (m, 2H), 2.75-2.56 (m, 2H), 2.14 (s, H), 2.13 (s, H), 1.90-1.50 (m, 4H).

### Step 4. 1-cyclopropyl-3-(3-fluoro-4-(2-(5-(hydroxymethyl)pyridin-2-yl)thieno[3,2-b]pyridin-7-yloxy)phenyl)urea (64)

To a suspension of 1-A (5.60 g, 12.49 mmol) in a mixture of DCM (200 mL)/MeOH (20 mL) in a 1 L round-bottomed flask was added sodium triacetoxyborohydride (5.29 g, 24.97 mmol). The reaction mixture was stirred at RT for 5 h. More sodium triacetoxyborohydride (5.29 g, 24.97 mmol) was added and the mixture was stirred at RT for 16 h. Then NaBH₄ (2 g, 52.9 mmol) was added to the reaction mixture that was stirred at RT for 24 h. Finally, more NaBH₄ (2 g, 52.9 mmol) was added and the reaction mixture was heated to reflux for 5 h, then cooled to RT, concentrated, quenched with 10% HCl (100 mL), and neutralized slowly with a saturated aqueous solution of NaHCO₃ (200 mL) to give a grey precipitate. The suspension was shaken for 15 min and the solid was collected by filtration, rinsed with water (2×25 mL) and dried under high vacuum to afford the title compound 64 (5.34 g, 11.85 mmol, 94% yield) as a light grey solid. MS (m/z): 451.5 (M+H). The material was used in the next step with no additional purification.

### Step 5. N-(1-((6-(7-(4-(3-cyclopropylureido)-2-fluorophenoxy)thieno[3,2-b]pyridin-2-yl)pyridin-3-yl)methyl)piperidin-4-yl)-N-(2,5,8,11-tetraoxamidecan-13-yl)acetamide (65)

To a solution of 64 (0.167 g, 0.37 mmol) in DMF (15 mL) at 0° C. were added TEA (0.26 mL, 1.88 mmol) and methanesulfonyl chloride (0.12 mL, 1.52 mmol). The reaction mixture was stirred for 30 min and poured into water to form a precipitate that was collected by filtration, rinsed with water and dried to give a beige powder—(presumably the mesylate derivative of 64 which was not characterized).

To a solution of the beige powder (0.20 g, 0.37 mmol) in DMF (4 mL) were added K₂CO₃ (0.21 g, 1.48 mmol), 63 (0.42 g, 1.22 mmol) and NaI (0.011 g, 0.074 mmol) at 0° C. The reaction mixture was stirred at RT for 2 hrs, diluted with water and extracted with EtOAc/MeOH. The extract was washed with brine, dried over MgSO₄ and concentrated. The residue was purified by flash chromatography on silica gel (DCM/MeOH) to afford title compound 65 (0.107 g, 38% yield over 2 steps) as a white solid. ¹H NMR (300 MHz, DMSO-d₆) δ (ppm): 8.69 (s, 1H), 8.56 (brs, 1H), 8.52 (d, J=5.4 Hz, 1H), 8.32 (s, 1H), 8.23 (d, J=8.1 Hz, 1H), 7.90-7.81 (m, 1H), 7.73 (dd, J=13.5, 2.1 Hz, 1H), 7.38 (t, J=9.0 Hz, 1H), 7.25-7.17 (m, 1H), 6.65 (d, J=5.4 Hz, 1H), 6.55 (d, J=2.7 Hz, 1H), 4.10-3.90 (m, 1H), 3.62-3.25 (m, 16H), 3.21 (s, 3H), 2.95-2.80 (m, 2H), 2.60-2.45 (m, 1H), 2.15-1.95 (m, 2H), 2.03 (s, 3H), 1.85-1.45 (m, 4H), 0.71-0.60 (m, 2H), 0.46-0.40 (m, 2H).

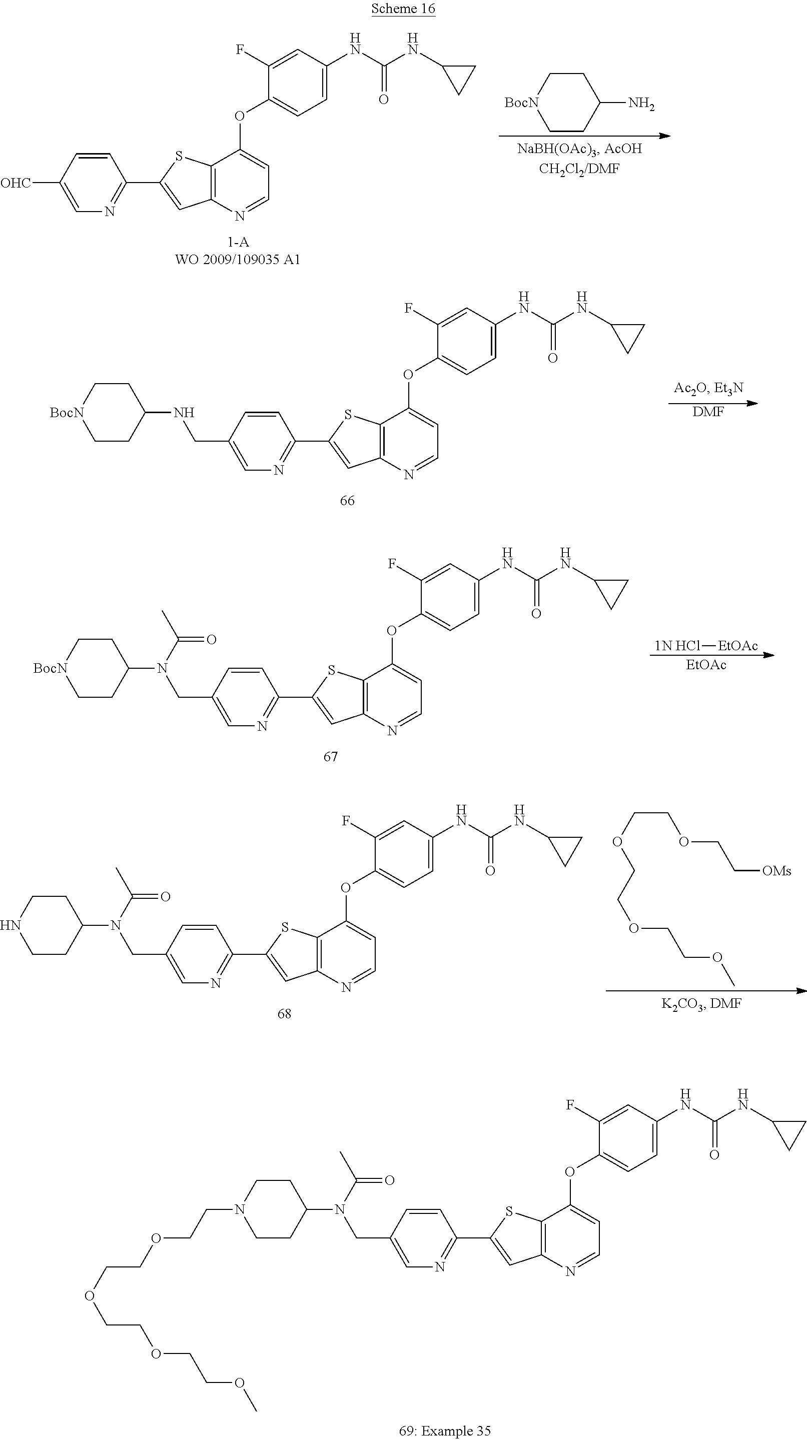

### Example 35

### N-(1-(2,5,8,11-Tetraoxamidecan-13-yl)piperidin-4-yl)-N-((6-(7-(4-(3-cyclopropylureido)-2-fluorophenoxy)thieno[3,2-b]pyridin-2-yl)pyridin-3-yl)methyl)acetamide (69)

### Step 1. tert-butyl 4-((6-(7-(4-(3-cyclopropylureido)-2-fluorophenoxy)thieno[3,2-b]pyridin-2-yl)pyridin-3-yl)methylamino)piperidine-1-carboxylate (66)

To a suspension of aldehyde 1-A (0.3 g, 0.669 mmol, WO 2009/109035 A1) in a mixture of DCM (9 mL) and DMF (3 mL) were added 4-amino-Boc-piperidine (0.27 g, 1.34 mmol) and acetic acid (80 μL, 1.34 mmol) at RT. The reaction mixture was stirred for 1.5 hr, treated with NaBH(OAc)₃ (0.425 g, 2.00 mmol) and stirred overnight, then quenched by addition of saturated NaHCO₃ solution and extracted with DCM/MeOH. The extract was washed with brine, dried over MgSO₄ and concentrated. The residue was purified by flash chromatography on silica gel (EtOAc/MeOH:93/7 to 84/16) to afford title compound 66 (0.355 g, 84% yield) as a pale brown. solid. ¹H NMR (300 MHz, DMSO-d₆) δ (ppm): 8.69 (s, 1H), 8.58 (brs, 1H), 8.51 (d, J=5.7 Hz, 1H), 8.30 (s, 1H), 8.22 (d, J=8.1 Hz, 1H), 7.91 (dd, J=8.4, 1.8 Hz, 1H), 7.72 (dd, J=13.5, 1.8 Hz, 1H), 7.37 (t, J=9.0 Hz, 1H), 7.25-7.15 (m, 1H), 6.64 (d, J=5.4 Hz, 1H), 6.55 (d, J=2.7 Hz, 1H), 3.85-3.75 (m, 2H), 3.81 (s, 2H), 2.90-2.65 (m, 2H), 1.85-1.75 (m, 2H), 1.45-1.35 (m, 1H), 1.39 (s, 9H), 1.25-1.10 (m, 2H), 0.71-0.62 (m, 2H), 0.48-0.40 (m, 2H).

### Step 2. tert-butyl 4-(N-((6-(7-(4-(3-cyclopropylureido)-2-fluorophenoxy)thieno[3,2-b]pyridin-2-yl)pyridin-3-yl)methyl)acetamido)piperidine-1-carboxylate (67)

To a solution of 66 (0.355 g, 0.561 mmol) in DMF (5 mL) were added TEA (0.2 mL, 1.4 mmol) and Ac₂O (0.12 mL, 1.12 mmol) at RT. The reaction mixture was stirred at 55° C. overnight, diluted with water and extracted with EtOAc/MeOH. The organic layer was washed with brine, dried over MgSO₄ and concentrated. The residue was purified by flash chromatography on silica gel (EtOAc/MeOH:90/10) to afford title compound 67 (0.314 g, 83%) as a white amorphous solid. ¹H NMR (300 MHz, DMSO-d₆) δ (ppm): 8.70 (s, 1H), 8.55-8.47 (m, 2H), 8.34 (s, 0.32H), 8.30 (s, 0.78H), 8.26 (d, J=8.4 Hz, 0.3H), 8.18 (d, J=8.4 Hz, 0.7H), 7.83-7.69 (m, 2H), 7.38 (t, J=9.0 Hz, 1H), 7.25-7.15 (m, 1H), 6.68-6.62 (m, 1H), 6.56 (d, J=2.7 Hz, 1H), 4.67 (s, 0.7H), 4.54 (s, 1.3H), 4.05-3.85 (m, 3H), 2.85-2.65 (m, 2H), 2.61-2.50 (m, 1H), 2.23 (s, 2H), 2.01 (s, 1H), 1.70-1.40 (m, 4H), 1.40-1.32 (m, 9H), 0.71-0.62 (m, 2H), 0.48-0.40 (m, 2H).

### Step 3. N-((6-(7-(4-(3-Cyclopropylureido)-2-fluorophenoxy)thieno[3,2-b]pyridin-2-yl)pyridin-3-yl)methyl)-N-(piperidin-4-yl)acetamide (68)

To a suspension of 67 (0.314 g, 0.465 mmol) in EtOAc (6 mL) was added 1N HCl-EtOAc (2.0 ml, 2.0 mmol) at RT. The reaction mixture was stirred overnight then concentrated and co-evaporated with EtOAc. The residue was purified by flash chromatography using Hi-Flash column (Yamazen Corporation) packed with amino silica gel (DCM/MeOH: 96/4 to 80/20) to afford title compound 68 (0.193 g, 72% yield) as a white amorphous solid. ¹H NMR (300 MHz, DMSO-d₆) δ (ppm): 8.71 (s, 1H), 8.54-8.46 (m, 2H), 8.34 (s, 0.3H), 8.30 (s, 0.7H), 8.26 (d, J=8.1 Hz, 0.3H), 8.18 (d, J=8.1 Hz, 0.7H), 7.82-7.69 (m, 2H), 7.38 (t, J=9.0 Hz, 1H), 7.25-7.15 (m, 1H), 6.68-6.62 (m, 1H), 6.57 (d, J=3.0 Hz, 1H), 4.66 (s, 0.7H), 4.54 (s, 1.3H), 3.90-3.75 (m, 1H), 3.00-2.90 (m, 2H), 2.61-2.40 (m, 3H), 2.20 (s, 2H), 1.99 (s, 1H), 1.65-1.40 (m, 4H), 0.71-0.62 (m, 2H), 0.48-0.40 (m, 2H).

### Step 4. N-(1-(2,5,8,11-Tetraoxamidecan-13-yl)piperidin-4-yl)-N-((6-(7-(4-(3-cyclopropylureido)-2-fluorophenoxy)thieno[3,2-b]pyridin-2-yl)pyridin-3-yl)methyl)acetamide (69)

To a solution of 68 (0.140 g, 0.244 mmol) in DMF (3 mL) were added K₂CO₃ (0.04 g, 0.293 mmol) and 2,5,8,11-tetraoxamidecan-13-yl methanesulfonate (105 mg, 0.366 mmol, K. Fukase, et. al. SynLett., 2005, 2342-2346) at room temperature. The reaction mixture was stirred at 60° C. overnight, diluted with water and extracted with EtOAc/MeOH. The organic layer was washed with brine, dried over MgSO₄ and concentrated. The residue was purified by flash chromatography on silica gel (DCM/MeOH) to afford title compound 69 (0.058 g, 31% yield) as a yellow solid film. ¹H NMR (300 MHz, DMSO-d₆) δ (ppm): 8.70 (s, 1H), 8.55-8.45 (m, 2H), 8.33 (s, 0.7H), 8.30 (s, 0.7H), 8.25 (d, J=8.1 Hz, 0.3H), 8.18 (d, J=8.1 Hz, 0.7H), 7.80-7.68 (m, 2H), 7.38 (t, J=9.0 Hz, 1H), 7.25-7.18 (m, 1H), 6.68-6.61 (m, 1H), 6.56 (d, J=2.7 Hz, 1H), 4.67 (s, 0.7H), 4.55 (s, 1.3H), 3.80-3.35 (m, 17H), 3.32 (s. 3H), 2.95-2.82 (m, 2H), 2.61-2.40 (m, 1H), 2.20 (s, 2H), 2.20-1.80 (m, 2H), 2.00 (s, 1H), 1.85-1.40 (m, 3H), 0.70-0.61 (m, 2H), 0.48-0.40 (m, 2H).

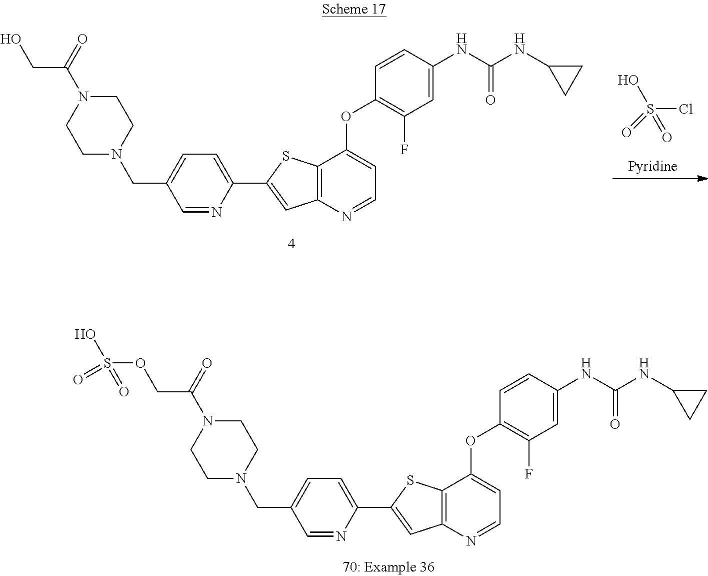 [SMILES: O=C(Nc1ccc(Oc2ccnc3cc(-c4ccc(CN5CCN(C(=O)CO)CC5)cn4)sc23)c(F)c1)NC1CC1 ; O=S(=O)(O)Cl ; O=C(Nc1ccc(Oc2ccnc3cc(-c4ccc(CN5CCN(C(=O)COS(=O)(=O)O)CC5)cn4)sc23)c(F)c1)NC1CC1]

### Example 36

### 2-(4-((6-(7-(4-(3-Cyclopropylureido)-2-fluorophenoxy)thieno[3,2-b]pyridin-2-yl)pyridin-3-yl)methyl)piperazin-1-yl)-2-oxoethyl hydrogen sulfate (70)

To a solution of 4 (0.20 g, 0.34 mmol) in pyridine (4.0 mL) was added dropwise chlorosulfuric acid (0.22 mL, 3.3 mmol) over 5 min under argon atmosphere, and the resultant mixture was stirred at 50° C. for 30 min. After cooling to room temperature, the mixture was added dropwise to the water (12 mL) then neutralized to pH 7.0 by addition of 1N NaOH aqueous solution. The precipitate was collected by filtration, was washed with water, dried in vacuo at room temperature to afford the title compound 70 (0.165 g, 72% yield) as a beige solid. ¹H NMR (300 MHz, DMSO-d₆) δ (ppm): 8.75 (brs, 1H), 8.73 (s, 1H), 8.56 (d, J=5.4 Hz, 1H), 8.45 (s, 1H), 8.41 (d, J=7.8 Hz, 1H), 8.10 (d, J=8.1 Hz, 1H), 8.74 (dd, J=13.5, 2.7 Hz, 1H), 7.39 (t, J=9.0 Hz, 1), 7.21 (d, J=9.3 Hz, 1H), 6.71 (d, J=5.4 Hz, 1H), 6.58 (brs, 1H), 4.70-4.10 (m, 6H), 3.30-2.90 (m, 4H), 2.60-2.51 (m, 1H), 0.70-0.62 (m, 2H), 0.46-0.40 (m, 2H). MS (m/z): 657.3 (M+H)⁺.

### Pharmaceutical Compositions

In some embodiments, the invention provides pharmaceutical compositions comprising a compound according to the invention and a pharmaceutically acceptable carrier, excipient, or diluent. Compositions of the invention may be formulated by any method well known in the art and may be prepared for administration by any route, including, without limitation, parenteral, oral, sublingual, transdermal, topical, intranasal, intratracheal, or intrarectal. In some embodiments, compositions of the invention are administered intravenously in a hospital setting. In some embodiments, administration may be by the oral route.

The characteristics of the carrier, excipient or diluent will depend on the route of administration. As used herein, the term “pharmaceutically acceptable” means a non-toxic material that is compatible with a biological system such as a cell, cell culture, tissue, or organism, and that does not interfere with the effectiveness of the biological activity of the active ingredient(s). Thus, compositions according to the invention may contain, in addition to the inhibitor, diluents, fillers, salts, buffers, stabilizers, solubilizers, and other materials well known in the art. The preparation of pharmaceutically acceptable formulations is described in, e.g., Remington's Pharmaceutical Sciences, 18th Edition, ed. A. Gennaro, Mack Publishing Co., Easton, Pa., 1990.

The active compound is included in the pharmaceutically acceptable carrier, excipient or diluent in an amount sufficient to deliver to a patient a therapeutically effective amount without causing serious toxic effects in the patient treated. The effective dosage range of a pharmaceutically acceptable derivative can be calculated based on the weight of the parent compound to be delivered. If the derivative exhibits activity in itself, the effective dosage can be estimated as above using the weight of the derivative, or by other means known to those skilled in the art.

### Inhibition of VEGF Receptor Signaling

In some embodiments the invention provides a method of inhibiting VEGF receptor signaling in a cell, comprising contacting a cell in which inhibition of VEGF receptor signaling is desired with an inhibitor of VEGF receptor signaling according to the invention. Because compounds of the invention inhibit VEGF receptor signaling, they are useful research tools for in vitro study of the role of VEGF receptor signaling in biological processes.

In some embodiments, inhibiting VEGF receptor signaling causes an inhibition of cell proliferation of the contacted cells.

### Assay Examples

### Inhibition of VEGF Activity

The following protocol was used to assay the compounds of the invention.

### Assay Example 1

### In Vitro Receptor Tyrosine Kinase Assay (VEGF Receptor KDR)

This test measures the ability of compounds to inhibit the enzymatic activity of recombinant human VEGF receptor enzymatic activity.

A 1.6-kb cDNA corresponding to the catalytic domain of VEGFR2 (KDR) (Genbank accession number AF035121 amino acid 806 to 1356) is cloned into the Pst I site of the pDEST20 Gateway vector (Invitrogen) for the production of a GST-tagged version of that enzyme. This construct is used to generate recombinant baculovirus using the Bac-to-Bac™ system according to the manufacturer's instructions (Invitrogen).

The GST-VEGFR2806-1356 protein is expressed in Sf9 cells (Spodoptera frugiperda) upon infection with recombinant baculovirus construct. Briefly, Sf9 cells grown in suspension and maintained in serum-free medium (Sf900 II supplemented with gentamycin) at a cell density of about 2×106 cells/ml are infected with the above-mentioned viruses at a multiplicity of infection (MOI) of 0.1 during 72 hours at 27° C. with agitation at 120 rpm on a rotary shaker. Infected cells are harvested by centrifugation at 398 g for 15 min. Cell pellets are frozen at −80° C. until purification is performed.

All steps described in cell extraction and purification are performed at 4° C. Frozen Sf9 cell pellets infected with the GST-VEGFR2806-1356 recombinant baculovirus are thawed and gently resuspended in Buffer A (PBS pH 7.3 supplemented with 1 μg/ml pepstatin, 2 μg/ml Aprotinin and leupeptin, 50 μg/ml PMSF, 50 μg/ml TLCK and 10 μM E64 and 0.5 mM DTT) using 3 ml of buffer per gram of cells. Suspension is Dounce homogenized and 1% Triton X-100 is added to the homogenate after which it is centrifuged at 22500 g, 30 min., 4° C. The supernatant (cell extract) is used as starting material for purification of GST-VEGFR2806-1356.

The supernatant is loaded onto a GST-agarose column (Sigma) equilibrated with PBS pH 7.3. Following a four column volume (CV) wash with PBS pH 7.3+1% Triton X-100 and 4 CV wash with buffer B (50 mM Tris pH 8.0, 20% glycerol and 100 mM NaCl), bound proteins are step eluted with 5 CV of buffer B supplemented with 5 mM DTT and 15 mM glutathion. GST-VEGFR2806-1356 enriched fractions from this chromatography step are pooled based on U.V. trace i.e. fractions with high O.D.280. Final GST-VEGFR2806-1356 protein preparations concentrations are about 0.7 mg/ml with purity approximating 70%. Purified GST-VEGFR2806-1356 protein stocks are aliquoted and frozen at −80° C. prior to use in enzymatic assay.

Inhibition of VEGFR/KDR is measured in a DELFIA™ assay (Perkin Elmer). The substrate poly(Glu4,Tyr) is immobilized onto black high-binding polystyrene 96-well plates. The coated plates are washed and stored at 4° C. During the assay, the enzyme is pre-incubated with inhibitor and Mg-ATP on ice in polypropylene 96-well plates for 4 minutes, and then transferred to the coated plates. The subsequent kinase reaction takes place at 30° C. for 10-30 minutes. ATP concentrations in the assay are 0.6 uM for VEGFR/KDR (2× the Km). Enzyme concentration is 5 nM. After incubation, the kinase reactions are quenched with EDTA and the plates are washed. Phosphorylated product is detected by incubation with Europium-labeled anti-phosphotyrosine MoAb. After washing the plates, bound MoAb is detected by time-resolved fluorescence in a Gemini SpectraMax reader (Molecular Devices). Compounds are evaluated over a range of concentrations, and IC₅₀ values (concentration of compounds giving 50% inhibition of enzymatic activity) are determined. The results are shown in Table 8.

TABLE 8

| | VEGFR | P_ERK |
|---|---|---|
| Cmpd No | IC50UM | IC50UM |
| 6 | 0.009 | 0.0004 |
| 30 | 0.018 | 0.07 |
| 7 | 0.006 | 0.0002 |
| 33 | 0.107 | |
| 38 | 0.024 | |
| 40 | 0.024 | |
| 39 | 0.033 | 0.006 |
| 60 | 0.01 | 0.0015 |
| 69 | 0.012 | 0.025 |
| 18 | 0.006 | |
| 19 | 0.02 | |
| 45 | 0.003 | |
| 13 | 0.01 | |
| 12 | 0.013 | |
| 22 | 0.013 | |
| 23 | 0.018 | |
| 65 | 0.007 | 0.002 |
| 49 | 0.008 | |
| 46 | 0.011 | |
| 8 | 0.013 | |
| 51 | 0.013 | |
| 50 | 0.008 | 0.0008 |
| 52 | 0.013 | |
| 55 | 0.002 | |
| 54 | 0.002 | |
| 56 | 0.002 | |
| 25 | 0.053 | |
| 24 | 0.015 | |
| 26 | 0.013 | |
| 47 | 0.012 | |
| 48 | 0.013 | |

### Assay Example 2

### In Vivo Choroidal Neovascularization (CNV) Model

This test measures the capacity of compounds to inhibit CNV progression. CNV is the main cause of severe vision loss in patients suffering from age-related macular degeneration (AMD).

Male Brown-Norway rats (Charles River Japan Co., Ltd.) were used in these studies.

Rats were anesthetized by intraperitoneal injection of pentobarbital, and the right pupil was dilated with 0.5% tropicamide and 0.5% phenylephrine hydrochloride. The right eye received 6 laser burns between retinal vessels using a slit lamp delivery system of Green laser Photocoagulator (Nidex Inc., Japan), and microscope slide glass with 10 mg/mL hyaluronic acid (SIGMA) used as a contact lens. The laser power was 200 mW for 0.1 second and spot diameter was 100 μm. At the time of laser burn, bubble production was observed; which is an indication of rupture of Bruch's membrane which is important for CNV generation.

After animals were anesthetized, and the right pupil dilated (as mentioned above), the right eye of the animal received the compound or vehicle by an injection (3 μL/eye) at doses of 3 or 10 nmol/eye on Day 3. The compounds were dissolved or suspended in CBS, PBS, or other adequate vehicles before injection.

On Day 10, the animals were anesthetized with ether, and high molecular weight fluorescein isothiocyanate (FITC)-dextran (SIGMA, 2×10⁶ MW) was injected via a tail vein (20 mg/rat). About 30 min after FITC-dextran injection, animals were euthanized by ether or carbon dioxide, and the eyes were removed and fixed with 10% formaline neutral buffer solution. After over 1 hour of fixation, RPE-choroid-sclera flat mounts were obtained by removing cornea, lens and retina from the eyeballs. The flat mounts were mounted in 50% glycerol on a microscope slide, and the portion burned by laser was photographed using a fluorescence microscope (Nikon Corporation, excitation filter: 465-495 nm, absorption filter: 515-555 nm). The CNV area was obtained by measurement of hyper-fluorescence area observed on the photograph using Scion image.

The average CNV area of 6 burns was used as an individual value of CNV area, and the average CNV area of compound treated group was compared with that of the vehicle-treated group. Results with some compounds of the present invention are shown in Table 9.

TABLE 9

| | Dose | Inhibition of |
|---|---|---|
| Cmpd No. | (nmol/eye) | CNV Progression (%) |
| 6 | 3 | 20.9 |
| 30 | 3 | 26.4 |
| 7 | 3 | 18.1 |
| 39 | 3 | 24.7 |
| 65 | 3 | 32.1 |
| 49 | 3 | 20.9 |
| 46 | 3 | 24.8 |
| 51 | 10 | 64.3 |
| 50 | 10 | 80.6 |
| 18 | 3 | 42.9 |
| 13 | 3 | 41.6 |
| 12 | 3 | 47 |
| 23 | 3 | 44.2 |
| 25 | 3 | 20.9 |
| 24 | 3 | 28.9 |

### Assay Example 3

### VEGF-Dependent Erk Phosphorylation

Cells and Growth Factor:

HUVEC cells are purchased from Cambrex Bio Science Walkersville, Inc and cultured according to the vendor's instructions. The full-length coding sequence of VEGF₁₆₅ is cloned using the Gateway Cloning Technology (Invitrogen) for baculovirus expression Sf9 cells. VEGF₁₆₅ is purified from conditioned media using a NaCl gradient elution from a HiTrap heparin column (GE Healthcare Life Sciences) followed by an imidazole gradient elution from a HiTrap chelating column (GE Healthcare Life Sciences), then buffer stored in PBS supplemented with 0.1% BSA and filter sterilized

Cell Assays:

Cells are seeded at 8000 cells/well of a 96 wells plate and grown for 48 hours. Cells are then grown overnight in serum and growth factor-free medium and exposed for 1.5 h to compounds dilutions. Following a 15 min incubation in medium, VEGF₁₆₅ (150 ng/ml) cells are lysed in ice-cold lysis buffer (50 mM HEPES, pH 7.4, 150 mM NaCl, 1.5 mM MgCl₂, 1% Triton X-100, 10% glycerol) containing 1 mM 4-(2 aminoethyl)benzenesulfonyl fluoride hydrochloride, 200 μM sodium orthovanadate, 1 mM sodium fluoride, 10 μg/mL leupeptin, 10 μg/mL aprotinin, 1 μg/mL pepstatin and 50 μg/mL Na-p-tosyl-L-lysine chloromethyl ketone hydrochloride and processed as Western blots to detect anti-phospho ERK1/2 (T202/Y204) (Cell Signaling Technologies).

Western Blot Analysis:

lysates samples from single treatment wells are separated on 5-20% SDS-PAGE gels and immunobloting is performed using Immobilon polyvinylidene difluoride membranes (Amersham) according to the manufacturer's instructions. The blots are washed in Tris-buffered saline with 0.1% Tween 20 detergent (TBST) and probed for antibodies against phospho-Thr202/Tyr204-ERK (Cell signaling technologies. Chemiluminescence detection (Amersham, ECL plus) is performed according to the manufacturer's instructions using a Storm densitometer (GE Healthcare; 800 PMT, 100 nM resolution) for imaging and densitometry analysis. Values of over the range of dilution are used to prepare IC₅₀ curves using a 4-parameter fit model. These curves are calculated using GraFit

5.0 software.

### Assay Example 4

### In Vivo Solid Tumor Disease Model

This test measures the capacity of compounds to inhibit solid tumor growth.

Tumor xenografts are established in the flank of female athymic CD1 mice (Charles River Inc.), by subcutaneous injection of 1×106 U87, A431 or SKLMS cells/mouse. Once established, tumors are then serially passaged s.c. in nude mice hosts. Tumor fragments from these host animals are used in subsequent compound evaluation experiments. For compound evaluation experiments female nude mice weighing approximately 20 g are implanted s.c. by surgical implantation with tumor fragments of ˜30 mg from donor tumors. When the tumors are approximately 100 mm3 in size (˜7-10 days following implantation), the animals are randomized and separated into treatment and control groups. Each group contains 6-8 tumor-bearing mice, each of which is ear-tagged and followed individually throughout the experiment.

Mice are weighed and tumor measurements are taken by calipers three times weekly, starting on Day 1. These tumor measurements are converted to tumor volume by the well-known formula (L+W/4)3 4/3π. The experiment is terminated when the control tumors reach a size of approximately 1500 mm³. In this model, the change in mean tumor volume for a compound treated group/the change in mean tumor volume of the control group (non-treated or vehicle treated)×100 (ΔT/ΔC) is subtracted from 100 to give the percent tumor growth inhibition (% TGI) for each test compound. In addition to tumor volumes, body weight of animals is monitored twice weekly for up to 3 weeks

### Assay Example 5

### VEGF-Induced Retinal Vascular Permeability in Rabbits

This test measures the capacity of compounds to inhibit VEGF-induced retinal vascular permeability. Vascular permeability is the cause of severe vision loss in patients suffering from age-related macular degeneration (AMD). Female Dutch rabbits (˜2 kg; Kitayama LABES CO., LTD, Nagano, Japan) are anesthetized with pentobarbital and topically with 0.4% oxybuprocaine hydrochloride. Test articles or vehicle are injected into vitreous cavity after the dilation of the pupils with 0.5% tropicamide eye drop. Recombinant human VEGF₁₆₅ (500 ng; Sigma-Aldrich Co., St Louis, Mo.) is injected intravitreously 48 hr prior to the measurement of vitreous fluorescein concentration. Rabbits are anesthetized with pentobarbital and sequentially injected sodium fluorescein (2 mg/kg) via the ear vein. Pupils are dilated with 0.5% tropicamide eye drop, and ocular fluorescein levels are measured using the FM-2 Fluorotron Master (Ocumetrics, Mountain View, Calif.) 30 min after fluorescein injection. The fluorescein concentrations in vitreous are obtained at data points that are 0.25 mm apart from posterior-end along an optical axis. Vitreous fluorescence concentration is considered fluorescein leakage from retinal vasculature. The average fluorescence peaks of the test article treated groups are compared with that of the vehicle-treated group.

### Assay Example 6

### Stability Measurements Under Basic Conditions

To 0.28 mg of compound 50 (92.6% purity) was added 200 μL of 0.1 M NaOH in D-PBS(−) (Wako Pure Chemical Co., Ltd., pH 7.4) at room temperature. The suspension was kept for 24 hours at room temperature followed by the purity determination using UPLC method, upon dissolving the suspension in DMSO (800 μL) and injecting 0.5 μL of the resultant solution into Waters ACQUITY UPLC H class instrument. Column: ACQUITY UPLC BEH C18 1.7 um (2.1×100 mm). Conditions: 7 min run per sample using the following gradient eluting systems:

1) 0.1% formic acid in water/0.1% formic acid=95/5 for 0.2 min;
2) 0.1% formic acid in water/0.1% formic acid=95/5 to 5/95 (gradient) for 4.3 min;
3) 0.1% formic acid in water/0.1% formic acid=5/95 for 1.0 min;
4) 0.1% formic acid in water/0.1% formic acid=95/5 for 1.5 min.
Table 10 contains results for compound 50: its purity after the incubation at different pH and temperatures.

TABLE 10

| | | | | | | | .1M |
|---|---|---|---|---|---|---|---|
| | | | | | | | OH |
| | | | | | | | 0° C. |
| 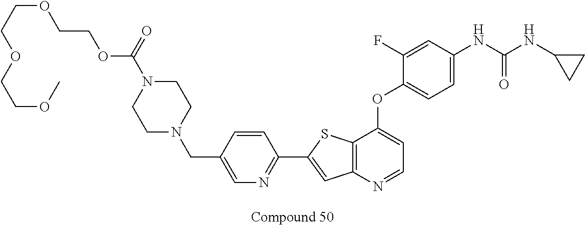 [SMILES: COCCOCCOCCOC(=O)N1CCN(Cc2ccc(-c3cc4nccc(Oc5ccc(NC(=O)NC6CC6)cc5F)c4s3)nc2)CC1] | 92.30 | 93.91 | 93.81 | 92.07 | 93.84 | 94.95 | 91.98 |

Table 10 demonstrates that compounds of the present invention show good stability and may withstand basic conditions during the formulation process.

1. A compound having the Formula (I): 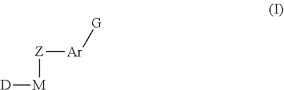
including N-oxides, hydrates, solvates, tautomers, pharmaceutically acceptable salts, prodrugs and complexes thereof, and racemic and scalemic mixtures, diastereomers and enantiomers thereof, wherein,
D is selected from the group consisting of an aromatic, heteroaromatic, cycloalkyl or heterocyclic ring system, C₁-C₆alkyl-heterocyclyl-C(O)—, C₁-C₆alkyl-heterocyclyl-C₁-C₆alkyl-N(R⁶)—C(O)—, (R⁶)(R⁶)N—C(O)—O-heterocyclyl-C(O)—, heterocyclyl-C(O)—, PivO-heterocyclyl-C(O)—, C₁-C₆alkyl-O—C(O)-heterocyclyl-C(O)—, C₁-C₆alkyl-C(O)—N(R⁶)-heterocyclyl-C(O)—, (C₁-C₆alkyl)(Box)N-heterocyclyl-C(O)—, HO-heterocyclyl-C(O)—, HO—C(O)-heterocyclyl-C(O)—, C₁-C₆alkyl-C(O)—O-heterocyclyl-C(O)—, (R⁶)(R⁶)N—C₁-C₆alky-N(R⁶)—C(O)-heterocyclyl-C(O)—, C₁-C₆alkyl-heterocyclyl-C(O)-heterocyclyl-C(O)— and (R⁶)(R⁶)N-heterocyclyl-C(O)—, wherein each of the aromatic, heteroaromatic, cycloalkyl and heterocyclic groups is optionally substituted with 1 or more independently selected R³⁸;
M is an optionally substituted fused heterocyclic moiety;
Z is selected from the group consisting of —O—, —S(O)₀₋₂— and —NR⁵—, wherein R⁵ is selected from the group consisting of H, optionally substituted C₁-C₅alkyl, an optionally substituted (C₁-C₅)acyl and C₁-C₆ alkyl-O—C(O), wherein C₁-C₆ alkyl is optionally substituted;
Ar is a group of the formula C, 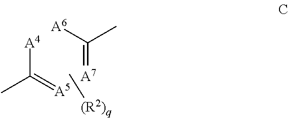
wherein,
A⁴, A⁵, A⁶ and A⁷ are independently selected from the group consisting of N and —CH—, with the proviso that no more than two of A⁴, A⁵, A⁶ and A⁷ can be N,
wherein Ar is optionally substituted with R²;
G is a group B-L-T, wherein
B is selected from the group consisting of a covalent bond, —N(R¹³)—, —N(SO₂R¹³)—, —O—, —S(O)₀₋₂ and —C(═O)—;
L is selected from the group consisting of a covalent bond, —C(═S)N(R¹³)—, —C(═NR¹⁴)N(R¹³)—, —SO₂N(R¹³)—, —SO₂—, —C(═O)N(R¹³)—, —N(R¹³)—, —C(═O)C₁₋₂alkyl-N(R¹³)—, —N(R¹³)C₁₋₂alkyl-C(═O)—, —C(═O)C₀₋₁alkyl-C(═O)N(R¹³)—, —C₀₋₄alkylene, —C(═O)C₀₋₁alkyl-C(═O)OR³—, —C(═NR¹⁴)—C₀₋₁alkyl-C(═O)—, —C(═O)—, —C(═O)C₀₋₁alkyl-C(═O)— and an optionally substituted four to six-membered heterocyclyl containing between one and three annular heteroatoms including at least one nitrogen, wherein the alkyl and alkylene are optionally substituted; and
T is selected from the group consisting of —H, —R¹³, —C₀₋₄alkyl, —C₀₋₄alkyl-Q, —O—C₀₋₄alkyl-Q, —C₀₋₄alkyl-O-Q, —N(R¹³)C₀₋₄alkyl-Q, —SO₂C₀₋₄alkyl-Q, —C(═O)C₀₋₄alkyl-Q, —C₀₋₄-alkyl-N(R¹³)Q and —C(═O)N(R¹³)—C₀₋₄alkyl-Q, wherein each C₀₋₄alkyl is optionally substituted;
wherein
R³⁸ is selected from the group consisting of R^{37b}O-C₁-C₆alkyl-C(O)-heterocyclyl-CH₂—, R³⁷O—(CH₂)ⱼ—[(CH₂)ᵢO]ₓ—(CH₂)ᵢ₁-heterocyclyl-CH₂—, heterocyclyl-CH₂—, R³⁷O—(CH₂)ⱼ—[(CH₂)ᵢO]ₓ—(CH₂)ⱼ—C(O)O—(CH₂)ᵢ₁—C(O)-heterocyclyl-CH₂—, R³⁷O—(CH₂)ⱼ—[(CH₂)ᵢO]ₓ—(CH₂)ᵢ₁—N(R²⁰¹)-heterocyclyl-CH₂—, R³⁷O—(CH₂)ⱼ—[(CH₂)ᵢO]ₓ—(CH₂)ᵢ₁—O-aryl-NHCH₂—, R³⁷O—(CH₂)ⱼ—[(CH₂)ᵢO]ₓ—(CH₂)ⱼ—C(O)O—(CH₂)ᵢ₁—C(O)-heterocyclyl-N(R²⁰¹)CH₂—, R³⁷O—(CH₂)ⱼ—[(CH₂)ᵢO]ₓ—(CH₂)ᵢ₁—C(O)-heterocyclyl-CH₂—, R³⁷O—(CH₂)ⱼ—[(CH₂)ᵢO]ₓ—(CH₂)ᵢ₁—OC(O)-heterocyclyl-CH₂—, R³⁷O—(CH₂)ⱼ-[(CH₂)ᵢO]ₓ—(CH₂)ᵢ₁—NHC(O)-heterocyclyl-CH₂—, R³⁷O—(CH₂)ⱼ—[(CH₂)ᵢO]ₓ-(CH₂)ᵢ₁—N(R²⁰⁰)C(O)-heterocyclyl-CH₂—, R³⁷O—(CH₂)ⱼ—[(CH₂)ᵢO]ₓ—(CH₂)ᵢ₁—O-heterocyclyl-CH₂—, R³⁷O—(CH₂)ⱼ—[(CH₂)ᵢO]ₓ—(CH₂)ⱼ—C(O)O—(CH₂)ᵢ₁-heterocyclyl-N(R²⁰¹)CH₂—;
each R⁶ is independently H or C₁-C₆alkyl;
R³⁷ is selected from the group consisting of H, C₁-C₆alkyl and C₃-C₁₀cycloalkyl;
R^{37b} is selected from the group consisting of (HO)₂P(═O)—, R²⁰¹HNCH(R²⁰⁰)C(O)NHCH(R²⁰⁰)C(O)—, R²⁰¹NHCH(R²⁰⁰)C(O)—, R²⁰¹CH(R²⁰⁰)CH(R²⁰⁰)C(O)—, R²⁰¹HNCH(R²⁰⁰)C(O)NHCH(R²⁰⁰)C(O)— and is HO—SO₂—;
j is an integer ranging from 0 to 4;
i is 2 or 3;
x is an integer ranging from 0 to 6;
i1 is 2 or 3;
R²⁰⁰ is selected from the group consisting of H, C₁-C₆-alkyl, C₃-C₆-cycloalkyl, aryl-(C₁-C₆-alkyl)-, OR²⁰¹, NHR²⁰¹ and SR²⁰¹; wherein the alkyl, aryl and cyclylalkyl moieties of the foregoing R²⁰⁰ groups are optionally substituted;
R²⁰¹ is selected from the group consisting of H, C₁-C₆-alkyl; aryl, aryl-(C₁-C₆-alkyl)-; (C₁-C₆-aryl-(C₁-C₆-alkyl)C(O); (C₁-C₆-alkyl)OC(O); aryl-(C₁-C₆-alkyl)C(O); (C₁-C₆-alkyl)NHC(O); (C₁-C₆-alkyl)NHC(O)O—; (C₁-C₆-alkyl)NHC(O)NH—; (C₁-C₆)SO₂—, wherein the alkyl, and aryl moieties of the foregoing R²⁰¹ groups are optionally substituted;
R² at each occurrence is independently selected from the group consisting of —H, halogen, trihalomethyl, —CN, —NO₂, —NH₂, —OR³, —NR³R⁴, —S(O)₀₋₂R³, —S(O)₂NR³R³, —C(O)OR³, —C(O)NR³R³, —N(R³)SO₂R³, —N(R³)C(O)R³, —N(R³)CO₂R³, —C(O)R³, C₁-C₄ alkoxy, C₁-C₄ alkylthio, —O(CH₂)-aryl, —O(CH₂)-heteroaryl, —(CH₂)₀₋₅(aryl), —(CH₂)₀₋₅(heteroaryl), C₁-C₆ alkyl, C₂-C₆ alkenyl, C₂-C₆ alkynyl, —CH₂(CH₂)₀₋₄-T², wherein T² is selected from the group consisting of —OH, —OMe, —OEt, —NH₂, —NHMe, —NMe₂, —NHEt and —NEt₂, and wherein the aryl, heteroaryl, C₁-C₆ alkyl, C₂-C₆ alkenyl, and C₂-C₆ alkynyl are optionally substituted; and
q is an integer from 0 to 4;
n is an integer ranging from 0 to 4;
each R³ is independently selected from the group consisting of —H and R⁴;
R⁴ is selected from the group consisting of a (C₁-C₆)alkyl, an aryl, a lower arylalkyl, a heterocyclyl and a lower heterocyclyl-alkyl, each of which is optionally substituted, or
R³ and R⁴, taken together with a common nitrogen to which they are attached, form an optionally substituted five- to seven-membered heterocyclyl, the optionally substituted five- to seven-membered heterocyclyl optionally containing at least one additional annular heteroatom selected from the group consisting of N, O, S and P
R¹³ is selected from the group consisting of —H, —CN, —NO₂, —NH₂, —OR³, —NR³R⁴, —S(O)₀₋₂R³, —S(O)₂NR³R³, —C(O)OR³, —C(O)NR³R³, —N(R³)SO₂R³, —N(R³)C(O)R³, —N(R³)CO₂R³, —C(O)R³, —C(O)SR³, C₁-C₄ alkoxy, C₁-C₄ alkylthio, —O(CH₂)ₙ₅aryl, —O(CH₂)ₙ₅heteroaryl, —(CH₂)ₙ₅(aryl), —(CH₂)ₙ₅(heteroaryl), C₁-C₆ alkyl, C₂-C₆ alkenyl, C₂-C₆ alkynyl, —CH₂(CH₂)₀₋₄-T², an optionally substituted C₁₋₄ alkylcarbonyl, and a saturated or unsaturated three- to seven-membered carboxyclic or heterocyclic group, wherein T² is selected from the group consisting of —OH, —OMe, —OEt, —NH₂, —NHMe, —NMe₂, —NHEt and —NEt₂, and wherein the aryl, heteroaryl, C₁-C₆ alkyl, C₂-C₆ alkenyl, and C₂-C₆ alkynyl are optionally substituted;
two R¹³, together with the atom or atoms to which they are attached, can combine to form a heteroalicyclic optionally substituted with between one and four of R⁶⁰, wherein the heteroalicyclic can have up to four annular heteroatoms, and the heteroalicyclic can have an aryl or heteroaryl fused thereto, in which case the aryl or heteroaryl is optionally substituted with an additional one to four of R⁶⁰;
n5 is an integer ranging from 0 to 6
R⁶⁰ is selected from the group consisting of —H, halogen, trihalomethyl, —CN, —NO₂, —NH₂, —OR³, —NR³R⁴, —S(O)₀₋₂R³, —SO₂NR³R³, CO₂R³, —C(O)NR³R³, —N(R³)SO₂R³, —N(R³)C(O)R³, —N(R³)CO₂R³, —C(O)R³, an optionally substituted (C₁-C₆)alkyl, an optionally substituted aryl, an optionally substituted heteroarylalkyl and an optionally substituted arylalkyl;
two R⁶⁰, when attached to a non-aromatic carbon, can be oxo;
R¹⁴ is selected from the group —H, —NO₂, —NH₂, —N(R³)R⁴, —CN, —OR³, an optionally substituted (C₁-C₆)alkyl, an optionally substituted heteroalicyclyl-alkyl, an optionally substituted aryl, an optionally substituted arylalkyl and an optionally substituted heteroalicyclic,
Q is a three- to ten-membered ring system, optionally substituted with zero, one or more of R²⁰;
R²⁰ is selected from the group consisting of —H, halogen, trihalomethyl, —CN, —NO₂, —NH₂, —OR³, —OCF₃, —NR³R⁴, —S(O)₀₋₂R³, —S(O)₂NR³R³, —C(O)OR³, —C(O)NR³R³, —N(R³)SO₂R³, —N(R³)C(O)R³, —N(R³)C(O)OR³, —C(O)R³, —C(O)SR³, C₁-C₄ alkoxy, C₁-C₄ alkylthio, —O(CH₂)ₙ₆aryl, —O(CH₂)ₙ₆heteroaryl, —(CH₂)ₙ₆(aryl), —(CH₂)ₙ₆(heteroaryl), C₁-C₆ alkyl, C₂-C₆ alkenyl, C₂-C₆ alkynyl, —CH₂(CH₂)₀₋₄-T², an optionally substituted C₁₋₄ alkylcarbonyl, C₁₋₄ alkoxy, an amino optionally substituted by C₁₋₄ alkyl optionally substituted by C₁₋₄ alkoxy, —(CH₂)ₙ₆P(═O)(C₁-C₆alkyl)₂, a saturated or unsaturated three- to seven-membered carboxyclic or heterocyclic group, —SiMe₃ and —SbF₅; and
n6 is an integer ranging from 0 to 6.

2. The compound according to claim 1, wherein the compound is

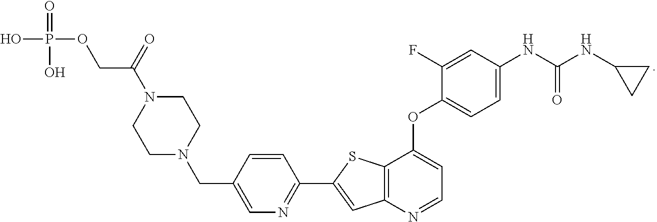 [SMILES: O=C(Nc1ccc(Oc2ccnc3cc(-c4ccc(CN5CCN(C(=O)COP(=O)(O)O)CC5)cn4)sc23)c(F)c1)NC1CC1]

3. The compound according to claim 1, wherein the compound is

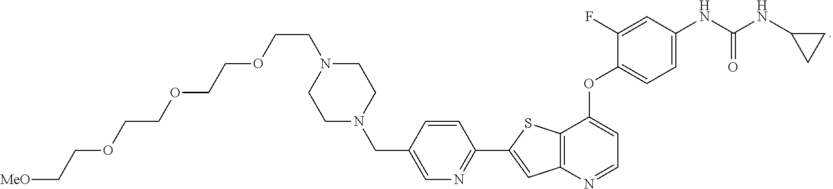 [SMILES: COCCOCCOCCOCCN1CCN(Cc2ccc(-c3cc4nccc(Oc5ccc(NC(=O)NC6CC6)cc5F)c4s3)nc2)CC1]

4. The compound according to claim 1, wherein the compound is

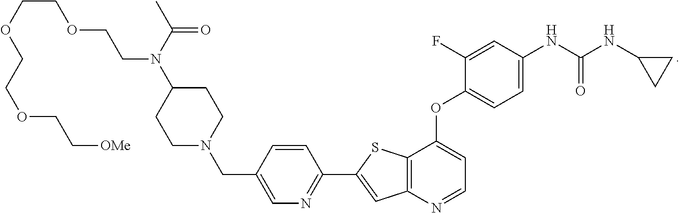 [SMILES: COCCOCCOCCOCCN(C(C)=O)C1CCN(Cc2ccc(-c3cc4nccc(Oc5ccc(NC(=O)NC6CC6)cc5F)c4s3)nc2)CC1]

5. The compound according to claim 1, wherein the compound is

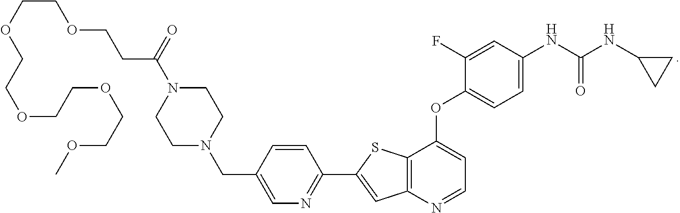 [SMILES: COCCOCCOCCOCCOCCC(=O)N1CCN(Cc2ccc(-c3cc4nccc(Oc5ccc(NC(=O)NC6CC6)cc5F)c4s3)nc2)CC1]

6. The compound according to claim 1, wherein the compound is

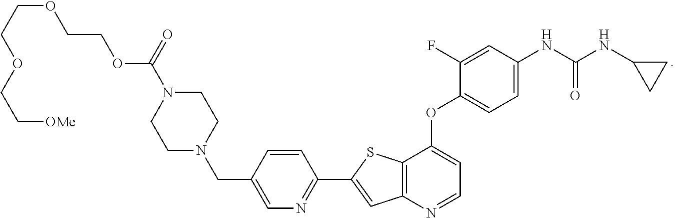 [SMILES: COCCOCCOCCOC(=O)N1CCN(Cc2ccc(-c3cc4nccc(Oc5ccc(NC(=O)NC6CC6)cc5F)c4s3)nc2)CC1]

7. The compound according to claim 1, wherein the compound is

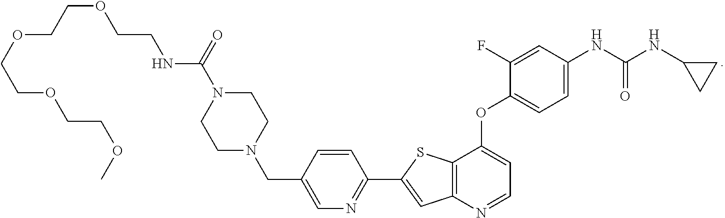 [SMILES: COCCOCCOCCOCCNC(=O)N1CCN(Cc2ccc(-c3cc4nccc(Oc5ccc(NC(=O)NC6CC6)cc5F)c4s3)nc2)CC1]

8. The compound according to claim 1, wherein the compound is

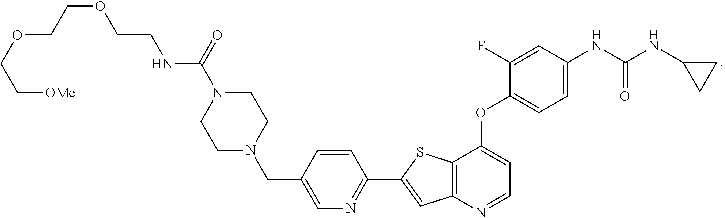 [SMILES: COCCOCCOCCNC(=O)N1CCN(Cc2ccc(-c3cc4nccc(Oc5ccc(NC(=O)NC6CC6)cc5F)c4s3)nc2)CC1]

9. The compound according to claim 1, wherein the compound is

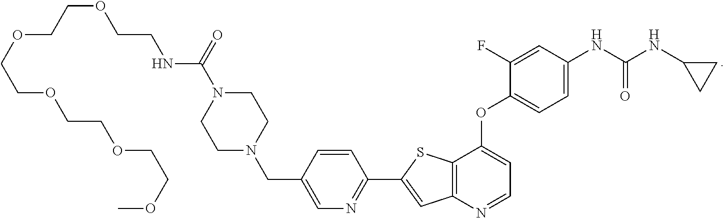 [SMILES: COCCOCCOCCOCCOCCNC(=O)N1CCN(Cc2ccc(-c3cc4nccc(Oc5ccc(NC(=O)NC6CC6)cc5F)c4s3)nc2)CC1]

10. The compound according to claim 1, wherein the compound is

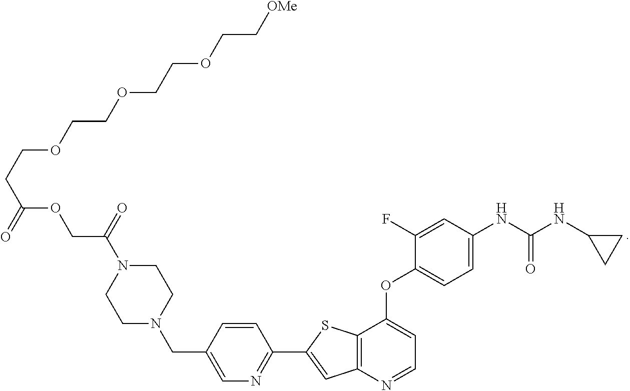 [SMILES: COCCOCCOCCOCCC(=O)OCC(=O)N1CCN(Cc2ccc(-c3cc4nccc(Oc5ccc(NC(=O)NC6CC6)cc5F)c4s3)nc2)CC1]

11. The compound according to claim 1, wherein the compound is

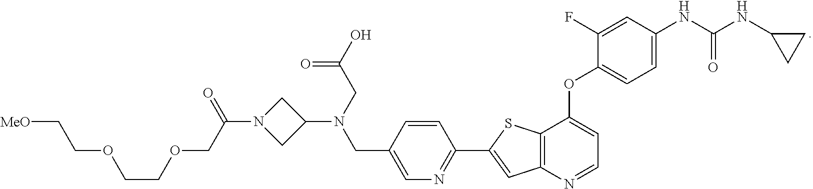 [SMILES: COCCOCCOCC(=O)N1CC(N(CC(=O)O)Cc2ccc(-c3cc4nccc(Oc5ccc(NC(=O)NC6CC6)cc5F)c4s3)nc2)C1]

12. A composition comprising a compound according to claim 1 and a pharmaceutically acceptable carrier.

13. A method of treating an ophthalmic disease, condition or disorder, the method comprising administering to a patient in need thereof a therapeutically effective amount of a compound according to claim 1 or a composition thereof, wherein the ophthalmic disease, disorder or condition is selected from the group consisting of (a) a disease, disorder or condition caused by choroidal angiogenesis, (b) diabetic retinopathy and (c) retinal oedema.

14. The method according to claim 13, wherein the ophthalmic disease, disorder or condition is age-related macular degeneration.
